(19) United States
(12) Patent Application Publication
(10) Pub. No.: US 2005/0107386 A1
(43) Pub. Date: May 19, 2005

(54) Methods of treating diseases and disorders by targeting multiple kinases

(76) Inventors: Rama Krishna Narla, San Diego, CA (US); Steven Sakata, San Diego, CA (US)

Correspondence Address: JONES DAY, 222 EAST 41ST ST, NEW YORK, NY, 10017, US

(21) Appl. No.: 10/994,093

(22) Filed: Nov. 19, 2004

Related U.S. Application Data
(60) Provisional application No. 60/523,859, filed on Nov. 19, 2003.
(60) Provisional application No. 60/608,929, filed on Nov. 19, 2003.

(51) Int. Cl. A61K 31/519; A61K 31/53; A61K 31/4709; A61K 31/4196

(52) U.S. Cl. 514/243; 514/262.1; 514/314; 514/383; 514/249; 514/248; 514/310

(57) ABSTRACT

The present invention is directed to the use of single agents, which are compounds that target two or more kinases simultaneously, thus substantially avoiding resistance to therapy. The invention provides methods for the use for, administration to, and treatment of individuals having a variety of diseases or conditions associated with the activity of two or more kinases, comprising administration of one or more single agents, either alone or in combination with other therapies for the same disease or condition.

This application claims benefit of U.S. Provisional Application Ser. No. 60/523,859, filed Nov. 19, 2003, and claims benefit of U.S. Provisional Application Ser. No. 60/608,929, filed Nov. 19, 2003, each which is incorporated by reference herein in its entirety

### 1. FIELD OF THE INVENTION

The present invention encompasses methods for simultaneously modulating the activities of multiple kinases or kinase pathways, including those implicated in processes important for cell survival, proliferation, growth and malignant transformation, motility and invasion. As such, the invention also encompasses methods for treating, preventing, or managing conditions, diseases, and disorders associated with protein kinases or protein kinase pathways, such as proliferative disorders and cancer, inflammatory disorders, including diabetes and obesity and abnormal angiogenesis and diseases related thereto. In particular, the methods contemplated by the invention comprise treating, preventing, or managing a disease, condition, or disorder with single-agent therapies that specifically target multiple kinases or kinase pathways. In one embodiment, the single agent therapy preferably modulates the activity of multiple kinases or kinase pathways over that of other kinases; in other words, the effect of the single agent therapy is selective for specific sets of kinases. In sum, the invention contemplates the identification and use of single agents that target the right combination of multiple pathways that are clinically effective in a particular disease setting.

### 2. BACKGROUND OF THE INVENTION

The connection between abnormal protein phosphorylation and the cause or consequence of diseases has been known for over 20 years. Accordingly, protein kinases have become a very important group of drug targets. See Cohen, Nature, 1:309-315 (2002). Various protein kinase inhibitors have been used clinically in the treatment of a wide variety of diseases, such as cancer and chronic inflammatory diseases, including diabetes and stroke. See Cohen, Eur. J. Biochem., 268:5001-5010 (2001).

The protein kinases are a large and diverse family of enzymes that catalyze protein phosphorylation and play a critical role in cellular signaling. Protein kinases may exert positive or negative regulatory effects, depending upon their target protein. Protein kinases are involved in specific signaling pathways which regulate cell functions such as, but not limited to, metabolism, cell cycle progression, cell adhesion, vascular function, apoptosis, and angiogenesis. Malfunctions of cellular signaling have been associated with many diseases, the most characterized of which include cancer and diabetes. The regulation of signal transduction by cytokines and the association of signal molecules with protooncogenes and tumor suppressor genes have been well documented. Similarly, the connection between diabetes and related conditions, and deregulated levels of protein kinases, has been demonstrated. See e.g., Sridhar et al. Pharmaceutical Research, 17(11):1345-1353 (2000). Viral infections and the conditions related thereto have also been associated with the regulation of protein kinases. Park et al. Cell 101 (7), 777-787 (2000).

Protein kinases can be divided into broad groups based upon the identity of the amino acid(s) that they target (serine/threonine, tyrosine, lysine, and histidine). For example, tyrosine kinases include receptor tyrosine kinases (RTKs), such as growth factors and non-receptor tyrosine kinases, such as the src kinase family. There are also dual-specific protein kinases that target both tyrosine and serine/threonine, such as cyclin dependent kinases (CDKs) and mitogen-activated protein kinases (MAPKs). Any particular cell contains many protein kinases, some of which phosphorylate other protein kinases. Some protein kinases phosphorylate many different proteins, others phosphorylate only a single protein. Not surprisingly, there are numerous classes of protein kinases. Upon receiving a signal, some proteins may also undergo auto-phosphorylation.

The protein tyrosine kinases (PTKs) compose a large family of kinases that regulate cell to cell signals involved in growth, differentiation, adhesion, motility, and death. Robinson et al., Oncogene 19:5548-5557 (2000). Members of the tyrosine kinase include, but are not limited to, Yes, BMX, Syk, EphA1, FGFR3, RYK, MUSK, JAK1 and EGFR. Tyrosine kinases are distinguished into two classes, i.e., the receptor type and non-receptor type tyrosine kinases. Interestingly, the entire of family of tyrosine kinases is quite large—consisting of at least 90 characterized kinases with at least 58 receptor type and at least 32 nonreceptor type kinases comprising at least 30 total subfamilies. Robinson et al., Oncogene 19:5548-5557 (2000). Tyrosine kinases have been implicated in a number of diseases in humans, including diabetes and cancer. Robinson et al. at page 5548. Tyrosine kinases are often involved in most forms of human malignancies and have been linked to a wide variety of congenital syndromes. Robertson et al., Trends Genet. 16:265-271 (2000).

The non-receptor tyrosine kinases represent a group of intracellular enzymes that lack extracellular and transmembrane sequences. Currently, over 32 families of non-receptor tyrosine kinases have been identified. Robinson et al., Oncogene 19:5548-5557 (2000). Examples are Src, Btk, Csk, ZAP70, Kak families. In particular, the Src family of non-receptor tyrosine kinase family is the largest, consisting of Src, Yes, Fyn, Lyn, Lck, Blk, Hck, Fgr and Yrk protein tyrosine kinases. The Src family of kinases have been linked to oncogenesis, cell proliferation and tumor progression. A detailed discussion of non-receptor protein tyrosine kinases is available in Oncogene 8:2025-2031 (1993). Many of these protein tyrosine kinases have been found to be involved in cellular signaling pathways involved in various pathological conditions including but not limited to cancer and hyperproliferative disorders and immune disorders.

The cyclin dependent kinases CDKs represent a group of intracellular enzymes that control progression through the cell cycle and have essential roles in cell proliferation. See Cohen, Nature, 1:309-315 (2002). Examples of CDKs include, but are not limited to, cyclin dependent kinase 2 (CDK2), cyclin dependent kinase 7 (CDK7), cyclin dependent kinase 6 (CDK6) and cell division control 2 protein (CDC2). CDKs have been implicated in the regulation of transitions between different phases of the cell cycle, such as the progression from a quiescent stage in GI (the gap between mitosis and the onset of DNA replication for a new round of cell division) to S (the period of active DNA synthesis), or the progression from G₂ to M phase, in which active mitosis and cell division occur. See e.g., the articles compiled in Science, vol. 274 (1996), pp. 1643-1677; and Ann. Rev. Cell Dev Biol., vol. 13 (1997), pp. 261-291. CDK complexes are formed through association of a regulatory cyclin subunit (e.g., cyclin A, B1, B2, D1, D2, D3, and E) and a catalytic kinase subunit (e.g., cdc2 (CDK1), CDK2, CDK4, CDK5, and CDK6). As the name implies, CDKs display an absolute dependence on the cyclin subunit in order to phosphorylate their target substrates, and different kinase/cyclin pairs function to regulate progression through specific portions of the cell cycle. CDKs have been implicated in various disease states, including but not limited to, those displaying the cancer phenotype, various neoplastic disorders and in neurological disorders. Hunter, Cell 100: 113-127 (2000).

The mitogen activated protein (MAP) kinases participate in the transduction of signals to the nucleus of the cell in response to extracellular stimuli. Examples of MAP kinases include, but are not limited to, mitogen activated protein kinase 3 (MAPK3), mitogen-activated protein kinase 1 (ERK2), mitogen-activated protein kinase 7 (MAPK7), mitogen-activated protein kinase 8 (JNK1), mitogen-activated protein kinase 14 (p38 alpha), mitogen-activated protein kinase 10 (MAPK 10), JNK3 alpha protein kinase, stress-activated protein kinase JNK2 and mitogen-activated protein kinase 14 (MAPK14). MAP kinases are a family of proline-directed serine/threonine kinases that mediate signal transduction from extracellular receptors or heath shock, or UV radiation. See Sridhar et al., Pharmaceutical Research, 17:11 1345-1353 (2000). MAP kinases activate through the phosphorylation of theonine and tyrosine by dual-specificity protein kinases, including tyrosine kinases such as growth factors. Cell proliferation and differentiation have been shown to be under the regulatory control of multiple MAP kinase cascades. See Sridhar et al., Pharmaceutical Research, 17:11 1345-1353 (2000). As such, the MAP kinase pathway plays critical roles in a number of disease states. For example, defects in activities of MAP kinases have been shown to lead to aberrant cell proliferation and carcinogenesis. See Hu et al., Cell Growth Differ. 11:191-200 (2000); and Das et al., Breast Cancer Res. Treat. 40:141 (1996). Moreover, MAP kinase activity has also been implicated in insulin resistance associated with type-2 diabetes. See Virkamaki et al., J. Clin. Invest. 103:931-943 (1999).

The p90 ribosomal S6 kinases (Rsk) are serine/threonine kinases. The Rsk family members function in mitogen-activated cell growth and proliferation, differentiation, and cell survival. Examples of members of the Rsk family of kinases include, but are not limited to, ribosomal protein S6 kinase, 90 kDa, polypeptide 2 (Rsk3), ribosomal protein S6 kinase, 90 kDa, polypeptide 6 (Rsk4), ribosomal protein S6 kinase, 90 kDa, polypeptide 3 (Rsk2) and ribosomal protein S6 kinase, 90 kDa, polypeptide 1 (Rsk1/p90Rsk). The Rsk family members are activated by extracellular signal-related kinases ½ and phosphoinositide-dependent protein kinase 1. Frodin and Gammeltoft, Mol. Cell. Endocrinol. 151:65-77 (1999). Under basal conditions, RSK kinases are localized in the cytoplasm of cells and upon stimulation by mitogens, the activated (phosphorylated by extracellular-related kinase) RSK transiently translocates to the plasma membrane where they become fully activated. The fully activated RSK phosphorylates substrates that are involved in cell growth, proliferation, differentiation, and cell survival. Richards et al., Curr. Biol. 9:810-820 (1999); Richards et al., Mol. Cell. Biol. 21:7470-7480 (2001). RSK signaling pathways have also been associated with the modulation of the cell cycle. Gross et al., J. Biol. Chem. 276(49): 46099-46103 (2001). Current data suggests that small molecules that inhibit Rsk may be useful therapeutic agents for the prevention and treatment of cancer and inflammatory diseases.

Members of the checkpoint protein kinase family are serine/threonine kinases that play an important role in cell cycle progression. Examples of members of the checkpoint family include, but are not limited to, CHK1 and CHK2. Checkpoints are control systems that coordinate cell cycle progression by influencing the formation, activation and subsequent inactivation of the cyclin-dependent kinases. Checkpoints prevent cell cycle progression at inappropriate times, maintain the metabolic balance of cells while the cell is arrested, and in some instances can induce apoptosis (programmed cell death) when the requirements of the checkpoint have not been met. See e.g., O'Connor, Cancer Surveys, 29: 151-182 (1997); Nurse, Cell, 91: 865-867 (1997); Hartwell et al., Science, 266: 1821-1828 (1994); Hartwell et al., Science, 246: 629-634 (1989). Members of the checkpoint family of kinases have been implicated in cell proliferative disorders, cancer phenotypes and other diseases related to DNA damage and repair. Kohn, Mol. Biol. Cell 10:2703-2734 (1999); Ohi and Gould, Curr. Opin. Cell Biol. 11:267-273 (1999); Peng, et al., Science 277:1501-1505 (1997).

Aurora kinases are a family of multigene mitotic serine-threonine kinases that functions as a class of novel oncogenes. These kinases comprise aurora-A and aurora-B members. Aurora kinases are hyperactivated and/or over-expressed in several solid tumors including but not limited to, breast, ovary, prostate, pancreas, and colorectal cancers. In particular aurora-A is a centrosome kinase that plays an important role cell cycle progression and cell proliferation. Aurora-A is located in the 20q13 chromosome region that is frequently amplified in several different types of malignant tumors such as colorectal, breast and bladder cancers. There is also a high correlation between aurora-A and high histo-prognostic grade aneuploidy, making the kinase a potential prognostic vehicle. Inhibition of aurora kinase activity could help to reduce cell proliferation, tumor growth and potentially tumorigenesis. A detailed description of aurora kinase function is reviewed in Oncogene 21:6175-6183 (2002).

The Rho-associated coiled-coil-containing protein serine/threonine kinases ROCK-I and ROCK-II are thought to play a major role in cytoskeletal dynamics by serving as downstream effectors of the Rho/Rac family of cytokine- and growth factor-activated small GTPases. ROCKs phosphorylate various substrates, including, but not limited to, myosin light chain phosphatase, myosin light chain, ezrin-radixin-moesin proteins and LIM (for Lin11, Isl1 and Mec3) kinases. ROCKs also mediate the formation of actin stress fibers and focal adhesions in various cell types. ROCKs have an important role in cell migration by enhancing cell contractility. They are required for tail retraction of monocytes and cancer cells, and a ROCK inhibitor has been used to reduce tumor-cell dissemination in vivo. Recent experiments have defined new functions of ROCKs in cells, including centrosome positioning and cell-size regulation, which might contribute to various physiological and pathological states. See Nature Reviews Mol. Cell Biol. 4, 446-456 (2003). The ROCK family members are attractive intervention targets for a variety of pathologies, including cancer and cardiovascular disease. For example, Rho kinase inhibitors can be useful therapeutic agents for hypertension, angina pectoris, and asthma. Furthermore, Rho is expected to play a role in peripheral circulation disorders, arteriosclerosis, inflammation, and autoimmune disease and as such, is a useful target for therapy.

The 70 kDa ribosomal S6 kinase (p70S6K) is activated by numerous mitogens, growth factors and hormones. Activation of p70S6K occurs through phosphorylation at a number of sites and the primary target of the activated kinase is the 40S ribosomal protein S6, a major component of the machinery involved in protein synthesis in mammalian cells. In addition to its involvement in regulating translation, p7OS6K activation has been implicated in cell cycle control, neuronal cell differentiation, regulation of cell motility and a cellular response that is important in tumor metastases, immunity and tissue repair. Modulation of p70S6 kinase activity may have therapeutic implications in disorders such as cancer, inflammation, and various neuropathies. A detailed discussion of p70S6K kinases can be found in Prog. Cell Cycle Res. 1:21-32 (1995), and Immunol Cell Biol. 78(4):447-51 (2000).

Glycogen synthase kinase 3 (GSK-3) is a ubiquitously expressed constitutively active serine/threonine kinase that phosphorylates cellular substrates and thereby regulates a wide variety of cellular functions, including development, metabolism, gene transcription, protein translation, cytoskeletal organization, cell cycle regulation, and apoptosis. GSK-3 was initially described as a key enzyme involved in glycogen metabolism, but is now known to regulate a diverse array of cell functions. Two forms of the enzyme, GSK-3α and GSK-3β, have been previously identified. The activity of GSK-3β is negatively regulated by protein kinase B/Akt and by the Wnt signaling pathway. Small molecules inhibitors of GSK-3 may, therefore, have several therapeutic uses, including the treatment of neurodegenerative diseases, diabetes type II, bipolar disorders, stroke, cancer, and chronic inflammatory disease. Reviewed in Role of glycogen synthase kinase-3 in cancer: regulation by Wnts and other signaling pathways (Adv Cancer Res.;84:203-29, 2002); Glycogen synthase kinase 3 (GSK-3) inhibitors as new promising drugs for diabetes, neurodegeneration, cancer, and inflammation (Med Res Rev.; 22(4):373-84, 2002); Role of glycogen synthase kinase-3 in the phosphatidylinositol 3-Kinase/Akt cell survival pathway. (J. Biol Chem., 273(32): 19929-32, 1998).

Because protein kinases regulate nearly every cellular process, including metabolism, cell proliferation, cell differentiation, and cell survival, they are attractive targets for therapeutic intervention for various disease states. For example, cell-cycle control and angiogenesis, in which protein kinases play a pivotal role are cellular processes associated with numerous disease conditions such as but not limited to cancer, inflammatory diseases, abnormal angiogenesis and diseases related thereto, atherosclerosis, macular degeneration, diabetes, obesity, and pain.

Protein kinases have become attractive targets for the treatment of cancers. Fabbro et al., Pharmacology & Therapeutics 93:79-98 (2002). It has been proposed that the involvement of protein kinases in the development of human malignancies may occur by: (1) genomic rearrangements (e.g., BCR-ABL in chronic myelogenous leukemia), (2) mutations leading to constitutively active kinase activity, such as acute myelogenous leukemia and gastrointestinal tumors, (3) deregulation of kinase activity by activation of oncogenes or loss of tumor suppressor functions, such as in cancers with oncogenic RAS, (4) deregulation of kinase activity by over-expression, as in the case of EGFR and (5) ectopic expression of growth factors that can contribute to the development and maintenance of the neoplastic phenotype. Fabbro et al., Pharmacology & Therapeutics 93:79-98 (2002).

Certain cancers are associated with angiogenesis. Angiogenesis is the growth of new capillary blood vessels from pre-existing vasculature. Risau, W., Nature 386:671-674 (1997). It has been shown that protein kinases can contribute to the development and maintenance of the neoplastic phenotype. Fabbro et al., Pharmacology & Therapeutics 93:79-98 (2002). For example, VEGF A-D and their four receptors have been implicated in phenotypes that involve neovascualrization and enhanced vascular permeability, such as tumor angiogenesis and lymphangiogenesis. Matter, A., Drug Discov. Today 6:1005-1023 (2001).

Cardiovascular disease (“CVD”) accounts for nearly one quarter of total annual deaths worldwide. Vascular disorders such as atherosclerosis and restenosis result from dysregulated growth of the vessel walls and the restriction of blood flow to vital organs. Various kinase pathways, e.g. JNK, are activated by atherogenic stimuli and regulated through local cytokine and growth factor production in vascular cells. Yang et al., Immunity 9:575 (1998). Ischemia and ischemia coupled with reperfusion in the heart, kidney or brain result in cell death and scar formation, which can ultimately lead to congestive heart failure, renal failure or cerebral dysfumction. In organ transplantation, reperfusion of previously ischemic donor organs results in acute leukocyte-mediated tissue injury and delay of graft function. Ischemia and reperfusion pathways are mediated by various kinases. For example, the JNK pathway has been linked to leukocyte-mediated tissue damage. Li et al., Mol. Cell. Biol. 16:5947-5954 (1996). Finally, enhanced apoptosis in cardiac tissues has also been linked to kinase activity. Pombo et al., J. Biol. Chem. 269:26546-26551 (1994).

The elucidation of the intricacy of protein kinase pathways and the complexity of the relationship and interaction among and between the various protein kinases and kinase pathways highlights the importance of developing pharmaceutical agents capable of acting as protein kinase modulators, regulators or inhibitors that have beneficial activity on multiple kinases or multiple kinase pathways.

It has been recognized that a single agent approach that specifically targets one kinase or one kinase pathway may be inadequate to treat diseases and disorders, in particular cancer, for several reasons. The single agent approach is a limited approach for the treatment of very complex diseases, conditions and disorders. For example, mathematical models have suggested that 5 to 7 mutations are necessary for the progression from a normal cell to malignant transformation. Additionally, other events (such as DNA methylation) can occur that modify the expression of existing genes.

Thus, it is widely recognized that cancer is the result of alterations in multiple pathways, in particular protein kinase pathways that are associated with processes such as cell growth, proliferation, apoptosis, motility, or invasion. In a majority of cancers, a common feature is the simultaneous overexpression and/or hyper-activation of a variety of protein kinases, such as receptor and non-receptor kinases, serine/threonine kinases, PI3 kinases and cell cycle associated kinases. In fact, several of these kinases, either alone or in conjunction with other kinases, have been implicated in a number of processes important for cell survival, proliferation, growth and malignant transformation, motility and invasion leading to metastasis and angiogenesis or inflammation, and diseases, disorders, and conditions associated therewith.

Accordingly, blocking one target kinase may not be clinically sufficient because there are multiple target kinases that affect the progression of a condition, disease, or disorder. In addition, blocking one target kinase may not be clinically sufficient because redundant kinase-mediated pathways and alternative oncogenic or inflammatory mechanisms may compensate for the blocked target kinase. Moreover, the use of a single agent can also increase the chances that resistance to that agent will develop.

It has therefore been suggested that due to the complexity of intracellular signaling cascades of protein kinase pathways, agents that affect multiple pathways simultaneously may be required for meaningful clinical activity. Although it has been suggested that a single agent that provides combinatorial effects is an attractive notion, there is a need to identify and use single agents that target the right combination of multiple pathways that are clinically effective in a particular disease setting. Indeed, it is known that some kinase drugs, such as Gleevec®, do target several kinases at once. Gleevec® primarily targets a mutant fusion protein containing the abl kinase, which is created by a 9:22 chromosomal translocation event; Gleevec® also targets c-kit, a tyrosine kinase implicated in gastrointestinal stromal tumors (GIST). However, in recent clinical trials, patients have developed resistance to Gleevec® or have shown incomplete response to treatment.

51 It has also been suggested that a single agent could target the primary cause of a disease and thereby regulate the downstream pathways merely by targeting one specific molecule. For example, a viral infection often affects the regulation of multiple kinases and it has been found that an agent that inhibits viral replication can, as a result, inhibit the activation of all of the multiple kinases activated by the virus. However, due to the complexity of intracellular signaling cascades of protein kinase pathways and the diseases that they are involved in, there is a need for single agents that affect multiple pathways simultaneously by directly targeting the specific kinases and/or kinase pathways. In particular, there is a need to identify single agents that are capable of simultaneously targeting multiple kinases or kinase pathways and capable of directly and selectively targeting a particular kinase and/or kinase pathway while not affecting other kinases and/or pathways. For example, rather than using a single agent that inhibits the activation of all of the multiple kinases activated by a virus in a viral infection, the single agent can simultaneously and selectively inhibit certain kinases that are activated.

An additional strategy for targeting multiple kinases or kinase pathways has been to use cocktails of single target drugs. Although the use of drug combinations in the clinic has shown promise, there is still a need to develop single agent drugs that are capable of targeting multiple kinases or kinase pathways because the use of a single agent is more pragmatic for a variety of reasons, including the ease and simplicity of administration. Moreover, it is already known that certain treatment regimens utilizing multiple agents which each target single pathways can be associated with complications, including unpleasant side effects and toxicity.

Accordingly, there remains a need for the development of methods comprising the use of a single agent drug capable of targeting specific sets of kinases or kinase pathways, in particular the right combination of multiple targets thereby achieving clinical efficacy.

### 3. BRIEF SUMMARY OF THE INVENTION

The present invention is based in part on the discovery that small molecule kinase inhibitors capable of simultaneously inhibiting multiple kinases (also referred herein as mixed kinases) have more potent anti-proliferative activity than certain specific kinase inhibitors. Because inhibition of one specific kinase or a specific pathway is not always sufficient to elicit significant clinical response and might lead to rapid resistance, the instant invention encompasses methods comprising the use of a single agent that is capable of targeting more than one kinase or kinase pathway. The methods of the invention therefore comprise methods for affecting processes important for cell survival, proliferation, growth and malignant transformation, motility and invasion leading to angiogenesis and metastasis by simultaneously targeting multiple protein kinases or protein kinase pathways.

The present invention therefore is directed to the use of agents able to target certain kinases, that is, to regulate or to modulate the activity, function or level of certain kinases and/or the level of expression of genes encoding certain kinases, or regulate or modulate certain kinase-mediated signaling pathways; i.e., able to target the kinase or kinase-mediated pathway. Such agents are referred to as “single agents” or “mixed kinase agents.”

Applicants have discerned that CDK kinases, Rsk kinases, checkpoint kinases, MAPK kinases, Src kinases, and the kinases Fes, Lyn, Syk are important to, in particular, the progression of proliferative disorders. In one embodiment, therefore, the agent targets two or more of the following: kinases from the src kinase family, kinases from the Rsk kinase family, kinases from the CDK family, kinases from the MAPK kinase family, and tyrosine kinases such as Fes, Lyn, and Syk kinases. The agent may target two or more kinases of the same family, or may target kinases representing two or more kinase families or classes.

In preferred embodiments, the invention provides a method of treating a disease, disorder or condition comprising targeting multiple kinases or kinase pathways that have been implicated in the disease, disorder, or condition.

The instant invention also encompasses the use of these single agents in combination with one or more agents that specifically target a single kinase or kinase pathway. In other embodiments, the single agent may be used with other therapies such as conventional forms of chemotherapy, antiangiogenics, nucleoside analogs, proteosome inhibitors, and the like; radiation therapy; cytokine therapy; surgery; or any of the other therapies disclosed in Section 5.4.3, below.

As such, the methods of the invention are also useful as an adjunct to existing and/or experimental therapies.

According to the methods of the present invention, identification of modulators that can simultaneously target, regulate or inhibit multiple kinases or kinase pathways critically implicated in various diseases or disorders can be achieved by using the assays disclosed in Examples 4-83, or by using in vivo models described in Example 3.

### 3.1 Definitions

As used herein, “resistance” and “resistant” refer to target kinases that show detectably reduced response to a particular kinase modulator over time. For example, a resistant kinase is one that shows detectably more activity when exposed at a time after the first exposure to a single agent of the invention, as compared to the response of the kinase upon the first exposure of the kinase to the single agent. Also by way of example, a kinase may be resistant, or show a detectable lessening of response, to a single inhibitor, or a mixture of inhibitors. The resistance of the kinase to a particular modulator may arise from a mutation, change in post-translational modification, upregulation or downregulation of the gene encoding the kinase, increased or decreased clearance of the kinase from tissues, or any other cause. Resistance may also develop as a result of the upregulation of alternate kinase-mediated pathways the function of which is at least partly redundant to the targeted kinase's pathway.

As used herein, the terms “simultaneous” and “simultaneously” mean over the duration of a particular administration and effect of a single agent, where a single agent is administered to an individual, and refer to the effect the therapy has on all targeted kinases, whether or not those effects are demonstrable at the same particular point in time during the course of administration and effect. A particular single agent, combination of single agents, or combination of one or more single agents and one or more other compounds target two or more kinases simultaneously over a course of therapy. For example, where a single agent is administered to an individual and an effect on one targeted kinase is detectable immediately (e.g., within the first few minutes after administration), and an effect on a second targeted kinase is detectable only later, the single agent is said to affect the two kinases simultaneously. “Simultaneous” and “simultaneously” also refer to similar effects in vitro resulting from contact with a single agent.

As used herein, “side effect” indicates an effect of a particular single agent, combination of single agents, or combination of single agent(s) with other treatments other than the immediate effect of modulating the activity of (e.g., inhibiting) the two or more kinases targeted.

As used herein, a single agent acts “directly” or “specifically” on two or more target kinases by interacting with each of the kinases to modify, inhibit or regulate the kinases activities, for example, inhibiting the kinase in a competitive, noncompetitive or uncompetitive manner; altering the level of the kinase in a tissue by interacting with transcriptional complexes specific for the kinase; altering the post-translational modification of those specific kinases, and the like. For example, whether a compound directly targets two or more kinases can be determined using the in vitro assays as described herein, or may be determined using other art-known kinase assays. A single agent does not act “directly” on a target kinase where the effect on the target kinase is solely the result of the single agent's effect on a kinase that modifies the target kinase.

As used herein, a “therapeutically effective amount” refers to that amount of the therapeutic agent sufficient to result in a detectable amelioration of one or more symptoms of a disorder. In certain embodiments, a “therapeutically effective amount” refers to that amount of the therapeutic agent sufficient to destroy, modify, control or remove primary, regional or metastatic cancer tissue. As such, therapeutically effective amount also refers to the amount of therapeutic agent sufficient to delay or minimize the spread of cancer. In another example, “therapeutically effective amount” refers to the amount effective to detectably reduce inflammation, or the production of cytokines or proliferation of cells associated with inflammation. A therapeutically effective amount may also refer to the amount of the therapeutic agent that provides a therapeutic benefit in the treatment or management of a disease, condition, or disorder, or to reduce the incidence of recurrence or onset of one or more symptoms of a disease, condition or disorder in a population of individuals.

As used herein, a “prophylactically effective amount” refers to that amount of the prophylactic agent sufficient to result in the prevention of the recurrence or onset of one or more symptoms of a disease, condition, or disorder.

In certain embodiments, a “prophylactically effective amount” refers to that amount of the prophylactic agent sufficient to result in the prevention of the recurrence or spread of cancer. As such, the term also refers to the amount of prophylactic agent sufficient to prevent the recurrence or spread of cancer or the occurrence of cancer in an individual, including but not limited to those individuals predisposed to cancer or previously exposed to carcinogens. A prophylactically effective amount may also refer to the amount of the prophylactic agent that provides a prophylactic benefit in the prevention of a disease, condition, or disorder.

As used herein, the terms “single agent” and “single agents” refer to any therapeutic or prophylactic agent(s) that can be used in the prevention, treatment, management or amelioration of one or more symptoms of a disease, condition, or disorder, wherein the single agent simultaneously targets more than one kinase or kinase pathway. A single agent is preferably not a macromolecule (e.g., protein, polypeptide, polysaccharide, polynucleotide) and is preferably a small organic molecule having a molecular weight of less than 1000 daltons. A single agent may be a peptide or a polynucleotide fragment (e.g., an aptamer). Preferably, the single agent is orally bioavailable and/or bioactive, and is bioactive and bioavailable when delivered, e.g., intramuscularly, intravenously, or by inhalation. The term “single agent” does not include any naturally-occurring protein in its native form.

The terms “agent” or “therapeutic agent” or “prophylactic agent” as used herein refers to any molecule, e.g., protein, polypeptide, peptide, antibody, antibody fragment, oliogonucleotides, antisense oliogonucleotides, large molecule, or small molecule (less than 10 kD) that targets a kinase, that is, modulates, regulates, enhances, blocks, inhibits, reduces or neutralizes the function, activity or expression of a protein kinase.

As used herein, the phrase “non-responsive/refractory” is used to describe patients treated with currently available therapies, wherein the therapy is not clinically adequate to relieve symptoms of the patients such that these patients need additional effective therapy, e.g., remain unsusceptible to therapy. The phrase can also describe patients who respond to therapy yet suffer from side effects, relapse, develop resistance, etc. In connection with anti-cancer therapy, in certain embodiments, “non-responsive/refractory” means that at least some significant portion of the cancer cells are not killed or their cell division arrested. The determination of whether the cancer cells are “non-responsive/refractory” can be made either in vivo or in vitro by any method known in the art for assaying the effectiveness of treatment on cancer cells, using the art-accepted meanings of “refractory” in such a context.

In various embodiments, a cancer is “non-responsive/refractory” where the number of cancer cells has not been significantly reduced, or has increased.

As used herein, the phrase “low tolerance” refers to a state in which the patient suffers from side effects from treatment to the extent that the patient does not benefit from and/or will not continue therapy because of the adverse effects.

As used herein, the terms “individual,” “subject” and “patient” are used interchangeably. As used herein, a subject is preferably a mammal such as a non-primate (e.g., cows, pigs, horses, cats, dogs, rats etc.) or a primate (e.g., monkey and human).

As used herein, the terms “manage,” “managing” and “management” refer to the beneficial effects that a subject derives from a prophylactic or therapeutic agent, which does not result in a cure of the disease. In certain embodiments, a subject is administered one or more prophylactic or therapeutic agents to “manage” a disease so as to prevent the progression or worsening of the disease.

As used herein, the terms “prevent,” “preventing” and “prevention” refer to the prevention of the recurrence or onset of one or more symptoms of a disease, disorder or condition. In one embodiment, the terms refer to prevention of the recurrence or onset of one or more symptoms of an autoimmune or inflammatory disease in a subject resulting from the administration of a prophylactic or therapeutic agent. In another embodiment, the terms prevent,” “preventing” and “prevention” also refer to the prevention of the recurrence, spread or onset of cancer in a subject resulting from the administration of a prophylactic or therapeutic agent.

As used herein, the terms “treat,” “treatment” and “treating” refer to the amelioration of one or more symptoms associated with a disease, disorder or condition that results from the administration of one or more prophylactic or therapeutic agents. In certain embodiments, such terms refer to a reduction in proliferative activity of a cell resulting from the administration of one or more prophylactic or therapeutic agents to a subject in need thereof. In other embodiments, the terms refer to a reduction in inflammatory activity of a cell resulting from the administration of one or more prophylactic or therapeutic agents to a subject in need thereof. In certain other embodiments, such terms refer to the reduction of abnormal angiogenesis activity of a cell resulting from the administration of one or more prophylactic or therapeutic agents to a subject in need thereof.

In connection with anti-cancer therapy, as used herein, the terms “treat,” “treating” and “treatment” refer to the eradication, removal, modification, reduction of the spread, or reduction of the rate of spread, or control of primary, regional, or metastatic cancer tissue that results from the administration of one or more prophylactic or therapeutic agents. In certain embodiments, such terms refer to the minimizing or delay of the spread of cancer resulting from the administration of one or more prophylactic or therapeutic agents to a subject with such a disease.

### 4. BRIEF DESCRIPTION OF THE FIGURES

FIG. 1 depicts results of the administration of CC001 on tumor size in an SCID mouse xenograft model in which the mice received HCT-116 cells.

FIG. 2 depicts results of the administration of CC00 2 or CC003 on tumor size in an SCID mouse xenograft model in which the mice received HCT-116 cells.

FIG. 3 depicts results of the administration of CC001, with or without Camptosar, on tumor size in an SCID mouse xenograft model in which the mice received HCT-116 cells.

### 5. DETAILED DESCRIPTION OF THE INVENTION

This invention encompasses methods for treating, preventing, or managing conditions, diseases, or disorders involving more than one protein kinase or protein kinase pathway. The present invention is based in part on the discovery that small molecule kinase inhibitors capable of simultaneously inhibiting multiple kinases (also referred herein as mixed kinases) have more potent anti-proliferative activity than certain specific kinase inhibitors. The present inventors have discovered methods for affecting processes important for cell survival, proliferation, growth and malignant transformation, motility and invasion leading to angiogenesis and metastasis by simultaneously modulating protein kinase pathways involving two or more of the following: kinases from the src kinase family, kinases from the Rsk kinase family, kinases from the CDK family, kinases from the MAPK kinase family, and tyrosine kinases such as Fes, Lyn, and Syk kinases. Specifically, the inventors have discovered single agents capable of simultaneously targeting multiple kinases and/or kinase pathways.

Because inhibition of one specific kinase or a specific pathway is not always sufficient to elicit significant clinical response and might lead to rapid resistance, the instant invention encompasses methods comprising the use of a single agent that is capable of targeting more than one kinase or kinase pathway. The methods of the invention are capable of circumventing the challenges faced by a single agent that targets a single kinase or kinase pathway.

The methods of the invention comprise methods for affecting processes important for cell survival, proliferation, growth and malignant transformation, motility and invasion leading to angiogenesis and metastasis by simultaneously targeting multiple protein kinases or protein kinase pathways. The methods comprise using a single agent that modulates, regulates or inhibits more than one kinase or kinase pathway.

As such, the methods of the invention comprise methods comprising targeting multiple kinases or kinase pathways that have been implicated in various diseases, disorders, or conditions. In particular, the methods contemplated in the instant invention comprise the use of a single agent drug that targets the right combination of multiple targets and achieving clinical efficacy.

In one embodiment, the method comprises administering to a patient in need thereof, a therapeutically effective amount of a single agent that simultaneously targets multiple kinases or kinase pathways.

In a preferred embodiment, the single agent targets one or more of the kinases or kinase pathways described in Section 5.3, below.

In particularly preferred embodiments, the single agent targets at least one member of the src kinase family of protein kinases. In other preferred embodiments, the single agent targets at least one member of the Rsk kinase family. In yet other preferred embodiments, the single agent is capable of targeting at least one member of the CDK family of protein kinases. In yet other preferred embodiments, the single agent targets at least one checkpoint kinase. In other preferred embodiments, the single agent targets at least one member of the MAPK kinase family of protein kinases. The invention also contemplates the use of single agents capable of targeting kinases including, but not limited to, ROCK-II, PRK2, PRAK, p70S6 kinase, or Aurora-A kinase.

In each of the above embodiments, it is also contemplated in the instant invention that the single agent simultaneously targets more than one kinase or kinase pathway, preferably more than two kinases or kinase pathways.

In one embodiment, the single agent simultaneously targets more than one kinase by directly modulating, regulating, or inhibiting each specific kinase's activity, expression, or fluction. In particular, the single agent simultaneously targets multiple kinases or kinase pathways and capable of directly and selectively targeting a particular kinase and/or kinase pathway while not affecting other kinases and/or pathways.

In one embodiment, the single agent is capable of simultaneously modulating, regulates, or inhibits multiple kinases or kinase pathways. In a specific embodiment, the single agent modulates the activity of, regulates, or inhibits the activity of multiple kinases. In another specific embodiment, the single agent modulates the activity of, regulates, or inhibits the expression of genes encoding multiple kinases. In yet another specific embodiment, the single agent modulates, regulates, or inhibits the function of multiple kinases. In an alternative embodiment, the single agent modulates the activity of, regulates, or inhibits the activity, expression, and/or finction of multiple kinases.

In one embodiment, the single agent modulates the activity of, regulates, or inhibits two or more kinases in the same kinase pathway. In an alternate embodiment, the single agent modulates, regulates, or inhibits multiple kinases in at least two different kinase pathways.

In another embodiment, the single agent modulates, regulates, or inhibits at least two kinases in the same kinase family. In an alternate embodiment, the single agent modulates, regulates, or inhibits multiple kinases in different kinase families.

In yet another embodiment, the single agent modulates, regulates, or inhibits corresponding kinases, their downstream targets, and/or their upstream targets.

In certain embodiments, the single agent modulates, regulates, or inhibits multiple genes encoding kinases that are abnormally expressed, abnormally activated, or mutated. In such embodiments, the kinases may be overexpressed or underexpressed or hyper-activated or under-activated/not activated at all.

In certain embodiments, the single agent targets a group of kinases that encompasses a plurality of kinase family members. In one embodiment, the single agent targets kinases that are cyclin nucleotide-regulated and phospholipid regulated kinases and ribosomal S6 kinases. Examples of kinases that would be a member of such a group are described herein, but also include, and are not limited to, protein kinase A (PKA), protein kinase G (PKG), and protein kinase C (PKC). In another embodiment, the single agent targets Ca²⁺/calmodulin kinases. In yet another embodiment of the invention, the single agent targets cyclin-dependent kinases. Examples of cyclin-dependent kinases are described herein, but also include, and are not limited to, cyclic dependent kinase (CDK1), mitogen activated protein kinase (MAPK/ERK) and glycogen synthase kinase (GSK3). In yet another embodiment of the invention, the single agent targets protein tyrosine kinases. Examples of tyrosine kinases are described herein, and include, and are not limited to, SRC and EGFR.

Any histidine kinase can be targeted using the methods of the invention, including, but not limited to, pyruvate dehydrogenase kinase isoenzyme 4 (PDK4), pyruvate dehydrogenase kinase isoenzyme 3 (PDK3), branched chain alpha-ketoacid dehydrogenase kinase (BCKDK) and pyruvate dehydrogenase kinase isoenzyme 1 (PDK1).

In certain other embodiments, the single agent targets serine/threonine kinases. In another embodiment, the single agent targets kinases that phosphorylate serine/threonine residues near arginine or lysine. In yet another embodiment, the single agent targets kinases that phosphorylate serine/threonine residues in proline rich domains. In another embodiment, the single agent targets tyrosine kinases of the receptor type. In yet another embodiment, the single agent targets tyrosine kinases of the non-receptor type. In certain embodiments, the single agent targets DNA dependent protein kinases (DNA-PK). In even another embodiment, the single agent targets kinases that are not identified in one of the groups, families or subfamilies described herein.

In a more particular embodiment of the invention, the single agent targets kinases that are related to the SRC family of kinases, preferably cSRC, YES, FYN and LCK.

In another more particular embodiment of the invention, the single agent targets kinases that are related to tyrosine kinases other than SRC related kinases. Preferably, said kinases are FES, LYN and SYK.

In another more particular embodiment, the single agent targets kinases that are related to the Rsk family of kinases, and preferably targets Rsk1, Rsk2 and Rsk3.

In another more particular embodiment, the single agent targets kinases that are related to the CDK family of kinases, preferably CDK1 /Cyclin B1, CDK2/Cyclin A, CDK3/Cyclin E, CDK5/p35, CDK6/Cyclin D3 and CDK7/Cyclin H/MAT1.

In another more particular embodiment, the single agent targets kinases that are related to the Checkpoint family of kinases, preferably CHK1 and CHK2.

In another more particular embodiment, the single agent targets kinases that are related to the MAPK family of kinases, preferably JNK1, MAPK1/ERK1, MAPK2/ERK2, MAPKAP-K5 and MEK1.

In another more particular embodiment, the single agent targets other kinases as identified herein in Section 5.3, including but not limited to ROCK-II, PRK2, PRAK, p70S6 kinase and Aurora A.

In certain embodiments, the single agent targets kinases or kinase pathways that interact with each other. In alternate embodiments, the single agent targets kinases or kinase pathways that do not interact with each other. In certain other embodiments, the single agent targets kinases or kinase pathways that are implicated in the same cellular process. In alternate embodiments, the single agent targets kinases or kinase pathways that are implicated in different cellular processes.

In a preferred embodiment, the single agent targets tyrosine kinase or tyrosine kinase pathways and is useful in the treatment of disorders such as diabetes, cancer, cell proliferative disorders, inflammation and obesity. See also Section 5.2.

In a preferred embodiment, the single agent targets at least human CDK1, CDK2, cSRC, Yes, MEK1 and Rsk1. In another preferred embodiment, the single agent inhibits the activity of at least three of CDK1, CDK2, cSRC, Yes, MEK1 and Rsk1 by at least 75% as compared to the activity of these kinases in equivalent conditions in the absence of the single agent. In another preferred embodiment, the single agent inhibits the activity of each of CDK1, CDK2, cSRC, Yes, MEK1 and Rsk1 by at least 90% as compared to the activity of these kinases in equivalent conditions in the absence of the single agent.

In another embodiment, the single agent shows antiproliferative activity in vitro in one or more drug resistant cell lines, where antiproliferative activity is demonstrated by a detectable reduction or diminution of the rate of proliferation of a particular proliferating cell line. In another embodiments, the single agent shows antiproliferative activity in vitro against of panel of one or more cancer cell lines. In yet another embodiment, the single agent inhibits a variety of kinases in in vitro kinase assays infra Section 6.

In another preferred embodiment, the single agent targets a cyclin dependent kinase or a cyclin dependent kinase pathway and is useful in the treatment of disorders such as cancer, hyperproliferative and immune disorders.

In a another preferred embodiment, the single agent targets a tyrosine kinase or a tyrosine kinase pathway and is useful in the treatment of disorders associated with increased or otherwise non-normal vascularization and angiogenesis. For example, the single agent is useful in the treatment of a cancer or tumor the growth of which is facilitated by increased vascularization or angiogenesis within and peripheral to the cancer or tumor.

In another embodiment, the single agent targets at least two, at least three, at least four, at least five or at least seven of the following kinase or kinase pathways: cSRC, Yes, Fyn, Lck, Fes, Lyn, Syk, Rsk, CDK1, CDK2, CDK3, CDK5, CDK6, CDK7, CHK1, CHK2, JNK1, MAPK1, MAPK2, MAPKAP-K5, MEK1, ROCKII, PRK2, PRAK, p70S6 and Aurora-A, and is useful for treating a disorder related to, for example, but not limited to, cancer or cell-proliferation; inappropriate or disease-related angiogenesis; cardiovascular disease; inflammation; insulin resistance, diabetes or obesity; a neurological disease; or infection by a microorganism.

In another embodiment, the single agent targets at least two, at least three, at least four, at least five or at least seven of the following kinase or kinase pathways: Yes, BMX, Syk, Eph, FGFR, RYK, MUSK, JAK1 and EGFR, and is useful for treating a disorder related to, for example, but not limited to, insulin resistance, diabetes or obesity; cancer, cell-proliferation and associated congenital syndromes; inflammation; inappropriate or disease-related angiogenesis; cardiovascular disease; or infection by a microorganism.

In another embodiment, the single agent targets at least two, at least three, at least four or at least five of the following kinase or kinase pathways: CDK, JNK, ERK, CDKL, ICK, CLK and DYRK, and is useful for treating a disorder related to, for example, but not limited to, cancer, cell-proliferation and associated congenital syndromes; insulin resistance, diabetes or obesity; a neurological disease; or infection by a microorganism.

In another embodiment, the single agent targets at least two, at least three, at least four, at least five or at least seven of the following kinase or kinase pathways: Src, Yes, Fyn, Lyn, Lck, Blk, Hck, Fgr, DYRK, and Yrk, and is useful for treating a disorder related to, for example, including but not limited to, cancer or hyperproliferation; immunity; inappropriate or disease-related angiogenesis; a neurological disease; cardiovascular disease; inflammation; or infection by a microorganism.

In another embodiment, the single agent targets at least two, at least three, at least four, at least five or at least seven of the following kinase or kinase pathways: MAPK, MAPK3, ERK2, MAPK7, JNK1, MAPK10, JNK3 alpha or MAPK14 and is useful for treating a disorder related to, for example, but not limited to insulin resistance, diabetes or obesity; inflammation; cardiovascular disease; inappropriate or disease-related angiogenesis; cancer, cell-proliferation or related congenital diseases; or infection by a microorganism.

In another embodiment, the single agent targets at least two, at least three, at least four, at least five or at least seven of the following kinase or kinase pathways: CHK1, CHK2, RSK1, RSK2, RSK3, Aurora-A, Aurora-B, ROCK1, ROCKII or p70S6K and is useful for treating a disorder related to, for example, but not limited to, insulin resistance, diabetes or obesity; inflammation; inappropriate or disease-related angiogenesis; cardiovascular disease; cancer, hyperproliferative disease or related congenital diseases; or infection by a microorganism.

In another embodiment, the single agent targets at least two or at least three of the following kinases or kinase pathways: pyruvate dehydrogenase kinase isoenzyme 4 (PDK4), pyruvate dehydrogenase kinase isoenzyme 3 (PDK3), branched chain alpha-ketoacid dehydrogenase kinase (BCKDK) or pyruvate dehydrogenase kinase isoenzyme 1 (PDK1) and is useful in treating, preventing, managing or ameliorating a disorder related to, for example, but not limited to, cancer, hyperproliferative disease or related congenital diseases; inflammation; angiogenesis; infection by a microorganism; cardiovascular disease; or insulin resistance, diabetes or obesity.

In another embodiment, the single agent targets an ephrin type receptor kinase and a neurotrophic type receptor kinase and is useful in the treatment of, for example, but not limited to, neurological disorders. In yet another preferred embodiment, the single agent targets a non-receptor tyrosine kinase and an IL-1 receptor associated kinase and is useful in the treatment of disorders that are related to, for example, but not limited to, hemopoiesis, immunology or angiogenesis. In yet another embodiment, the single agent targets at least two, at least three, or at least four of the following pathways: tyrosine kinase, phosphatidylinositol 3-kinase, JNK, IKK and PKC and is useful for treating a disorder related to, for example, but not limited to, insulin resistance, diabetes or obesity. In yet another embodiment, the single agent targets at least two at least three, at least four, at least five, at least seven of the following kinase or kinase pathways: ABL, EGFR, VEGFR, NGFR, PKC, PDGFR, CDK, MKK1, CHK1 and mTOR and is useful for treating a disorder related to, for example, but not limited to cancer or cell proliferation. In yet another embodiment, the single agent targets at least two, at least three or at least four of the following kinase or kinase pathways: PKC, Akt, PI-3 kinase, GSK3 and RTK and is useful for treating a disorder related to, for example, but not limited to, insulin resistance, diabetes or obesity.

The methods of the instant invention encompass the use of single agents that are compounds including but not limited to single agents that target more than one kinase or kinase pathway. In a preferred embodiment, the single agent can be the compound identified as CC001, CC002, CC004 or CC005.

The instant invention also contemplates the use of these single agents, also herein referred to as mixed kinases agents, alone (e.g., monotherapy) or in combination with one or more agents that specifically target a single kinase or kinase pathway and/or other therapies such as radiation therapy. For a more comprehensive list of other (adjuvant) therapies that may be used in combination with one or more single agents, see Section 5.4.3, below.

According to the methods of the present invention, identification of single agents that can simultaneously target, that is, modulate, regulate or inhibit multiple kinases or kinase pathways critically implicated in various diseases or disorders can be achieved by using kinase assays known in the art, for example, the assays described in Examples 4-84, below.

### 5.1 Patient Population

The present invention provides a method of treating an individual having a disease or condition associated with two or more kinases. As used herein, “individual,” “Patient” and the like may be a eukaryote, preferably a mammal, more preferably a human.

In one embodiment, the methods of the invention are useful in treating, managing, or preventing diseases or disorders in patients that have been refractory or resistant to single agents capable of targeting a single specific kinase or kinase pathway. It is also envisioned that the methods of the invention are useful in treating, managing, or preventing diseases or disorders in patients that have undergone, are currently undergoing, or may in the future undergo other treatments. For example, the compounds of the invention may be administered to individuals that have been administered other therapeutic agents, or that have undergone other therapies such as radiation or surgery.

The methods the invention are useful not only in untreated patients but are also useful in the treatment of patients partially or completely refractory to current standard and experimental therapies. In one embodiment, the invention provides therapeutic and prophylactic methods for the treatment or prevention of a disease, condition or disorder that has been shown to be or may be refractory or non-responsive to therapies other than those comprising administration of an agent capable of specifically targeting a single kinase or kinase pathway. In an alternate embodiment, the invention provides therapeutic and prophylactic methods for the treatment or prevention of a disease, condition or disorder that has been shown to be or may be refractory or non-responsive to therapies comprising administration of a single agent capable of specifically targeting a single kinase or kinase pathway.

In another embodiment, the methods of the invention are useful in treating, managing, or preventing diseases or disorders in patients that are currently receiving other therapies, such as, for example, anti-cancer therapies (e.g., chemotherapy, surgery, radiation therapy, antibody therapy, and the like); anti-inflammatory therapy (e.g., steroidal or non-steroidal anti-inflammatory drugs, antibody therapy, 0-agonists, cytokine therapy, and the like).

In yet another embodiment, the methods of the invention are useful in treating, managing, or preventing diseases or disorders in patients that have been determined to be pre-disposed to any disease condition, particularly to cancer, obesity, or inflammation-related disorders, or any of the disorders recited in Section 5.2.

### 5.2 Diseases and Disorders

The present invention encompasses therapies which involve administering one or more compounds to an animal, preferably a mammal, and most preferably a human, for preventing, treating, or ameliorating symptoms associated with a disease, disorder, or infection, associated with the activity or inactivity of one or more protein kinases. In a preferred embodiment, the invention relates to the prevention, treatment or amelioration of symptoms associated with a disease, disorder or infection, associated with the abnormal activity (e.g., abnormal upregulation or downregulation) of at least two protein kinases, at least three protein kinases, at least four protein kinases, at least five protein kinases, at least ten protein kinases or at least twenty protein kinases. In some embodiments, the methods of the invention are used in combination with one or more therapies such as, but not limited to, chemotherapies, radiation therapies, hormonal therapies, and/or biological therapies/immunotherapies.

The methods of the invention are useful in treating, preventing, managing or ameliorating a variety of diseases or disorders related to protein kinase activity, including, but not limited to, disorders related to the following: gene expression, cytoskeletal integrity, cell adhesion, cell cycle progression, differentiation and metabolism. Such diseases are controlled by the complex interplay of protein kinases and phosphatases, and associated malfunctions of cellular signaling have been linked to many diseases including cancer and diabetes. Sridhar et al., Pharmaceutical Research, 17(11) 1345-1353 (2000). Therapeutic strategies that target protein kinases and therefore regulate signal transduction have become the subject of intense research. For example, protein kinase C and tyrosine kinases have been implicated in certain types of cancer, diabetes and complications associated with diabetes. In addition protein kinase C isoforms have been implicated in cellular changes observed in the vascular complications of diabetes. Sridhar et al., Pharmaceutical Research, 17(11) 1345-1353 (2000); Chalfant et al., Mol. Endocrinol. 10:1273-1281 (1996). As a result of the complexity associated with protein kinase pathways, overlap exists between various protein kinases and a wide range of diseases or disorders. The methods of the invention relate to the treatment, management, prevention or amelioration of diseases associated with a protein kinase including, but not limited to, cancer, inflammatory disorders, diabetes, obesity, angiogenesis disorders and cardiovascular disorders.

In particular, the methods of the invention are useful for the prevention, treatment, management and/or amelioration of various diseases. By way of example, and not meant to limit, examples of the types of classes of disease that can be prevented, treated or managed include, but are not limited to, inflammatory conditions including, but not limited to: diabetes (such as Type II diabetes, Type I diabetes, diabetes insipidus, diabetes mellitus, maturity-onset diabetes, juvenile diabetes, insulin-dependant diabetes, non-insulin dependant diabetes, malnutrition-related diabetes, ketosis-prone diabetes or ketosis-resistant diabetes); nephropathy (such as glomerulonephritis or acute/chronic kidney failure); obesity (such as hereditary obesity, dietary obesity, hormone related obesity or obesity related to the administration of medication); hearing loss (such as that from otitis extema or acute otitis media); fibrosis related diseases (such as pulmonary interstitial fibrosis, renal fibrosis, cystic fibrosis, liver fibrosis, wound-healing or burn-healing, wherein the burn is a first-, second- or third-degree burn and/or a thermal, chemical or electrical burn); arthritis (such as rheumatoid arthritis, rheumatoid spondylitis, osteoarthritis or gout); an allergy; allergic rhinitis; acute respiratory distress syndrome; asthma; bronchitis; an inflammatory bowel disease (such as irritable bowel syndrome, mucous colitis, ulcerative colitis, Crohn's disease, gastritis, esophagitis, pancreatitis or peritonitis); or an autoimmune disease (such as scleroderma, systemic lupus erythematosus, myasthenia gravis, transplant rejection, endotoxin shock, sepsis, psoriasis, eczema, dermatitis or multiple sclerosis).

### 5.2.1 Proliferative Diseases

The methods of the invention can be used alone or in combination with other therapies known in the art to manage, treat, prevent, inhibit or reduce the incidence, appearance, growth or progression of a proliferative disease. In a specific embodiment, the methods of the invention, when administered alone or in combination with another cancer therapy known in the art, inhibits or reduces the incidence, appearance, growth or progression of a proliferative disease or condition, as measured by the number of affected cells, by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the growth of primary tumor or metastasis in absence of said methods of the invention.

In one embodiment, the proliferative disease is cancer. Accordingly, the invention provides methods of preventing or treating or managing cancer, inflammation, diabetes, obesity and other kinase-related disorders. In a specific embodiment, the methods of the invention, when administered alone or in combination with another cancer therapy known in the art, inhibits or reduces the growth of primary tumor or metastasis of cancerous cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the growth of primary tumor or metastasis in absence of said methods of the invention.

In various embodiments, the cancers treatable according to the invention may be cancers of the head, neck, eye, mouth, throat, esophagus, chest, bone, lung, colon, rectum, stomach, prostate, breast, ovaries, testicles or other reproductive organs, skin, thyroid, blood, lymph nodes, kidney, liver, pancreas, and brain or central nervous system.

Cancers and related disorders that can be treated or prevented by methods and compositions of the present invention include, but are not limited to, the following: Leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytoma and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including but not limited to, ocular melanoma such as iris melanoma, choroidal melanoma, and cilliary body melanoma, and retinoblastoma; vaginal cancers, including but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including but not limited to, squamous cell carcinoma, and adenocarcinoma; uterine cancers including but not limited to, endometrial carcinoma and uterine sarcoma; ovarian cancers including but not limited to, ovarian epithelial carcinoma, borderline tumor, germ cell tumor, and stromal tumor; esophageal cancers including but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including but not limited to, adenocarcinoma, flngating (polypoid), ulcerating, superficial spreading, diffuisely spreading, malignant lymphoma, liposarcoma, fibrosarcoma, and carcinosarcoma; colon cancers; rectal cancers; liver cancers including but not limited to hepatocellular carcinoma and hepatoblastoma, gallbladder cancers including but not limited to, adenocarcinoma; cholangiocarcinomas including but not limited to, pappillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including but not limited to, adenocarcinoma, leiomyosarcoma, and rhabdomyosarcoma; penal cancers; oral cancers including but not limited to, squamous cell carcinoma; basal cancers; salivary gland cancers including but not limited to, adenocarcinoma, mucoepidermoid carcinoma, and adenoidcystic carcinoma; pharynx cancers including but not limited to, squamous cell cancer, and verrucous; skin cancers including but not limited to, basal cell carcinoma, squamous cell carcinoma and melanoma, superficial spreading melanoma, nodular melanoma, lentigo malignant melanoma, acral lentiginous melanoma; kidney cancers including but not limited to, renal cell cancer, adenocarcinoma, hypemephroma, fibrosarcoma, transitional cell cancer (renal pelvis and/or uterer); Wilms' tumor; bladder cancers including but not limited to, transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, papillary adenocarcinomas, malignant sporadic melanoma, sporadic pancreatic cancer, Peutz-Jeghers syndrome, bladder, breast, colon, kidney, liver, lung, ovary, pancreas, stomach, cervix, thyroid and skin cancers; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions of the invention in the ovary, bladder, breast, colon, lung, skin, pancreas, or uterus. In other specific embodiments, sarcoma, melanoma, or leukemia is treated or prevented by the methods and compositions of the invention. (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

### 5.2.2 Inflammatory Disorders, Diabetes and Obesity

As described earlier, aberrant kinase activity has been associated with inflammatory disorders as well as with diabetes and its related effects. Sridhar et al., Pharmaceutical Research, 17(11)1345-1353 (2000). Indeed, a number of kinases have been implicated in cellular changes observed in the vascular complications of diabetes. Chalfant et al., Mol. Endocrinol. 10:1273-1281 (1996). Furthermore, as obesity is closely related with the insulin resistance found in type II diabetes, kinases that interfere with insulin action have been implicated in disorders related to obesity as well as diabetes. Hirosumi et al., Nature 420 333-336 (2002). Obesity and diabetes are also closely associated with the chronic inflammatory response that is mediated by kinases in various signal cascades. Id. at 333. As such, the methods of the invention relate to the management, treatment or prevention of inflammatory disorders, diabetes, obesity and their associated pathologies. Obesity treated according to the methods of the invention include hereditary obesity, dietary obesity, obesity related to the administration of medication or a course of therapy, obesity associated with diabetes, Examples of inflammatory and diabetes related disorders that may be managed, treated, prevented or ameliorated using the methods of the invention, include, but are not limited to, type I diabetes, juvenile diabetes, diabetes mellitus type II (NIDDM), noninsulin-dependent diabetes mellitus, maturity-onset diabetes dystrophia myotonica, malnutrition-related diabetes, ketosis-prone diabetes, ketoresistant diabetes, myotonic dystrophy 1, Steinert disease, liver glycogenosis, x-linked type I, hepatic phosphorylase kinase deficiency, phosphorylase kinase deficiency of liver, glycogenosis VIIIA, x-linked liver glycogenosis, phosphorylase kinase, liver glycogenosis x-linked type II, glycogen storage disease IX, glycogen storage disease VIII, lipoprotein lipase deficiency, 1pl deficiency, familial hyperchylomicronemia, hyperlipemia burger-grutz type, essential familial hyperlipemia, lipase D deficiency, type IA hyperlipoproteinemia, familial chylomicronemia, type I GM2-gangliosidosis, B variant GM2 gangliosidosis, hexosaminidase A deficiency, Tay-Sachs disease, pseudo-AB variant Tay-Sachs disease, mucoviscidosis, cystic fibrosis, galactose-1-phosphate uridylyltransferase deficiency, galt deficiency, classic galactosemia, chronic granulomatous disease, type I autosomal cytochrome-b-positive granulomatous disease, deficiency of neutrophil cytosol factor 1, deficiency of soluble oxidase component II, deficiency of soc2, deficiency of p47-phox, type I Gaucher disease, noncerebral juvenile gaucher disease, glucocerebrosidase deficiency, acid beta-glucosidase deficiency, hereditary sideroblastic anemia, hereditary iron-loading anemia, chronic granulomatous disease, X-linked cytochrome-b-negative granulomatous disease, asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, chronic inflammation resulting from chronic viral or bacteria infections, asthma, encephilitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral or bacteria infections.

Some autoimmune disorders are associated with inflammatory conditions. Thus, there is overlap between what is considered an autoimmune disorder and an inflammatory disorder. Therefore, some autoimmune disorders may also be characterized as inflammatory disorders. Examples of autoimmune disorders that may be managed, treated or prevented using the methods of the invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis.

### 5.2.3 Other Disorders—Angiogenesis, Cardiovascular, and Neurological Disorders

The invention relates to the management, treatment, prevention or amelioration of any diseases that is related to kinase activity. For example, any disease or disorder that is associated with kinase activity can be treated, managed, prevented or ameliorated using the methods of the invention. In one embodiment, the methods of the invention are used to treat angiogenesis related conditions that are related to kinase activity. In another embodiment, the methods of the invention are useful for the management, treatment, prevention or amelioration of diseases or disorders related to angiogenesis. IN other embodiments, the angiogenesis is preferably fundamental to a number of processes, such as growth, tissue repair, cancer, psoriasis, diabetic retinopathy and chronic inflammatory diseases in the lungs and joints.

Accordingly, in another embodiment, the methods of the invention are used to manage, treat, prevent or ameliorate a cardiovascular disease, including, but not limited to atherosclerosis, restenosis, left ventricular hypertrophy, myocardial infarction, chronic obstructive pulmonary disease or stroke. In yet another embodiment of the invention, the methods of the invention can be used to treat, manage, prevent or ameliorate diseases or disorders related to ischemia. Such diseases or disorders include, but are not limited to, ischemic conditions in the heart, kidney, liver or brain, and ischemia-reperfusion injury caused by, for example, transplant, surgical trauma, hypotension, thrombosis or trauma injury. In yet another embodiment, the methods of the invention are used to treat neurodegenerative disease, such as, but not limited to, epilepsy, Alzheimer's disease, Huntington's disease, Amyotrophic lateral sclerosis, peripheral neuropathies, spinal cord damage, AIDS dementia complex or Parkinson's disease.

The methods of the invention are also useful for managing, treating, preventing or ameliorating liver diseases. Such diseases, include, but are not limited to, hepatitis, alcohol-induced liver disease, toxin-induced liver disease, steatosis or sclerosis.

### 5.2.4. Diseases Involving Microorganisms Including Bacteria, Viruses and Fungi

The methods of the invention can be used to target kinases that are related to infections by a microorganisms. Such methods can be used to prevent infection of a host by a microorganism, such as, but not limited to, a bacteria, virus or fumgus. The methods of the invention can also be used to treat, prevent or ameliorate symptoms or conditions that are associated with infection by a microorganism, such as, but not limited to, a virus, bacteria or a fungus. Microorganisms, including viruses, that can infect an organism and that rely upon kinases for transmission, survival or homeostasis are known in the art. Such infectious agents that can be treated, prevented, managed or ameliorated using the methods of the invention include, but are not limited to, bacteria (e.g., gram positive bacteria, gram negative bacteria, aerobic bacteria, Spirochetes, Mycobacteria, Rickettsias, Chlamydias, etc.), parasites, fungi (e.g., Candida albicans, Aspergillus, etc.), viruses (e.g., DNA viruses, RNA viruses, etc.), or tumors. Viral infections that can be treated, prevented, managed or ameliorated include, but are not limited to, human immunodeficiency virus (HIV); hepatitis A virus, hepatitis B virus, hepatitis C virus, hepatitis D virus, or other hepatitis viruses; cytomegalovirus, herpes simplex virus-1 (-2, -3,-4,-5,-6), human papilloma viruses; Respiratory syncytial virus (RSV), Parainfluenza virus (PIV), Epstein Barr virus, Metapneumovirus (MPV) or any other viral infections.

### 5.3 Protein Kinases and Kinase Pathways

The methods of the invention encompass targeting any protein kinase, including, but not limited to, kinases that are known in the art and additional kinases that may be discovered. Such protein kinases include, but are not limited to, cyclin nucleotide-regulated and phospholipid-regulated kinases and ribosomal S6 kinases (herein also referred to as kinases of the group “AGC”), Ca²⁺/calmodulin kinases (herein referred to as kinases of the group “CaMK”), cyclin-dependent kinases (herein referred to as kinases of the group “CMGC”) and protein tyrosine kinases (herein referred to as kinases of the group “PTK”). These groups are meant only as examples of groups of kinases and are not meant to limit the possible groups that are embodied in the methods of the invention. In fact, the invention also relates to methods of targeting kinases that fall outside of the four major groups listed above.

Kinases that can be targeted using the methods of the invention can also be classified using particular features of their functional activity. For example, the methods of the invention encompass targeting a protein kinase that phosphorylates serine/threonine residues near arginine or lysine residues on substrate molecules, e.g., members of the AGC or CaMK groups. In another embodiment, the methods of the invention encompass targeting a protein kinase that phosphorylates serine/threonine residues in proline-rich domains on substrate molecules, e.g., members of the CMGC group. In yet another embodiment, the methods of the invention encompass targeting a protein receptor kinase that phosphorylates tyrosine residues, e.g., members of the PTK group. In even another embodiment, the methods of the invention encompass targeting a protein non-receptor kinase that phosphorylates tyrosine residues, e.g., members of the PTK group. Other kinases that can be targeted using the methods of the invention include dual specificity kinases, e.g., kinases that can phosphorylate serine/threonine and tyrosine residues.

In one embodiment, the kinases that are targeted by the methods of the invention include, but are not limited to, tyrosine kinases, both receptor and non-receptor tyrosine kinases. In a preferred embodiment, the tyrosine kinase is C-SRC, YES, FYN or LCK. In yet another preferred embodiment, the tyrosine kinase is FES, LYN or SYK. By way of example, and not meant to limit the possible kinases that may be targeted using the methods of the invention, other tyrosine kinases, include, but are not limited to, tyrosine-protein kinase (SYK), tyrosine-protein kinase (ZAP-70), protein tyrosine kinase 2 beta (PYK2), focal adhesion kinase 1 (FAK), B lymphocyte kinase (BLK), hemopoietic cell kinase (HCK), v-yes-1 Yamaguchi sarcoma viral related oncogene homolog (LYN), T cell-specific protein-tyrosine kinase (LCK), proto-oncogene tyrosine-protein kinase (YES), proto-oncogene tyrosine-protein kinase (SRC), proto-oncogene tyrosine-protein kinase (FYN), proto-oncogene tyrosine-protein kinase (FGR), proto-oncogene tyrosine-protein kinase (FER), proto-oncogene tyrosine-protein kinase (FES), C-SRC kinase, protein-tyrosine kinase (CYL), tyrosine protein kinase (CSK), megakaryocyte-associated tyrosine-protein kinase (CTK), tyrosine-protein kinase receptor (EPH), Ephrin type-A receptor 1, Ephrin type-A receptor 4 (EPHA4), Ephrin type-B receptor 3 (EPHB3), Ephrin type-A receptor 8 (EPHA8), neurotrophic tyrosine kinase receptor, type 1 (NTRK1), protein-tyrosine kinase (PTK2), syk-related tyrosine kinase (SRK), protein tyrosine kinase (CTK), tyro3 protein tyrosine kinase (TYRO3), bruton agammaglobulinemia tyrosine kinase (BTK), leukocyte tyrosine kinase (LTK), protein-tyrosine kinase (SYK), protein-tyrosine kinase (STY), tek tyrosine kinase (TEK), elk-related tyrosine kinase (ERK), tyrosine kinase with immunoglobulin and egf factor homology domains (TIE), protein tyrosine kinase (TKF), neurotrophic tyrosine kinase, receptor, type 3 (NTRK3), mixed-lineage protein kinase-3 (MLK3), protein kinase, mitogen-activated 4 (PRKM4), protein kinase, mitogen-activated 1 (PRKM1), protein tyrosine kinase (PTK7), protein tyrosine kinase (EEK), minibrain (drosophila) homolog (MNBH), bone marrow kinase, x-linked (BMX), eph-like tyrosine kinase 1 (ETK1), macrophage stimulating 1 receptor (MST1R), btk-associated protein, 135 kd, lymphocyte-specific protein tyrosine kinase (LCK), fibroblast growth factor receptor-2 (FGFR2), protein tyrosine kinase-3 (TYK3), protein tyrosine kinase (TXK), tec protein tyrosine kinase (TEC), protein tyrosine kinase-2 (TYK2), eph-related receptor tyrosine kinase ligand 1 (EPLG1), t-cell tyrosine kinase (EMT), eph tyrosine kinase 1 (EPHT1), zona pellucida receptor tyrosine kinase, 95 kd (ZRK), protein kinase, mitogen-activated, kinase 1 (PRKMK1), eph tyrosine kinase 3 (EPHT3), growth arrest-specific gene-6 (GAS6), kinase insert domain receptor (KDR), axl receptor tyrosine kinase (AXL), fibroblast growth factor receptor-1 (FGFR1), v-erb-b2 avian erythroblastic leukemia viral oncogene homolog 2 (ERBB2), fms-like tyrosine kinase-3 (FLT3), neuroepithelial tyrosine kinase (NEP), neurotrophic tyrosine kinase receptor-related 3 (NTRKR3), eph-related receptor tyrosine kinase ligand 5 (EPLG5), neurotrophic tyrosine kinase, receptor, type 2 (NTRK2), receptor-like tyrosine kinase (RYK), tyrosine kinase, b-lymphocyte specific (BLK), eph tyrosine kinase 2 (EPHT2), eph-related receptor tyrosine kinase ligand 2 (EPLG2), glycogen storage disease VIII, eph-related receptor tyrosine kinase ligand 7 (EPLG7), janus kinase 1 (JAK1), fms-related tyrosine kinase-l (FLT1), protein kinase, camp-dependent, regulatory, type I, alpha (PRKAR1A), wee-i tyrosine kinase (WEE1), eph-like tyrosine kinase 2 (ETK2), receptor tyrosine kinase musk, insulin receptor (INSR), janus kinase 3 (JAK3), fms-related tyrosine kinase-3 ligand protein kinase c, beta 1 (PRKCB1), tyrosine kinase-type cell surface receptor (HER3), janus kinase 2 (JAK2), lim domain kinase 1 (LIMK1), dual specificity phosphatase 1 (DUSP1), hemopoietic cell kinase (HCK), tyrosine 3-monooxygenase/tryptophan 5-monooxygenase activation protein, eta polypeptide (YWHAH), ret proto-oncogene (RET), tyrosine 3-monooxygenase/tryptophan 5-monooxygenase activation protein, zeta polypeptide (YWHAZ), tyrosine 3-monooxygenase/tryptophan 5-monooxygenase activation protein, beta polypeptide (YWHAB), hepatoma transmembrane kinase (HTK), map kinase kinase 6, phosphatidylinositol 3-kinase, catalytic, alpha polypeptide (PIK3CA), cyclin-dependent kinase inhibitor 3 (CDKN3), diacylglycerol kinase, delta, 130 kd, protein-tyrosine phosphatase, nonreceptor type, 13 (PTPN13), abelson murine leukemia viral oncogene homolog 1 (ABLI), diacylglycerol kinase, alpha (DAGK1), focal adhesion kinase 2, epithelial discoidin domain receptor 1 (EDDR1), anaplastic lymphoma kinase (ALK), phosphatidylinositol 3-kinase, catalytic, gamma polypeptide (PIK3CG), phosphatidylinositol 3-kinase regulatory subunit, (PIK3R1), eph homology kinase-1 (EHK1), v-kit hardy-zuckerman 4 feline sarcoma viral oncogene homolog (KIT), fibroblast growth factor receptor-3 (FGFR3), vascular endothelial growth factor c (VEGFC), epidermal growth factor receptor (EGFR), oncogene (TRK), growth factor receptor-bound protein-7 (GRB7), ras p21 protein activator (RASA2), met proto-oncogene (MET), src-like adapter (SLA), vascular endothelial growth factor (VEGF), vascular endothelial growth factor receptor (VEGFR), nerve growth factor receptor (NGFR), platelet derived growth factor receptor (PDGFR), platelet derived growth factor receptor beta (PDGFRB), dual-specificity tyrosine-(Y)-phosphorylation regulated kinase 2 (DYRK2), dual-specificity tyrosine-(Y)-phosphorylation regulated kinase 3 (DYRK3), dual-specificity tyrosine-(Y)-phosphorylation regulated kinase 4 (DYRK4), dual-specificity tyrosine-(Y)-phosphorylation regulated kinase 1A (DYRK1A), dual-specificity tyrosine-(Y)-phosphorylation regulated kinase 1B (DYRK1B), CDC-like kinase 1 (CLK1), protein tyrosine kinase STY, CDC-like kinase 4 (CLK4), CDC-like kinase 2 (CLK2) and CDC-like kinase 3 (CLK3).

In another embodiment, the methods of the invention are used to target serine/threonine kinases. By way of example, and not meant to limit the possible kinases that may be targeted using the methods of the invention, such serine/threonine kinases or related molecules include, but are not limited to, cyclin-dependent kinase 7 (CDK7), rac serine/threonine protein kinase, serine-threonine protein kinase n (PKN), serine/threonine protein kinase 2 (STK2), zipper protein kinase (ZPK), protein-tyrosine kinase (STY), bruton agammaglobulinemia tyrosine kinase (BTK), mkn28 kinase, protein kinase, x-linked (PRKX), elk-related tyrosine kinase (ERK), ribosomal protein s6 kinase, 90 kd, polypeptide 3 (RPS6KA3), glycogen storage disease VIII, death-associated protein kinase 1 (DAPK1), pctaire protein kinase 1 (PCTK1), protein kinase, interferon-inducible double-stranded rna (PRKR), activin a receptor, type II-like kinase 1 (ACVRLK1), protein kinase, camp-dependent, catalytic, alpha (PRKACA), protein kinase, y-linked (PRKY), G protein-coupled receptor kinase 2 (GPRK21), protein kinase c, theta form (PRKCQ), lim domain kinase 1 (LIMK1), phosphoglycerate kinase 1 PGKI), lim domain kinase 2 (LIMK2), c-jun kinase, activin a receptor, type II-like kinase 2 (ACVRLK2), janus kinase 1 (JAK1), elkl motif kinase (EMK1), male germ cell-associated kinase (MAK), casein kinase 2, alpha-prime subunit (CSNK2A2), casein kinase 2, beta polypeptide (CSNK2B), casein kinase 2, alpha 1 polypeptide (CSNK2A1), ret proto-oncogene (RET), hematopoietic progenitor kinase 1, conserved helix-loop-helix ubiquitous kinase (CHUK), casein kinase 1, delta (CSNK1D), casein kinase 1, epsilon (CSNK1E), v-akt murine thymoma viral oncogene homolog 1 (AKT1), tumor protein p53 (TP53), protein phosphatase 1, regulatory (inhibitor) subunit 2 (PPP1R2), oncogene pim-1 (PIM1), transforming growth factor-beta receptor, type II (TGFBR2), transforming growth factor-beta receptor, type I (TGFBR1), v-raf murine sarcoma viral oncogene homolog b1 (BRAF), bone morphogenetic receptor type II (BMPR2), v-raf murine sarcoma 3611 viral oncogene homolog 1 (ARAF1), v-raf murine sarcoma 3611 viral oncogene homolog 2 (ARAF2), protein kinase C (PKC), v-kit hardy-zuckerman 4 feline sarcoma viral oncogene homolog (KIT) and c-KIT receptor (KITR).

In one embodiment, the kinases that are targeted using the methods of the invention include kinases that are cyclin dependent or from the CDK family of kinases. While the invention embodies the modulation of any member of the CDK family of kinases, in a particular embodiment, the CDK is CDK1/Cyclin B1, CDK2/Cyclin A, CDK3/Cyclin E, CDK5/p35, CDK6/cyclin D3 or CDK7/Cyclin H/MAT1. By way of example, and not meant to limit the possible kinases that may be targeted using the methods of the invention, other members of the CDK family of kinases that may be targeted using the methods of the invention, include, but are not limited to, cyclin dependent kinase 2 (CDK2), cyclin dependent kinase 7 (CDK7), CDK-activating kinase (CAK), TFIIH basal transcription factor complex kinase subunit, 39 kDa protein kinase, STK1, CAK1, cyclin dependent kinase 6 (CDK6), cell division control 2 protein (CDC2), p34 protein kinase, cyclin dependent kinase 1 (CDK1), cell division protein kinase 1, cell division protein kinase 2 (CDK2), cyclin dependent protein kinase 2, p33 protein kinase, cell division protein kinase 3 (CDK3), cyclin dependent protein kinase 3, cell division protein kinase 5 (CDK5), cyclin dependent protein kinase 5, tau protein kinase II (TPKII), serine/threonine protein kinase (PSSALRE), serine/threonine-protein kinase PCTAIRE-1 (PCTAIRE1), serine/threonine-protein kinase (PCTAIRE-2), serine/threonine protein kinase PFTAIRE-1 (PFTAIRE1), serine/threonine-protein kinase ALS2CR7 (PFTAIRE2), cell division protein kinase 4 (CDK4), cyclin-dependent kinase 4, PSK-J3, cell division protein kinase 6, serine/threonine protein kinase (PLSTIRE), cell division protein kinase 10 (CDK10), cyclin dependent protein kinase 10, serine/threonine-protein kinase (PISSLRE), serine-threonine kinase CDC2L 1 (PITSLRE), galactosyltransferase associated protein kinase p58/GTA, cell division cycle 2-like 1 (CLK-1), CDK11, p58 CLK-1, cell division protein kinase 9 (CDK9), cyclin dependent protein kinase 9, serine/threonine-protein kinase PITALRE, C-2K, cell division cycle 2-like 5 (cholinesterase-related cell division controller) (CHED), CDC2L, cell division cycle 2-related protein kinase 7 (CRK7), CDC2-related protein kinase 7, cell division protein kinase 8 (CDK8), protein kinase K35, cyclin-dependent kinase-like 1 (CDC2-related kinase) (CDKL1), CDC2-related kinase 1, serine/threonine protein kinase, cyclin-dependent kinase-like 2 (CDC2-related kinase) (CDKL2), CDC2-related kinase, p56 protein kinase, cyclin-dependent kinase-like 3 (CDKL3), cyclin-dependent kinase-like 5 (CDKL5), glycogen synthase kinase 3 alpha (GSK3alpha), glycogen synthase kinase 3 beta (GSK3beta) and intestinal cell (MAK-like) kinase (ICK). Many of these cyclin dependent kinases have been found to be involved in cellular signaling pathways involved in various pathological conditions including, but not limited to, cancer and hyperproliferative disorders and immune disorders.

In one embodiment of the invention, the kinases that are targeted using the methods of the invention include MAP kinases. While the invention embodies the modulation of any member of the MAP family of kinases, in a preferred embodiment, the MAP kinases are JNK1, MAPK1I/ERK1, MAPK2/ERK2, MAPKAP-K5 or MEK1. By way of example, and not meant to limit the possible kinases that may be targeted using the methods of the invention, other members of the MAP kinase family of kinases that may be targeted using the methods of the invention, include, but are not limited to, mitogen-activated protein kinase 3 (MAPK3), p44erk1, p44mapk, mitogen-activated protein kinase 3 (MAP kinase 3; p44), ERK1, PRKM3, P44ERK1, P44MAPK, mitogen-activated protein kinase 1 (MAPK1), mitogen-activated protein kinase kinase 1 (MEK1), MAP2K1protein tyrosine kinase ERK2, mitogen-activated protein kinase 2, extracellular signal-regulated kinase 2, protein tyrosine kinase ERK2, mitogen-activated protein kinase 2, extracellular signal-regulated kinase 2, ERK, p38, p40, p41, ERK2, ERT1, MAPK2, PRKM1, PRKM2, P42MAPK, p41mapk, mitogen-activated protein kinase 7 (MAPK7), BMK1 kinase, extracellular-signal-regulated kinase 5, BMK1, ERK4, ERK5, PRKM7, nemo-like kinase (NLK), likely ortholog of mouse nemo like kinase, mitogen-activated protein kinase 8 (MAPK8), protein kinase JNK1, JNK1 beta protein kinase, JNK 1 alpha protein kinase, c-Jun N-terminal kinase 1, stress-activated protein kinase JNK1, JNK, JNK1, PRKM8, SAPK1, JNK1A2, JNK21B1/2, mitogen-activated protein kinase 10 (MAPK10), c-Jun kinase 3, JNK3 alpha protein kinase, c-Jun N-terminal kinase 3, stress activated protein kinase JNK3, stress activated protein kinase beta, mitogen-activated protein kinase 9 (MAPK9), MAP kinase 9, c-Jun kinase 2, c-Jun N-terminal kinase 2, stress-activated protein kinase JNK2, JNK2, JNK2A, JNK2B, PRKM9, JNK-55, JNK2BETA, p54aSAPK, JNK2ALPHA, mitogen-activated protein kinase 14 (MAPK14), p38 MAP kinase, MAP kinase Mxi2, Csaids binding protein, MAX-interacting protein 2, stress-activated protein kinase 2A, p38 mitogen activated protein kinase, cytokine suppressive anti-inflammatory drug binding protein, RK, p38, EXIP, Mxi2, CSBP1, CSBP2, CSPB1, PRKM14, PRKM15, SAPK2A, p38ALPHA, mitogen-activated protein kinase 11 (MAPK11), stress-activated protein kinase-2, stress-activated protein kinase-2b, mitogen-activated protein kinase p38-2, mitogen-activated protein kinase p38beta, P38B, SAPK2, p38-2, PRKM11, SAPK2B, p38Beta, P38BETA2, mitogen-activated protein kinase 13 (MAPK13), stress-activated protein kinase 4, mitogen-activated protein kinase p38 delta, SAPK4, PRKM13, p38delta, mitogen-activated protein kinase 12 (MAPK12), p38gamma, stress-activated protein kinase 3, mitogen-activated protein kinase 3, ERK3, ERK6, SAPK3, PRKM12, SAPK-3, P38GAMMA, mitogen-activated protein kinase 6 (MAPK6), MAP kinase isoform p97, mitogen-activated 5 protein kinase, mitogen-activated 6 protein kinase, extracellular signal-regulated kinase 3, extracellular signal-regulated kinase, p97, ERK3, PRKM6, p97MAPK, mitogen-activated protein kinase 4 (MAPK4), Erk3-related protein kinase, mitogen-activated 4 protein kinase (MAP kinase 4; p63), PRKM4, p63MAPK, ERK3-RELATED and Extracellular signal-regulated kinase 8 (ERK7).

In one embodiment, the kinases that are targeted using the methods of the invention include Rsk kinases. While the invention embodies the modulation of any member of the Rsk family of kinases, in a more preferred embodiment, the Rsk kinase is Rsk1, Rsk2 or Rsk3. By way of example, and not meant to limit the possible kinases that may be targeted using the methods of the invention, other members of the RSK family of kinases that may be targeted using the methods of the invention, include, but are not limited to, ribosomal protein S6 kinase-like 1 (RskL2), ribosomal protein S6 kinase, 52kDa, polypeptide 1 (RskL2), ribosomal protein S6 kinase, 90 kDa, polypeptide 2 (Rsk3), ribosomal protein S6 kinase, 90 kDa, polypeptide 6 (Rsk4), ribosomal protein S6 kinase, 90 kDa, polypeptide 3 (Rsk2), ribosomal protein S6 kinase, 90 kDa, polypeptide 1 (Rsk1/p90Rsk), p70 ribosomal S6 kinase beta, ribosomal protein S6 kinase, 70 kD, polypeptide 2 (p70S6K,), serine/threonine kinase 14 alpha, ribosomal protein S6 kinase, 70 kD, polypeptide 1 (p70S6K), ribosomal protein S6 kinase, 90 kD, polypeptide 4, ribosomal protein kinase B, mitogen and stress-activated protein kinase 2 (MSK2), mitogen- and stress-activated protein kinase 1 and ribosomal protein S6 kinase, 90 kD, polypeptide 5 (MSK1).

In one embodiment of the invention, the kinases that are targeted include kinases from the CHK family. While the invention embodies the modulation of any member of the CHK family, in a preferred embodiment, the CHK kinase is CHK1 or CHK2. By way of example, and not meant to limit the possible kinases that may be targeted using the methods of the invention, other members of the checkpoint family that may be targeted using the methods of the invention, include, but are not limited to, serine/threonine-protein kinase (CHK1), serine/threonine kinase 11 (LKB1), PAS-serine/threonine kinase (PASK), Serine/threonine-protein kinase PIM-2, proto-oncogene serine/threonine-protein kinase PIM-1 and proto-oncogene serine/threonine-protein kinase PIM-3.

Other families of kinases that can be targeted using the methods of the invention include, but are not limited to, those that are related to Rho-associated coiled-coil containing protein kinase p160ROCK (ROCK1), Rho kinase (ROCK2), mitogen-activated protein kinase-activated protein kinase 5 (PRAK), ribosomal protein S6 kinase (p70S6α), Ribosomal protein S6 kinase beta 2 (p70S6β), serine/threonine protein kinase 15 (Aurora A) and serine/threonine protein kinase 12 (Aurora B).

### 5.4 Therapeutic/Prophylactic Protocols and Regimans

### 5.4.1 Methods

The present invention encompasses the use of one or more single agents in the treatment of a disease, disorder or condition associated, at least in part, with the activity of at least two different kinases.

In one embodiment, the invention provides a method of modulating the activity of a plurality of kinases, comprising contacting said plurality of kinases with a single agent in an amount sufficient to cause a detectable change in the activity of said plurality of kinases, wherein said plurality of kinases is at least two of CDK1, CDK2, cSRC, Yes, MEK1, Rsk1. In a preferred embodiment, the invention provides a method of inhibiting the activity of at least three of the kinases CDK1, CDK2, cSRC, Yes, MEK1 and Rsk1 by at least 75% as compared to the activity of said kinases in equivalent conditions in the absence of the single agent, comprising contacting at least three of said kinases with a single agent. In a specific embodiment, said single agent is CC001 or CC004. In another preferred embodiment, the invention provides a method of inhibiting the activity of each of CDK1, CDK2, cSRC, Yes, MEK1 and Rsk1 by at least 90% as compared to the activity of these kinases in equivalent conditions in the absence of the single agent, comprising contacting at least three of said kinases with a single agent. In a specific embodiment, said single agent is CC001.

In another embodiment, the single agent shows antiproliferative activity in vitro in one or more drug resistant cell lines, where antiproliferative activity is demonstrated by a detectable reduction or diminution of the rate of proliferation of a particular proliferating cell line. In another embodiments, the single agent shows antiproliferative activity in vitro against of panel of one or more cancer cell lines. In yet another embodiment, the single agent inhibits a variety of kinases in in vitro kinase assays infra Section 6.

In another preferred embodiment, the single agent targets a cyclin dependent kinase or a cyclin dependent kinase pathway and is useful in the treatment of disorders such as cancer, hyperproliferative and immune disorders.

In a another preferred embodiment, the single agent targets a tyrosine kinase or a tyrosine kinase pathway and is useful in the treatment of disorders associated with increased or otherwise non-normal vascularization and angiogenesis. For example, the single agent is useful in the treatment of a cancer or tumor the growth of which is facilitated by increased vascularization or angiogenesis within and peripheral to the cancer or tumor.

In another embodiment, the invention provides a method of modulating the activity of a plurality of kinases relative to the activity of said kinases in equivalent conditions in the absence of said single agent, comprising contacting said plurality of kinases with a single agent in a concentration sufficient to detectably modulate the activity of said plurality of kinases, wherein said plurality of kinases includes two or more of cSRC, Yes, Fyn, Lck, Fes, Lyn, Syk, Rsk, CDK1, CDK2, CDK3, CDK5, CDK6, CDK7, CHK1, CHK2, JNK1, MAPK1, MAPK2, MAPKAP-K5, MEK1, ROCKII, PRK2, PRAK, p70S6 or Aurora-A. In a specific embodiment, said contacting is performed in vivo in an individual suffering from a condition at a concentration sufficient to treat said condition, wherein said condition is cancer or cell-proliferation; inappropriate or disease-related angiogenesis; cardiovascular disease; inflammation; insulin resistance, diabetes or obesity; a neurological disease; or infection by a microorganism. In another embodiment, the invention provides a method of treating an individual suffering from a condition, comprising administering to said individual a single agent in an amount sufficient to modulate the activity of two or more of cSRC, Yes, Fyn, Lck, Fes, Lyn, Syk, Rsk, CDK1, CDK2, CDK3, CDK5, CDK6, CDK7, CHK1, CHK2, JNK1, MAPK1, MAPK2, MAPKAP-K5, MEK1, ROCKII, PRK2, PRAK, p70S6 or Aurora-A, and wherein said condition is cancer or cell-proliferation; inappropriate or disease-related angiogenesis; cardiovascular disease; inflammation; insulin resistance, diabetes or obesity; a neurological disease; or infection by a microorganism.

In another embodiment, the invention provides a method of modulating the activity of a plurality of kinases relative to the activity of said kinases in equivalent conditions in the absence of said single agent, comprising contacting said plurality of kinases with a single agent in a concentration sufficient to detectably modulate the activity of said plurality of kinases, wherein said plurality of kinases includes two or more of Yes, BMX, Syk, Eph, FGFR, RYK, MUSK, JAK1 or EGFR. In a specific embodiment, said contacting is performed in vivo in an individual suffering from a condition at a concentration sufficient to treat said condition, wherein said condition is insulin resistance, diabetes or obesity; cancer, cell-proliferation or associated congenital syndromes; inflammation; inappropriate or disease-related angiogenesis; cardiovascular disease; or infection by a microorganism. In another embodiment, the invention provides a method of treating an individual suffering from a condition, comprising administering to said individual a single agent in an amount sufficient to modulate the activity of two or more of Yes, BMX, Syk, Eph, FGFR, RYK, MUSK, JAK1 and EGFR, and wherein said condition is insulin resistance, diabetes or obesity; cancer, cell-proliferation or associated congenital syndromes; inflammation; inappropriate or disease-related angiogenesis; cardiovascular disease; or infection by a microorganism.

In another embodiment, the invention provides a method of modulating the activity of a plurality of kinases relative to the activity of said kinases in equivalent conditions in the absence of said single agent, comprising contacting said plurality of kinases with a single agent in a concentration sufficient to detectably modulate the activity of said plurality of kinases, wherein said plurality of kinases includes two or more of CDK, JNK, ERK, CDKL, ICK, CLK and DYRK. In a specific embodiment, said contacting is performed in vivo in an individual suffering from a condition at a concentration sufficient to treat said condition, wherein said condition is cancer, cell-proliferation or associated congenital syndromes; insulin resistance, diabetes or obesity; a neurological disease; or infection by a microorganism. In another embodiment, the invention provides a method of treating an individual suffering from a condition, comprising administering to said individual a single agent in an amount sufficient to modulate the activity of two or more of CDK, JNK, ERK, CDKL, ICK, CLK and DYRK, and wherein said condition is cancer, cell-proliferation or associated congenital syndromes; insulin resistance, diabetes or obesity; a neurological disease; or infection by a microorganism.

In another embodiment, the invention provides a method of modulating the activity of a plurality of kinases relative to the activity of said kinases in equivalent conditions in the absence of said single agent, comprising contacting said plurality of kinases with a single agent in a concentration sufficient to detectably modulate the activity of said plurality of kinases, wherein said plurality of kinases includes two or more of Src, Yes, Fyn, Lyn, Lck, Blk, Hck, Fgr, DYRK, and Yrk. In a specific embodiment, said contacting is performed in vivo in an individual suffering from a condition at a concentration sufficient to treat said condition, wherein said condition is cancer or hyperproliferation; immunity; inappropriate or disease-related angiogenesis; a neurological disease; cardiovascular disease; inflammation; or infection by a microorganism. In another embodiment, the invention provides a method of treating an individual suffering from a condition, comprising administering to said individual a single agent in an amount sufficient to modulate the activity of two or more of Src, Yes, Fyn, Lyn, Lck, Blk, Hck, Fgr, DYRK, and Yrk, and wherein said condition is cancer or hyperproliferation; immunity; inappropriate or disease-related angiogenesis; a neurological disease; cardiovascular disease; inflammation; or infection by a microorganism.

In another embodiment, the invention provides a method of modulating the activity of a plurality of kinases relative to the activity of said kinases in equivalent conditions in the absence of said single agent, comprising contacting said plurality of kinases with a single agent in a concentration sufficient to detectably modulate the activity of said plurality of kinases, wherein said plurality of kinases includes two or more of MAPK, MAPK3, ERK2, MAPK7, JNK1, MAPK10, JNK3 alpha or MAPK14. In a specific embodiment, said contacting is performed in vivo in an individual suffering from a condition at a concentration sufficient to treat said condition, wherein said condition is insulin resistance, diabetes or obesity; inflammation; cardiovascular disease; inappropriate or disease-related angiogenesis; cancer, cell-proliferation or related congenital diseases; or infection by a microorganism. In another embodiment, the invention provides a method of treating an individual suffering from a condition, comprising administering to said individual a single agent in an amount sufficient to modulate the activity of two or more of MAPK, MAPK3, ERK2, MAPK7, JNK1, MAPK10, JNK3 alpha or MAPK14, wherein said condition is insulin resistance, diabetes or obesity; inflammation; cardiovascular disease; inappropriate or disease-related angiogenesis; cancer, cell-proliferation or related congenital diseases; or infection by a microorganism.

In another embodiment, the invention provides a method of modulating the activity of a plurality of kinases relative to the activity of said kinases in equivalent conditions in the absence of said single agent, comprising contacting said plurality of kinases with a single agent in a concentration sufficient to detectably modulate the activity of said plurality of kinases, wherein said plurality of kinases includes two or more of CHK1, CHK2, RSK1, RSK2, RSK3, Aurora-A, Aurora-B, ROCK1, ROCKII or p70S6K. In a specific embodiment, said contacting is performed in vivo in an individual suffering from a condition at a concentration sufficient to treat said condition, wherein said condition is insulin resistance, diabetes or obesity; inflammation; inappropriate or disease-related angiogenesis; cardiovascular disease; cancer, hyperproliferative disease or related congenital diseases; or infection by a microorganism. In another embodiment, the invention provides a method of treating an individual suffering from a condition, comprising administering to said individual a single agent in an amount sufficient to modulate the activity of two or more of CHKl, CHK2, RSK1, RSK2, RSK3, Aurora-A, Aurora-B, ROCK1, ROCKII or p70S6K wherein said condition is insulin resistance, diabetes or obesity; inflammation; inappropriate or disease-related angiogenesis; cardiovascular disease; cancer, hyperproliferative disease or related congenital diseases; or infection by a microorganism.

In another embodiment, the invention provides a method of modulating the activity of a plurality of kinases relative to the activity of said kinases in equivalent conditions in the absence of said single agent, comprising contacting said plurality of kinases with a single agent in a concentration sufficient to detectably modulate the activity of said plurality of kinases, wherein said plurality of kinases includes two or more of pyruvate dehydrogenase kinase isoenzyme 4 (PDK4), pyruvate dehydrogenase kinase isoenzyme 3 (PDK3), branched chain alpha-ketoacid dehydrogenase kinase (BCKDK) or pyruvate dehydrogenase kinase isoenzyme 1 (PDK1). In a specific embodiment, said contacting is performed in vivo in an individual suffering from a condition at a concentration sufficient to treat said condition, wherein said condition is cancer, hyperproliferative disease or related congenital diseases; inflammation; angiogenesis; infection by a microorganism; cardiovascular disease; or insulin resistance, diabetes or obesity. In another embodiment, the invention provides a method of treating an individual suffering from a condition, comprising administering to said individual a single agent in an amount sufficient to modulate the activity of two or more of pyruvate dehydrogenase kinase isoenzyme 4 (PDK4), pyruvate dehydrogenase kinase isoenzyme 3 (PDK3), branched chain alpha-ketoacid dehydrogenase kinase (BCKDK) or pyruvate dehydrogenase kinase isoenzyme 1 (PDK1), wherein said condition is cancer, hyperproliferative disease or related congenital diseases; inflammation; angiogenesis; infection by a microorganism; cardiovascular disease; or insulin resistance, diabetes or obesity.

In another embodiment, the invention provides a method of treating an individual suffering from a condition, comprising administering to said individual a single agent in an amount sufficient to modulate the activity of two or more of CDK1, CDK2, cSrc, Yes, Rsk1 or MEK1, wherein said condition is cancer or a proliferative disorder.

In another embodiment, the single agent targets an ephrin type receptor kinase and a neurotrophic type receptor kinase and is useful in the treatment of, for example, but not limited to, neurological disorders. In yet another preferred embodiment, the single agent targets a non-receptor tyrosine kinase and an IL-1 receptor associated kinase and is useful in the treatment of disorders that are related to, for example, but not limited to, hemopoiesis, immunology or angiogenesis. In yet another embodiment, the single agent targets at least two, at least three, or at least four of the following pathways: tyrosine kinase, phosphatidylinositol 3-kinase, JNK, IKK and PKC and is useful for treating a disorder related to, for example, but not limited to, insulin resistance, diabetes or obesity. In yet another embodiment, the single agent targets at least two at least three, at least four, at least five, at least seven of the following kinase or kinase pathways: ABL, EGFR, VEGFR, NGFR, PKC, PDGFR, CDK, MKK1, CHK1 and mTOR and is useful for treating a disorder related to, for example, but not limited to cancer or cell proliferation. In yet another embodiment, the single agent targets at least two, at least three or at least four of the following kinase or kinase pathways: PKC, Akt, PI-3 kinase, GSK3 and RTK and is useful for treating a disorder related to, for example, but not limited to, insulin resistance, diabetes or obesity.

In another embodiment, the invention provides a method of inhibiting the activity of a plurality of kinases, comprising contacting said plurality of kinases with a single agent, wherein the activity of at least two of said plurality of kinases is inhibited detectably, or is inhibited by at least 20%, 25%, 30%, 35%, 40%, 45%, 50%, 55%, 60%, 65%, 70%, 75%, 80%, 85%, 90% or at least 95% by said single agent, relative to the activity of said at least two of said plurality of said kinases under equivalent conditions in the absence of said single agent. In a more specific embodiment, said plurality of kinases comprises CDK1, CHK2, PRK2 and ROCK-II, and said single agent inhibits said CDK1, CHK2, PRK2 and ROCK-II by at least 90%, relative to the activity of said at least two of said plurality of said kinases under equivalent conditions in the absence of said single agent, in an in vitro kinase assay in which said single agent is present at a concentration of about 3 μM. In another specific embodiment, said single agent inhibits at least 7 of the kinases listed in Table 2 by 90% or more, relative to the activity of said at least 7 of the kinases listed in Table 2 under equivalent conditions in the absence of said single agent, in an in vitro kinase assay in which said single agent is present at a concentration of about 3μM. In another specific embodiment, said single agent inhibits at least 12 of the kinases listed in Table 2 by 75% or more, relative to the activity of said at least 12 of the kinases listed in Table 2 under equivalent conditions in the absence of said single agent, in an in vitro kinase assay in which said single agent is present at a concentration of about 3μM. In another specific embodiment, said single agent inhibits at least 21 of the kinases listed in Table 2 by 50% or more, relative to the activity of said at least 21 of the kinases listed in Table 2 under equivalent conditions in the absence of said single agent, in an in vitro kinase assay in which said single agent is present at a concentration of about 3μM.

In another embodiment, the invention provides a method of treating an individual suffering from a condition, comprising administering to said individual a single agent in a therapeutically-effective amount, wherein said single agent detectably inhibits the activity of a plurality of kinases in vivo. In a specific embodiment, said plurality of kinases comprises cSRC, Yes, Fyn, Lck, Fes, Lyn, Syk, Rsk, CDK1, CDK2, CDK3, CDK5, CDK6, CDK7, CHKl, CHK2, JNK1, MAPK1, MAPK2, MAPKAP-K5, MEK1, ROCKII, PRK2, PRAK, p70S6 or Aurora-A; Yes, BMX, Syk, Eph, FGFR, RYK, MUSK, JAK 1 or EGFR; CDK, JNK, ERK, CDKL, ICK, CLK or DYRK; Src, Yes, Fyn, Lyn, Lck, Blk, Hck, Fgr, DYRK, and Yrk; MAPK, MAPK3, ERK2, MAPK7, JNK1, MAPK10, JNK3 alpha or MAPK14; CHK1, CHK2, RSK1, RSK2, RSK3, Aurora-A, Aurora-B, ROCK1, ROCKII or p70S6K; pyruvate dehydrogenase kinase isoenzyme 4 (PDK4), pyruvate dehydrogenase kinase isoenzyme 3 (PDK3), branched chain alpha-ketoacid dehydrogenase kinase (BCKDK) or pyruvate dehydrogenase kinase isoenzyme 1 (PDK1); or CDK1, CDK2, cSrc, Yes, Rsk1 or MEK1. In another specific embodiment, said single agent inhibits the activity of at least one of said plurality of kinases is inhibited detectably, or is inhibited by at least 20%, 25%, 30%, 35%, 40%, 45%, 50%, 55%, 60%, 65%, 70%, 75%, 80%, 85%, 90% or at least 95% by said single agent, relative to the activity of said at least two of said plurality of said kinases under equivalent conditions in the absence of said single agent. In another specific embodiment, said condition is one or more of insulin resistance, diabetes or obesity; inflammation; inappropriate or disease-related angiogenesis; cardiovascular disease; cancer, hyperproliferative disease or related congenital diseases; or infection by a microorganism. In a more specific embodiment, said plurality of kinases comprises CDK1, CHK2, PRK2 and ROCK-II, and said single agent inhibits said CDK1, CHK2, PRK2 and ROCK-II by at least 90%, relative to the activity of said CDK1, CHK2, PRK2 and ROCK-II under equivalent conditions in the absence of said single agent, in an in vitro kinase assay wherein said single agent is present at a concentration of about 3μM. In another specific embodiment, said single agent inhibits at least 7 of the kinases listed in Table 2 by 90% or more, relative to the activity of said at least 7 of the kinases listed in Table 2 under equivalent conditions in the absence of said single agent, in an in vitro kinase assay in which said single agent is present at a concentration of about 3μM. In another specific embodiment, said single agent inhibits at least 12 of the kinases listed in Table 2 by 75% or more, relative to the activity of said at least 12 of the kinases listed in Table 2 under equivalent conditions in the absence of said single agent, in an in vitro kinase assay in which said single agent is present at a concentration of about 3μM. In another specific embodiment, said single agent inhibits at least 21 of the kinases listed in Table 2 by 50% or more, relative to the activity of said at least 21 of the kinases listed in Table 2 under equivalent conditions in the absence of said single agent, in an in vitro kinase assay in which said single agent is present at a concentration of about 3μM.

In one embodiment, the invention provides a method of treating an individual having one or more disease conditions comprising administering an effective dose of the compound CCOO1, wherein an effective dose causes a detectable modulation of the activity of one or more kinases, and wherein said one or more kinases are associated with said one or more disease conditions. In a specific embodiment, said kinase is CDK1/cyclinB, CDK2/cyclinA, cSRC, Yes, MEK or Rsk1. In another embodiment, the invention provides a method of treating an individual having one or more disease conditions comprising administering an effective dose of the compound CC004, wherein an effective dose causes a detectable modulation of the activity of one or more kinases, and wherein said one or more kinases are associated with said one or more disease conditions. In a specific embodiment, said kinase is CDK1/cyclinB, CDK2/cyclinA, cSrc Yes, MEK or Rsk1. In another specific embodiment, said treating comprises administering both CC001 and CC004 to said individual.

### 5.4.2 Compounds

Currently, four compounds have been identified that act a single agents, designated CC001, CC002, CC004, and CC005 (see Example 1). These four compounds have been shown to modulate, and, particularly, to inhibit the activity of several kinases, including Src family kinases and CDK kinases.

As described below, either of the above single agents may be administered to an individual in combination with one or more therapies associated with the particular disease condition to be treated. Thus, the invention also provides a method of treating an individual having one or more disease conditions comprising administering an effective dose of the compound CC004 or CC001 in combination with a second compound, wherein an effective dose causes a detectable modulation of the activity of one or more kinases, wherein said one or more kinases are associated with said one or more disease conditions, and wherein said second compound is a compound other than a single agent.

Of course, either of the above two compounds may be combined with any other single agent, either in a treatment regimen comprising single agents only, or in a regimen comprising other adjuvant agents, as well.

Examples of second, adjuvant agents useful in the treatment of certain disease conditions are listed below.

The invention provides for the use of preferred single agents for the targeting of multiple kinases, and for the treatment of diseases, disorders, and conditions treatable by targeting multiple kinases. In one embodiment, preferred single agents for the targeting of multiple kinases are Indazole Compounds having the Formula (Ia): 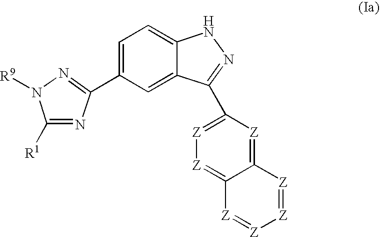
and isomers, prodrugs and pharmaceutically acceptable salts, solvates and hydrates thereof, wherein,
R¹ is —H, -halo, —C₁-C₆ alkyl, —C₁-C₆ alkenyl, —C₁-C₆ alkynyl, —OR³, —N(R⁴)₂, —CN, —NO₂, —C(O)R⁵, —OC(O)R⁵, —NHC(O)R⁵, —SO₂R⁶, -aryl, -heterocycle, -heteroaryl, -cycloalkyl, —(C₁-C₆ alkylene)-R² or —O—(C₁-C₆ alkylene)-R²;
R² is —H, -halo, —C₁-C₆ alkyl, —C₁-C₆ alkenyl, —C₁-C₆ alkynyl, —OR³, —N(R⁴)₂, —CN, —NO₂, —C(O)R⁵, —OC(O)R⁵, —NHC(O)R⁵, —SO₂R⁶, -aryl, -heterocycle, -heteroaryl, or -cycloalkyl;
R³ is independently —H, —C₁-C₆ alkyl, —C₁-C₆ alkenyl, —C₁-C₆ alkynyl, —C₁-C₆ haloalkyl, -cycloalkyl, -aryl, or -heterocycle;
each occurrence of R⁴ is independently —H, —C₁-C₆ alkyl, —C₁-C₆ alkenyl, —C₁-C₆ alkynyl, -cycloalkyl, -aryl, -heterocycle, or —(C₁-C₆ alkylene)-OR³;
R⁵ is —H, —C₁-C₆ alkyl, —C₁-C₆ alkenyl, —C₁-C₆ alkynyl, -cycloalkyl, -aryl, -heterocycle, —OR³, —N(R⁴)₂,
R⁶ is —H, —C₁-C₆ alkyl, —C₁-C₆ alkenyl, —C₁-C₆ alkynyl, —N(R⁴)₂, -cycloalkyl, -aryl, or -heterocycle;
each occurrence of Z is —C(R⁷)— or —N—, wherein up to 3 occurrences of Z can be —N—;
R⁷is —H, -halo, —C₁-C₆ alkyl, —C₁-C₆ haloalkyl, —O—(C₁-C₆ haloalkyl), —C₁-C₆ alkenyl, —C₁-C₆ alkynyl, —OR³, —N(R⁴)₂, —CN, —NO₂, —C(O)R⁵, —OC(O)R⁵, —NHC(O)R⁵, —SO₂R⁶, -aryl, -heterocycle, -cycloalkyl, —C(O)NH—(C₁-C₆ alkylene )ₙ-cycloalkyl, —C(O)NH—(C₁-C₆ alkylene )ₙ-aryl, —C(O)NH—(C₁-C₆ alkylene)ₙ-heterocycle, —(C₁-C₆ alkylene)-cycloalkyl, —(C₁-C₆ alkylene)-aryl, —(C₁-C₆ alkylene)-heterocycle, —O—(C₁-C₆ alkylene)-C(O)R⁵, —O—(C₁-C₆ alkylene)-N(R⁴)₂, —O—(C₁-C₆ alkylene)-cycloalkyl, —O—(C₁-C₆ alkylene)-aryl, or —O—(C₁-C₆ alkylene)-heterocycle, wherein R⁷is attached to the bicyclic ring system via a carbon atom and n is 0 or 1; and
R⁹ is —H, —C₁-C₆ alkyl, or cycloalkyl.

In one embodiment, each occurrence of -Z is —C(R⁷)—.

In another embodiment, R¹ is —H.

In still another embodiment, R¹ is —C₁-C₆ alkyl.

In another embodiment, R¹ is —(C₁-C₆ alkylene)-heterocycle.

In yet another embodiment, R¹ is —CH₂-heterocycle.

In a further embodiment, R¹ is —O—(C₁-C₆ alkylene)-heterocycle.

In another embodiment, R¹ is —O—CH₂-heterocycle.

In another embodiment, R¹ is —(C₁-C₆ alkylene)-N(R⁴)₂, wherein each occurrence of R⁴ is independently —H or C₁-C₆ alkyl.

In another embodiment, R¹ is —(C₁-C₆ alkylene)-NH(C₁-C₆ alkyl).

In another embodiment, R¹ is —(C₁-C₆ alkylene)-N(R⁴)₂, wherein both R⁴ groups are —(C₁-C₆ alkylene)-(O—C₁-C₆ alkyl).

In a embodiment, R¹ is —CH₂—N(R⁴)₂, wherein each occurrence of R⁴ is independently —H or C₁-C₆ alkyl.

In one embodiment, R⁷is —H.

In one embodiment, R⁷is -halo.

In one embodiment, R⁷ is —O—(C₁-C₆ alkyl).

In another embodiment, R⁷ is —O—(C₁-C₆ alkylene)-heterocycle.

In still another embodiment, R⁷ is —C(O)—(C₁-C₆ alkyl).

In another embodiment, R⁷ is —O—(C₁-C₆ haloalkyl).

In one embodiment, R⁸ is —H.

In another embodiment, R⁹is —H.

In still another embodiment, R⁸ is —H and R⁹ is —H.

In a preferred embodiment, R¹ is —(C₁-C₆ alkylene)-heterocycle and R⁷ is —O—(C₁-C₆ alkyl).

In a further preferred embodiment, R₁ is —(C₁-C₆ alkylene)-heterocycle and R⁷ is —O—(C₁-C₆ alkyl), R⁸ is —H and R⁹ is —H.

In another preferred embodiment, R¹ is —(C₁-C₆ alkylene)-N(R⁴)₂, R⁷ is —O—(C₁-C₆ alkyl), R⁸ is —H and R⁹ is —H.

Illustrative Indazole Compounds of Formula (Ia) include the following:

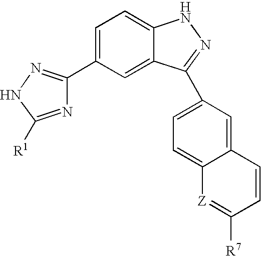

| Com- | | | |
|---|---|---|---|
| pound | R¹ | R⁷ | Z |
| 1 | —H | —OCH₃ | —CH— |
| 2 | —H | —OCH₂CH₃ | —CH— |
| 3 | —H | —O-n-butyl | —CH— |
| 4 | —H | —H | —CH— |
| 5 | —Me | —OCH₃ | —CH— |
| 6 | -isobutyl | —OCH3 | —CH— |
| 7 | -isobutyl | —H | —CH— |
| 8 | —CH₂—pyrrolidin-1-yl | —H | —CH— |
| 9 | —CH₂—pyrrolidin-1-yl | —OCH₂CH₃ | —CH— |
| 10 | —CH₂—pyrrolidin-1-yl | —OH | —CH— |
| 11 | —CH₂—pyrrolidin-1-yl | —C(O)OCH₂CH₃ | —CH— |
| 12 | —CH₂—pyrrolidin-1-yl | —OCHF₂ | —CH— |
| 13 | —CH₂—pyrrolidin-1-yl | —F | —CH— |
| 14 | —CH₂—pyrrolidin-1-yl | —C(O)NHCR₂CH₃ | —CH— |
| 15 | —CH₂—pyrrolidin-1-yl | —O(CH₂)₂—CH(CH₃)₂ | —CH— |
| 16 | —CH₂—pyrrolidin-1-yl | —OH | —CH— |
| 17 | —CH₂—pyrrolidin-1-yl | —OCH₃ | —CH— |
| 18 | —CH₂—pyrrolidin-1-yl | —C(O)NH₂ | —CH— |
| 19 | —CH₂—piperidin-1-yl | —OCH₃ | —CH— |
| 20 | —CH₂—morpholin-1-yl | —OCH₃ | —CH— |
| 21 | —OCH₂-(2-methyl- | —OCH₃ | —CH— |
| 22 | -cyclopentyl | —OCH₃ | —CH— |
| 23 | —(CH₂)₂-piperidin-1-yl | —OCH₃ | —CH— |
| 24 | —CH₂—(1-methyl- piperidin-4-yl) | —OCH₃ | —CH— |
| 25 | —CH₂OH | —OCH₃ | —CH— |
| 26 | —CH(CH₃)—(pyrrolidin- 1-yl) | —OCH₃ | —CH— |
| 27 | —CH₂—(2-methyl- piperidin-1-yl) | —OCH₃ | —CH— |
| 28 | —CH₂—(2,6-dimethyl- piperdin-1-yl | —OCH₃ | —CH— |
| 29 | —CH₂—(azepan-1-yl) | —OCH₃ | —CH— |
| 30 | —CH₂—(piperazin-1-yl) | —OCH₃ | —CH— |
| 31 | —CH₂-(1-acetyl- piperazin-4-yl) | —OCH₃ | —CH— |
| 32 | —CH₂NH₂ | —OCH₃ | —CH— |
| 33 | —CH₂N(CH₃)₂ | —OCH₃ | —CH— |
| 34 | —(CH₂)₂NH₂ | —OCH₃ | —CH— |
| 35 | —CH₂NH—(t-butyl) | —OCH₃ | —CH— |
| 36 | CH₂N(CH₂CH₂OCH₃)₂ | —OCH₃ | —CH— |
| 37 | —CH_{2-piperidin-1-yl} | —OCH₃ | —N— |
| 38 | —CH_{2-morpholin-1-yl} | —H— | —N— |
| 39 | —CH_{2-morpholin-1-yl} | —OCH₃ | —N— |
| 40 | —CH_{2-morpholin-1-yl} | —N(CH₃)₂ | —N— |
| 41 | —CH₂—morpholin-1-yl | —N(H)(CH₃) | —N— |
| 42 | —CH₂—pyrrolidin-1-yl | —OCH₃ | —N— |

and isomers, prodrugs and pharmaceutically acceptable salts, solvates and hydrates thereof.

The present invention also provides for the use of compositions comprising a therapeutically effective amount of a Indazole Compound of Formula (Ia) and a pharmaceutically acceptable vehicle in the targeting of multiple kinases, and the treatment of diseases, disorders or conditions treatable by targeting multiple kinases.

In a particularly preferred embodiment, the single agent is compound 20, above, herein designated “CC001”. See Examples 1 and 85.

In another embodiment, the invention provides Indazole Compounds having the Formula (Ib): 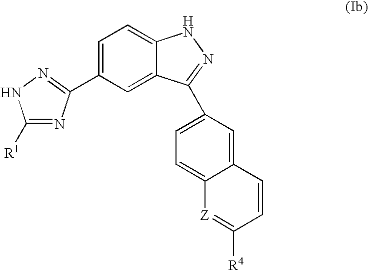
and isomers, prodrugs and pharmaceutically acceptable salts, solvates and hydrates thereof, wherein
R¹ is —H, —C₁-C₆ alkyl, —(C₁-C₆ alkylene)-R² or —O—(C₁-C₆ alkylene)-R²;
R² is —C₁-C₆ alkyl, —C₁-C₆ alkoxy, —OH, —N(R³)₂, -aryl, -heteroaryl, -heterocycle, or -cycloalkyl;
each occurrence of R³ is independently —H, —C₁-C₆ alkyl, or —C₁-C₆ alkylene-C₁-C₆ alkoxy);
R⁴ is —N(R⁵)₂, —O—C₁-C₆ alkyl, —C(O)NH—(C₁-C₆ alkylene)m-heterocycle, —C(O)-heterocycle, —C(O)NH—(C₁-C₆ alkylene)ₘ-heteroaryl, —C(O)-heteroaryl, —(C₁-C₆ alkylene)-cycloalkyl, —O—(C₁-C₆ alkylene)-N(R⁵)₂, —O—(C₁-C₆ alkylene)ₘ-heterocycle, —O—(C₁-C₆ alkylene)ₘ-heteroaryl, —O—(C₁-C₆ alkylene)ₘ-cycloalkyl or —O—(C₁-C₆ alkylene)ₘ-C(O)R⁵;
Z is —CH— or —N—;
each occurrence of R⁵ is independently —H or —C₁-C₆ alkyl; and
m is 0 or 1.

In one embodiment, -Z is —CH—.

In another embodiment, R¹ is —H.

In still another embodiment, R¹ is —C₁-C₆ alkyl.

In another embodiment, R¹ is —(C₁-C₆ alkylene)-heterocycle.

In yet another embodiment, R¹ is —CH₂-heterocycle.

In a further embodiment, R⁴ is —N(R⁵)₂.

In a further embodiment, R⁴is —O—(C₁-C₆ alkylene)-heterocycle.

In a further embodiment, R⁴is —O—C₁-C₆ alkyl, preferable —OCH₃.

In another embodiment, R⁴ is —O—CH₂-heterocycle.

In another embodiment, R⁴ is —O—(CH₂)₂-heterocycle.

In another embodiment, R⁴ is —O—(CH₂)₃-heterocycle.

In another embodiment, R⁴ is —O—(C₁-C₆ alkylene)-heterocycle.

In a preferred embodiment, R¹ is —H and R⁴ is —O—(C₁-C₆ alkylene)-heterocycle.

In another preferred embodiment, R¹ is —CH₂-heterocycle and R⁴ is —O—(C₁-C₆ alkylene)-heterocycle.

In still another preferred embodiment, R¹ is —C₁-C₆ alkyl and R⁴ is —O—(C₁-C₆ alkylene)-heterocycle.

In still another preferred embodiment, Z is —CH—, R¹ is —(C₁-C6alkylene)-R², R² is —N(R³)₂ and R⁴ is —OCH₃.

In still another preferred embodiment, Z is —CH—, R is —C₁-C₆ alkyl and R⁴ is —O—(C₂ alkylene)-N(R⁵)₂.

Illustrative Indazole Compounds of Formula (Ib) include the following:

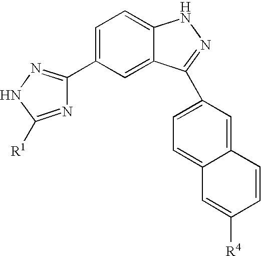

| Compound | R¹ | R⁴ |
|---|---|---|
| 43 | —H | —O(CH₂)₂—(pyrrolidin-1-yl) |
| 44 | —H | —O(CH₂)₂—(piperidin-1-yl) |
| 45 | —Me | —O(CH₂)₂—(azepan-1-yl) |
| 46 | —Me | —O(CH₂)₂—(pyrrolidin-1-yl) |
| 47 | —Me | —O(CH₂)₂—(2,6-dimethyl-piperidin-1-yl) |
| 48 | —CN | —OH |
| 49 | -isopropyl | —O(CH₂)₂—(2(S),6(R)-dimethyl- piperidin-1-yl) |
| 50 | -isobutyl | —O(CH₂)₂—(piperidin-1-yl) |
| 51 | -isobutyl | —O(CH₂)₃—(piperidin-1-yl) |
| 52 | -isobutyl | —O(CH₂)₂—(pyrrolidin-1-yl) |
| 53 | -isobutyl | —O(CH₂)₂—(azepan-1-yl) |
| 54 | -isobutyl | —O(CH₂)₂—(2,6-dimethyl-piperidin-1-yl) |
| 55 | -isobutyl | —O(CH₂)₂—(1-methyl-pyrrolidin-2(R)-yl) |
| 56 | -isobutyl | —O(CH₂)₂—(2-methyl-piperidin-1-yl) |
| 57 | -isobutyl | —O—CH₂—(pyridin-2-yl) |
| 58 | -isobutyl | —O(CH₂)₂—(2(S),6(R)-dimethyl- piperidin-1-yl) |
| 59 | -isobutyl | —O(CH₂)₃—(2(S),6(R)-dimethyl- piperidin-1-yl) |
| 60 | -isobutyl | —OCH₂—(1,4-dimethyl-piperazin-2-yl) |
| 61 | -isobutyl | —O(CH₂)₂—(2,2,6,6-tetramethyl- piperidin-1-yl) |
| 62 | -isobutyl | —O(CH₂)₂—(2-methyl-1-piperidyl) |
| 63 | -isobutyl | —O(CH₂)₂—(2(S)-methyl-1-piperidyl) |
| 64 | -isobutyl | —O(CH₂)₂—(2(R)-methyl-1-piperidyl) |
| 65 | -isobutyl | —O(CH₂)2-(2(S)-methyl-pyrrolidin-1-yl) |
| 66 | -isobutyl | —O(CH₂)2-(2(R)-methy1-pyrrolidin-1-yl) |
| 67 | -isobutyl | —OCH₂—(1-methyl-pyrrolidin-2(S)-yl) |
| 68 | -isobutyl | —OCH₂—(1-methyl-pyrrolidin-2(R)-yl) |
| 69 | -isobutyl | —OCH₂—(1-ethyl-pyrrolidin-2(R)-yl) |
| 70 | -isobutyl | —OCH₂—(1-ethyl-pyrrolidin-2(S)-yl) |
| 71 | -isobutyl | —OCH₂—(2-methyl-pyrrolidin-1-yl-2- one) |
| 72 | -isobutyl | —OCH₂—(3-methyl-3H-imidazol-4-yl) |
| 73 | -isobutyl | —O(CH₂)₂N(CH₃)₂ |
| 74 | -isobutyl | —O(CH₂)₂N(isopropyl)₂ |
| 75 | -isobutyl | —O(CH₂)₂—(2(R),6(S)-dimethyl- piperidin-1-yl) |
| 76 | -isobutyl | —O(CH₂)₃—(2(R),6(S)-dimethyl- piperidin-1-yl) |
| 77 | -isobutyl | —O(CH₂)₂—(2,5-dimethyl-pyrrolidin-1- yl) |
| 78 | -isobutyl | —O(CH₂)₂—(2(R),5(S)dimethyl- pyrrolidin-1-yl) |
| 79 | -isobutyl | —O(CH₂)₂—(2(S),5(R)-dimethyl- pyrrolidin-1-yl) |
| 80 | -isobutyl | —O(CH₂)—C(O)—(2(S),6(R)-dimethyl- piperidin-1-yl) |
| 81 | -isobutyl | —O(CH₂)—C(O)—(2(R),6(S)-dimethyl- piperidin-1-yl) |
| 82 | -t-butyl | —O(CH₂)₂—(pyrrolidin-1-yl) |
| 83 | -t-butyl | —O(CH₂)₂—(azepan-1-4) |
| 84 | -t-butyl | —O(CH₂)₂—(piperidin-1-yl) |
| 85 | -t-butyl | —O(CH₂)₂—(2(S),6(R)-dimethyl- piperidin-1-yl) |
| 86 | -t-butyl | —OCH₂—(1-methyl-pyrrolidin-2(S)-yl) |
| 87 | -t-butyl | —OCH₂—(1-methyl-pyrrolidin-2(R)-yl) |
| 88 | -t-butyl | —OCH₂—(1,4-dimethyl-piperazin-2-yl) |
| 89 | -t-butyl | —O(CH₂)₂-imidazol-1-yl |
| 90 | -neopentyl | —F |
| 91 | -neopentyl | —O-(pyridin-2-yl) |
| 92 | -neopentyl | —OCH₂-(pyridin-2-yl) |
| 93 | -neopentyl | —O(CH₂)₂—-(pyridin-3-yl) |
| 94 | -neopentyl | —OCH₂-(1,4-dimethyl-piperazin-2-yl) |
| 96 | -neopentyl | —C(O)NH(CH₂)₂-pyrrolidin-1-yl) |
| 97 | -neopentyl | —OCH₂-(1-methyl-piperidin-2-yl) |
| 98 | -neopentyl | —O(CH₂)₂—(2(S),6(R)-dimethyl- piperidin-1-yl) |
| 99 | -neopentyl | —O(CH₂)₂—(2(R),6(S)-dimethyl- piperidin-1-yl) |
| 100 | -neopentyl | —O(CH₂)—C(O)—(2(R),6(S)-dimethyl- piperidin-1-yl) |
| 101 | -neopentyl | —O(CH₂)—C(O)—(2(S),6(R)-dimethyl- piperidin-1-yl) |
| 102 | -neopentyl | —OCH₂—(1-methyl-pyrrolidin-2(S)-yl) |
| 103 | -neopentyl | —OCH₂—(1 -methyl-pyrrolidin-2(R)-yl) |
| 104 | -neopentyl | —O(CH₂)₂—(pyrrolidin-1-yl) |
| 105 | -neopentyl | —O(CH₂)₂—(2,5-dimethyl-pyrrolidin-1- yl) |
| 106 | -neopentyl | —O(CH₂)₂—(2(R),5(S)-dimethyl- pyrrolidin-1-yl) |
| 107 | -neopentyl | —O(CH₂)₂—(2(S),5(R)-dimethyl- pyrrolidin-1-yl) |
| 108 | -neopentyl | —O—(CH₂)₂—N(ethyl)(isopropyl) |
| 109 | -neopentyl | —O(CH₂)₃—(2(S),6(R)-dimethyl- piperidin-1-yl) |
| 110 | -neopentyl | —O(CH₂)₃—(2(S),6(R)-dimethyl- piperidin-1-yl) |
| 111 | -neopentyl | —OCH₂—(1,4-dimethyl-piperazin-2-yl) |
| 112 | -neopentyl | —O(CH₂)₂—(pyrrolidin-1-yl-2-one) |
| 113 | -neopentyl | —OCH₂CH(isopropyl)(pyrrolidin-1-yl) |
| 114 | -neopentyl | —OCH₂—(1-methyl-piperazin-2-yl) |
| 115 | -neopentyl | —O(CH₂)₂—(imidazol-1-yl) |
| 116 | -neopentyl | —O(CH₂)₂—(2(S)-methyl-piperidin-1-yl) |
| 117 | -neopentyl | —OCH₂—(1-ethyl-pyrrolidin-2(S)-yl) |
| 118 | -neopentyl | —OCH₂—(1-ethyl-pyrrolidin-2(R)-yl) |
| 119 | -neopentyl | —O(CH₂)₂—(2,2,6,6-tetramethyl- piperidin-1-yl) |
| 120 | -neopentyl | —OCH₂—(1-methyl-pyrrolidin-2(R)-yl) |
| 121 | -neopentyl | —O(CH₂)₂N(isopropyl)₂ |
| 122 | -neopentyl | —O—(CH₂)₂—3-methyl-imidazolidin-2-one |
| 123 | -isopropyl | —O(CH₂)₂—(2(S),6(R)-dimethyl- piperidin-1-yl |
| 124 | —CH₂—pyrrolidin-1-yl | —Cl |
| 125 | —CH₂—pyrrolidin-1-yl | —O(CH₂)₂—(piperidin-1-yl) |
| 126 | —CH₂—pyrrolidin-1-yl | —O(CH₂)₂—(pyrrolidin-1-yl) |
| 127 | —CH₂—pyrrolidin-1-yl | —O(CH₂)₂—(azepan-1-yl) |
| 128 | —CH₂—pyrrolidin-1-yl | —O(CH₂)₂—cyclopentyl piperidin-1-yl |
| 129 | | |
| 130 | —CH₂—pyrrolidin-1-yl | —O(CH₂)₃—(2(S)-methyl-piperidin-1-yl) |
| 131 | —CH₂—pyrrolidin-1-yl | —OCH₂—(1-methyl-pyrrolidin-2(S)-yl) |
| 132 | —CH₂—pyrrolidin-1-yl | —OCH₂—(1-methyl-pyrrolidin-2(R)-yl) |
| 133 | —CH₂—pyrrolidin-1-yl | —OCH₂—(pyrrolidin-1-yl-2-one) |
| 134 | —CH₂—pyrrolidin-1-yl | —O(CH₂)₂—pyrrolidin-1-yl-2-one |
| 135 | —CH₂—pyrrolidin-1-yl | —OCH₂—(2-methyl-pyrrolidin-1-yl-2- one) |
| 136 | —CH₂—pyrrolidin-1-yl | —C(O)—(pyrrolidin-1-yl) |
| 137 | —CH₂—pyrrolidin-1-yl | —OCH₂—(pyridin-2-yl) |
| 138 | —CH₂—pyrrolidin-1-yl | —O(CH₂)₂—(pyridin-2-yl) |
| 139 | —CH₂—pyrrolidin-1-yl | —O(CH₂)₂—(pyridin-3-yl) |
| 140 | —CH₂—pyrrolidin-1-yl | —O(CH₂)₂N(CH₃)₂ |
| 141 | —CH₂—pyrrolidin-1-yl | —O(CH₂)₂N(CH₃)₂ |
| 142 | —CH₂—pyrrolidin-1-yl | —O(CH₂)₂—(2(S)-methyl-1-piperidyl) |
| 143 | —CH₂—pyrrolidin-1-yl | —O(CH₂)₂—(2(R)-methyl-1-piperidyl) |
| 144 | —CH₂—morpholin-1-yl | —OCHF₂ |
| 145 | —CH₂—morpholin-1-yl | —O(CH₂)₂—(pyrrolidin-1-yl) |
| 146 | —CH₂—morpholin-1-yl | —O(CH₂)₂—(piperidin-1-yl) |
| 147 | —CH₂—morpholin-1-yl | —OCH₂—(pyridin-2-yl) |
| 148 | —CH₂—morpholin-1-yl | —O(CH₂)₂—(pyridin-2-yl) |
| 149 | —CH₂—cyclopentyl | —O(CH₂)₂—(2(S),6(R)-dimethyl piperidin-1-yl |
| 150 | —CH₂—cyclopropyl | —O(CH₂)₂—piperidin-1-yl) |
| 151 | —CH₂—cyclopropyl | —O(CH₂)₂—pyrrolidin-1-yl) |
| 152 | —CH₂—cyclopropyl | —O(CH₂)₂—(azepan-1-yl) |
| 153 | —CH₂—cyclopropyl | —O(CH₂)₂—(2(S),6(R)-dimethyl- piperidin-1-yl) |
| 154 | —CH₂—O-t-butyl | —O(CH₂)₂—(2,S),6(R)-dimethyl- piperidin-1-yl) |
| 155 | —CH₂—O-t-butyl | —O(CH₂)₂—pyrrolidin-1-yl-2-one |
| 156 | —CH₂—NH(t-butyl) | —OCH₃ |
| 157 | —CH₂—N((CH₂)₂—O—CH₃)₂ | —OCH₃ |
| 158 | 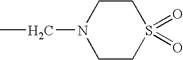 | —OCH₃ |

and isomers, prodrugs and pharmaceutically acceptable salts, solvates and hydrates thereof.

The present invention also provides for the use of compositions comprising a therapeutically effective amount of a Indazole Compound of Formula (Ib) and a pharmaceutically acceptable vehicle in the targeting of multiple kinases, and the treatment of diseases, disorders or conditions treatable by targeting multiple kinases.

In a particularly preferred embodiment, the single agent is compound 52, above, herein designated “CC002”. In another particularly preferred embodiment, the single agent is compound 92, above, herein designated “CC004”. In a particularly preferred embodiment, the single agent is compound 117, above, herein designated “CC005”. See Examples 1 and 86-88.

In another embodiment, the invention provides for the use of a single agent to target (e.g.,inhibit) a plurality of kinases, and the treatment of a disease, condition or disorder treatable by targeting (e.g., inhibiting) said plurality of protein kinases, wherein said single agent is not 5-(5-cyclopentyl-1H-[1,2,4]triazol-3-yl)-3-(4-fluoro-phenyl)-1H-indazole: 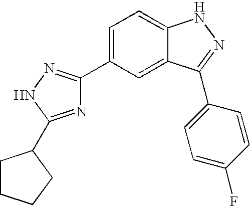
or 3-(5-(1H-1,2,4-triazol-3-yl)(1H-indazol-3-yl))phenyl-N-cyclopentylcarboxamide:

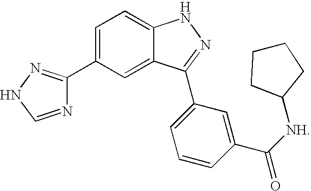

In another embodiment, the methods of the invention encompass the targeting of a plurality of kinases using a single agent that is a small (<1000 daltons) organic molecule that is not an indazole-containing compound. In another embodiment, the methods of the invention encompass the targeting of a plurality of protein kinases using a single agent that is a nucleic acid such as an aptamer that modulates the activity of said plurality of protein kinases. In another embodiment, the methods of the invention encompass the targeting of a plurality of protein kinases using a single agent that is a peptide, wherein the peptide modulates the activity of said multiple kinases. These single agents, and other disclosed herein, may be used in conjunction with one another. For example, a single agent that is a indazole-containing compound may be used in conjunction with a single agent that is an indazole-containing compound, a polynucleotide or a peptide, or any combination thereof; a single agent that is a peptide may be used in conjunction with a single agent that is an indazole-containing compound, a non-indazole-containing compound or a polynucleotide; etc. In a specific embodiment, the methods of the invention encompass the targeting of a plurality of protein kinases using a peptide, a polynucleotide or a non-indazole small molecule, wherein said plurality of protein kinases are CDK kinases, Yes kinases, cSrc kinases, MEK kinases and Rsk kinases. In a more specific embodiment, said plurality of kinases comprise human CDK1, CDK2, Yes, MEK1, cSRC and Rsk1.

### 5.4.3 Adjunctive Therapy

According to the invention, therapy by administration of one or more single agents may be combined with the administration of one or more therapies such as, but not limited to, chemotherapies, radiation therapies, hormonal therapies, and/or biological therapies/immunotherapies.

### 5.4.3.1 Cancer Adjunctive Therapy

In a specific embodiment, the methods of the invention encompass the administration, in combination with one or more single agents, of one or more angiogenesis inhibitors such as but not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); Fibronectin fragment; Gro-beta; Halofuginone; heparinases; heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C1; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; ZD 6474; famesyl transferase inhibitors (FTI); and bisphosphonates.

Additional examples of anti-cancer agents that can be used in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefuir; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-i receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomvcin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. These two agents are particularly useful when used in methods employing thalidomide and a topoisomerase inhibitor.

In more particular embodiments, the present invention also comprises the administration of one or more single agents in combination with the administration of one or more therapies such as, but not limited to anti-cancer agents such as those disclosed in Table

TABLE 1
Anti-cancer agents

| Therapeutic Agent | Dose/Administration/Formulation | | |
|---|---|---|---|
| Doxorubicin | Intravenous | 60-75 mg/m² on Day 1 | 21 day intervals |
| hydrochloride | | | |
| (Adriamycin RDF ® | | | |
| and | | | |
| Adriamycin PFS ®) | | | |
| epirubicin | Intravenous | 100-120 mg/m² on Day 1 of each | 3-4 week cycles |
| hydrochloride | | cycle or | |
| (Ellence ™) | | divided equally and given on | |
| | | Days 1-8 of the cycle | |
| fluorousacil | Intravenous | How supplied: | |
| | | 5 mL and 10 mL vials (containing | |
| | | 250 and 500 mg flourouracil | |
| | | respectively) | |
| docetaxel | Intravenous | 60-100 mg/m² over 1 hour | Once every 3 weeks |
| (Taxotere ®) | | | |
| paclitaxel | Intravenous | 175 mg/m² over 3 hours | Every 3 weeks for |
| (Taxol ®) | | | 4 courses (administered |
| | | | sequentially to |
| | | | doxorubicin-containing |
| | | | combination chemotherapy) |
| tamoxifen citrate | Oral | 20-40 mg | Daily |
| (Nolvadex ®) | (tablet) | Dosages greater than 20 mg | |
| | | should be given in divided doses | |
| | | (morning and evening) | |
| leucovorin calcium | Intravenous or | How supplied: | Dosage is unclear from text. |
| for injection | intramuscular | 350 mg vial | PDR 3610 |
| | injection | | |
| luprolide acetate | Single | 1 mg (0.2 mL or 20 unit mark) | Once a day |
| (Lupron ®) | subcutaneous | | |
| | injection | | |
| flutamide | Oral (capsule) | 250 mg | 3 times a day at 8 hour |
| (Eulexin ®) | | (capsules contain 125 mg | intervals (total daily dosage |
| | | flutamide each) | 750 mg) |
| nilutamide | Oral | 300 mg or 150 mg | 300 mg once a day for 30 days |
| (Nilandron ®) | (tablet) | (tablets contain 50 or 150 mg | followed by 150 mg once a day |
| | | nilutamide each) | |
| bicalutamide | Oral | 50 mg | Once a day |
| (Casodex ®) | (tablet) | (tablets contain 50 mg | |
| | | bicalutamide each) | |
| progesterone | Injection | USP in sesame oil 50 mg/mL | |
| ketoconazole | Cream | 2% cream applied once or twice | |
| (Nizoral ®) | | daily depending on symptoms | |
| prednisone | Oral | Initial dosage may vary from 5 mg | |
| | (tablet) | to 60 mg per day depending | |
| | | on the specific disease entity | |
| | | being treated. | |
| estramustine | Oral | 14 mg/kg of body weight (i.e. | Daily given in 3 or 4 divided |
| phosphate sodium | (capsule) | one 140 mg capsule for each 10 kg | doses |
| (Emcyt ®) | | or 22 lb of body weight) | |
| etoposide or | Intravenous | 5 mL of 20 mg/mL solution (100 mg) | |
| VP-16 | | | |
| dacarbazine | Intravenous | 2-4.5 mg/kg | Once a day for 10 days. |
| (DTIC-Dome ®) | | | May be repeated at 4 week |
| | | | intervals |
| polifeprosan 20 with | wafer placed in | 8 wafers, each containing 7.7 mg | |
| carmustine implant | resection cavity | of carmustine, for a total of 61.6 mg, | |
| (BCNU) | | if size and shape of resection | |
| (nitrosourea) | | cavity allows | |
| (Gliadel ®) | | | |
| Cisplatin | Injection | [n/a in PDR 861] | |
| | | How supplied: | |
| | | solution of 1 mg/mL in multi-dose | |
| | | vials of 50 mL and 100 mL | |
| mitomycin | Injection | supplied in 5 mg and 20 mg vials | |
| | | (containing 5 mg and 20 mg | |
| | | mitomycin) | |
| gemcitabine HCl | Intravenous | For NSCLC-2 schedules have | 4 week schedule- |
| (Gemzar ®) | | been investigated and the | Days 1, 8 and 15 of each 28-day |
| | | optimum schedule has not been | cycle. Cisplatin intravenously |
| | | determined | at 100 mg/m² on day 1 after |
| | | 4 week schedule- | the infusion of Gemzar. |
| | | administration intravenously at | 3 week schedule- |
| | | 1000 mg/m² over 30 minutes on 3 | Days 1 and 8 of each 21 day |
| | | week schedule- | cycle. Cisplatin at dosage of |
| | | Gemzar administered | 100 mg/m² administered |
| | | intravenously at 1250 mg/m² over | intravenously after |
| | | 30 minutes | administration of Gemzar on |
| | | | day 1. |
| carboplatin | Intravenous | Single agent therapy: | Every 4 weeks |
| (Paraplatin ®) | | 360 mg/m² I.V. on day 1 | |
| | | (infusion lasting 15 minutes or | |
| | | longer) | |
| | | Other dosage calculations: | |
| | | Combination therapy with | |
| | | cyclophosphamide, Dose | |
| | | adjustment recommendations, | |
| | | Formula dosing, etc. | |
| ifosamide | Intravenous | 1.2 g/m² daily | 5 consecutive days |
| (Ifex ®) | | | Repeat every 3 weeks or after |
| | | | recovery from hematologic |
| | | | toxicity |
| topotecan | Intravenous | 1.5 mg/m² by intravenous | 5 consecutive days, starting on |
| hydrochloride | | infusion over 30 minutes daily | day 1 of 21 day course |
| (Hycamtin ®) | | | |

The invention also encompasses administration of one or more single agents in combination with radiation therapy comprising the use of x-rays, gamma rays and other sources of radiation to destroy the cancer cells. In preferred embodiments, the radiation treatment is administered as external beam radiation or teletherapy wherein the radiation is directed from a remote source. In other preferred embodiments, the radiation treatment is administered as internal therapy or brachytherapy wherein a radioactive source is placed inside the body close to cancer cells or a tumor mass.

Cancer therapies and their dosages, routes of administration and recommended usage are known in the art and have been described in such literature as the Physician's Desk Reference (₅₆th ed., 2002).

### 5.4.3.2 Inflammation Adjuvant Therapy

To treat inflammations and inflammation-related conditions, administration of one or more single agents may be combined with one or more compounds having anti-inflammatory activity. In various embodiments, therefore, treatment with a single agent may be combined with treatment with, for example, steroidal anti-inflammatory agents, nonsteriodal anti-inflammatory agents (NSAIDs), Benadryl®, IL-9 antagonists, antihistamines, sympthomimetics, glucocorticoids, corticosteroids, β-adrenergic drugs (epinephrine and isoproterenol), theophylline, anticholinergic drugs (e.g., atropine and ipratropium bromide), leukotriene inhibitors, immunotherapies (e.g., repeated long-term injection of allergen, short course desensitization, venom immunotherapy, an effective amount of one or more anti-IgE antibodies and/or one or more mast cell modulators (e.g., a mast cell protease inhibitor, stem cell factor (c-kit ligand) inhibitor, and c-kit receptor inhibitor).

### 5.4.4 Dosage and Administration

The present invention relates to the administration of one or more compounds, also called single agents, that act to target two or more kinases, or the genes encoding them, simultaneously.

In a preferred embodiment, a single agent is a pharmaceutical composition. Such compositions comprise a prophylactically or therapeutically effective amount of one or more single agents and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions are sterile and in suitable form for administration to a subject, preferably an animal subject, more preferably a mammalian subject, and most preferably a human subject.

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering one or more prophylactic or therapeutic agents, care must be taken to use materials to which the prophylactic or therapeutic agents do not absorb.

In another embodiment, the single agent can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 3 17-327; see generally ibid.).

In yet another embodiment, the single agent can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the invention or fragments thereof. See e.g., U.S. Pat. No. 4,526,938, PCT publication WO 91/05548, PCT publication WO 96/20698.Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety. (0161] In a specific embodiment where the single agent of the invention is one or more nucleic acid molecules encoding one or more prophylactic or therapeutic agents, the nucleic acid can be administered in vivo to promote expression of its encoded prophylactic or therapeutic agents, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

If the single agent is to be administered topically, it can be formulated in the form of, e.g., an ointment, cream, transdermal patch, lotion, gel, shampoo, spray, aerosol, solution, emulsion, or other form well-known to one of skill in the art. See e.g., Remington's Pharmaceutical Sciences and Introduction to Pharmaceutical Dosage Forms, 4^{th} ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well-known in the art.

If the compositions of the invention are to be administered intranasally, the compositions can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

If the compositions of the invention are to be administered orally, the compositions can be formulated orally in the form of, e.g., tablets, capsules, cachets, gelcaps, solutions, suspensions and the like. Tablets or capsules can be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated for slow release, controlled release or sustained release of a prophylactic or therapeutic agent(s).

The compositions of the invention may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions of the invention may also be formulated in rectal compositions such as suppositories or retention enemas, e.g., containing conventional suppository bases such as cocoa butter or other glycerides.

In addition to the formulations described previously, the compositions of the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In particular, the invention provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted, e.g., with water or saline to the appropriate concentration for administration to a subject. Preferably, one or more of the prophylactic or therapeutic agents, or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents, or pharmaceutical compositions of the invention should be stored at between 2 and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents, or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

The single agent may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### 6. EXAMPLES

### 6.1 Example 1

### Identification of Single Agent Kinase Inhibitors

Six compounds (presumptive single agents) were tested against a panel of 50 kinases. The results are presented in Table 2; numbers for each kinase indicate the percent of control kinase activity in the presence of a 3μM concentration of the indicated agent.

TABLE 2
Inhibitory activity (% of control) of six presumptive
single agents against 50 kinases.

| | CC001 | CC004 | CC005 | CC002 | CC006 | CC007 |
|---|---|---|---|---|---|---|
| | 3 μM | 3 μM | 3 μM | 3 μM | 3 μM | 3 μM |
| AMPK(r) | 52 | 54 | 2 | 4 | 74 | 74 |
| Aurora-A(h) | 7 | 7 | 0 | ND | ND | ND |
| Blk(m) | 25 | 43 | 4 | 11 | 60 | 76 |
| CaMKII(r) | 64 | 52 | 4 | 26 | 82 | 68 |
| CaMKIV(h) | 82 | 80 | 22 | 60 | 95 | 86 |
| CDK1/ | 2 | 1 | 1 | 2 | 3 | 26 |
| cyclinB(h) | | | | | | |
| CDK2/ | 4 | 2 | 2 | ND | ND | ND |
| cyclinA(h) | | | | | | |
| CDK3/ | 31 | 39 | 25 | 36 | 15 | 73 |
| cyclinE(h) | | | | | | |
| CDK5/p35(h) | 7 | 10 | 8 | 16 | 12 | 50 |
| CDK6/ | 82 | 80 | 29 | 59 | 63 | 87 |
| cyclinD3(h) | | | | | | |
| CDK7/ | 13 | 19 | 7 | ND | ND | ND |
| cyclinH/MAT1 | | | | | | |
| CHK1(h) | 36 | 64 | 6 | 12 | 70 | 90 |
| CHK2(h) | 2 | 7 | 1 | 1 | 20 | 6 |
| CK2(h) | 45 | 98 | 74 | 41 | 50 | 27 |
| c-RAF(h) | 99 | 85 | 100 | 96 | 99 | 92 |
| CSK(h) | 95 | 123 | 88 | ND | ND | ND |
| cSRC(h) | 2 | 67 | 3 | ND | ND | ND |
| Yes(h) | 2 | 24 | 0 | −3 | 57 | 50 |
| Fyn(h) | 14 | 67 | 4 | 4 | 65 | 66 |
| Lck(h) | 27 | 57 | 6 | 5 | 43 | 56 |
| Fes(h) | 34 | 70 | 8 | 10 | 70 | 41 |
| Lyn(h) | 5 | 62 | 2 | 2 | 23 | 61 |
| Syk(h) | 26 | 47 | 15 | 14 | 46 | 51 |
| Bmx(h) | 74 | 98 | 36 | ND | ND | ND |
| FGFR3(h) | 33 | 77 | 5 | 5 | 33 | 54 |
| GSK3α(h) | 15 | 65 | 35 | 38 | 38 | 18 |
| GSK3β(h) | 12 | 63 | 34 | ND | ND | ND |
| IGF-1R(h) | 92 | 96 | 58 | 55 | 65 | 88 |
| IR(h) | 74 | 89 | 31 | ND | ND | ND |
| MAPK2(h) | 0 | 1 | 2 | 2 | 3 | 3 |
| MAPKAP- | 97 | 108 | 56 | 72 | 96 | 86 |
| K2(h) | | | | | | |
| MEK1(h) | 4 | 11 | 2 | ND | ND | ND |
| MSK1(h) | 62 | 76 | 25 | 45 | 72 | 89 |
| p70S6K(h) | 17 | 40 | 3 | 16 | 41 | 83 |
| PAK2(h) | 68 | 62 | 13 | 21 | 56 | 86 |
| PDGFRα(h) | 72 | 77 | 28 | 71 | 86 | 108 |
| PDGFRβ(h) | 94 | 91 | 20 | ND | ND | ND |
| PDK1(h) | 59 | 70 | 59 | 58 | 43 | 97 |
| PKA(h) | 60 | 91 | 50 | ND | ND | ND |
| PKCε(h) | 82 | 105 | 84 | 58 | 82 | 79 |
| PKCμ(h) | 6 | 34 | 2 | ND | ND | ND |
| PKD2(h) | 14 | 26 | 0 | ND | ND | ND |
| PRAK(h) | 12 | 24 | 10 | 6 | 12 | 0 |
| PRK2(h) | 9 | 3 | 1 | 4 | 24 | 68 |
| ROCK-II(h) | 4 | 5 | 2 | 2 | 13 | 69 |
| Rsk1(h) | 4 | 29 | 2 | 3 | 43 | 51 |
| Rsk2(h) | 6 | 34 | 3 | 5 | 31 | 45 |
| Rsk3(h) | 4 | 43 | 4 | 5 | 39 | 38 |
| SGK(h) | 56 | 33 | 4 | ND | ND | ND |
| ZAP-70(h) | 105 | 68 | 26 | 40 | 77 | 87 |

ND: Not done

Compounds CC001, CC002 and CC004 were the most active against the kinases tested, inhibiting the kinase activity of 16, 29 and 16 kinases listed in Table 2, respectively, by more than 95% in each case compared to a no-compound control. CC004 showed good inhibitory activity against CDKl and CDK2, MEK1 and Rsk1, and somewhat less inhibitory activity against the Src family kinases cSrc and Yes. CC006 (5-(5-Cyclopentyl-1H-[1,2,4]triazol-3-yl)-3-(4-fluoro-phenyl)-1H-indazole) and CC007 (3-(5-(1H-1,2,4-triazol-3-yl)(1H-indazol-3-yl))phenyl-N-cyclopentylcarboxamide) were included as controls.

### 6.2 Example 2

### In Vitro Effects of a Single Agent Capable of Targeting Multiple Kinases or Kinase Pathways

CC001 is a low molecular weight mixed kinase inhibitor (MKI) with potent in vitro inhibitory activity against a variety of kinases. CC001 inhibited the activation of kinases and their downstream targets in HCT-116 colon cancer cells in a concentration-dependent manner. CC001 also showed potent antiproliferative activity against a broad spectrum of cancer cell lines, including: non-small cell lung (NSCLC); colon; pancreatic; head and neck; and breast and ovarian cancers. In each case, IC50 values lay in the nanomolar range. In in vitro combination studies with standard chemotherapeutic agents such as taxol, and novel signal transduction inhibitors, CC001 showed additive and/or synergistic antiproliferative activity.

CC001 shows cancer inhibitory effect in in vivo cancer models. SCID mice bearing colon and lung tumors were treated with CC001 at a concentration of 10 mg/kg to 20mg/kg, administered twice a day. CC001 inhibited tumor growth in a dose-dependent manner as a single agent (T/C ratio: 40-60%).

A similar study was performed using an NCI-H460 NSCLC xenograft model in which CC001 was combined with the maximum tolerated dose of Taxotere (an anti-cancer compound related to taxol). In this experiment, a T/C ratio of 16-28% was observed. No apparent additional toxicity as evidenced by weight loss was observed. Additive in vivo effects were also observed with Camptosar (anti-cancer agent generally used for colorectal cancers), with a T/C ratio approaching 15%.

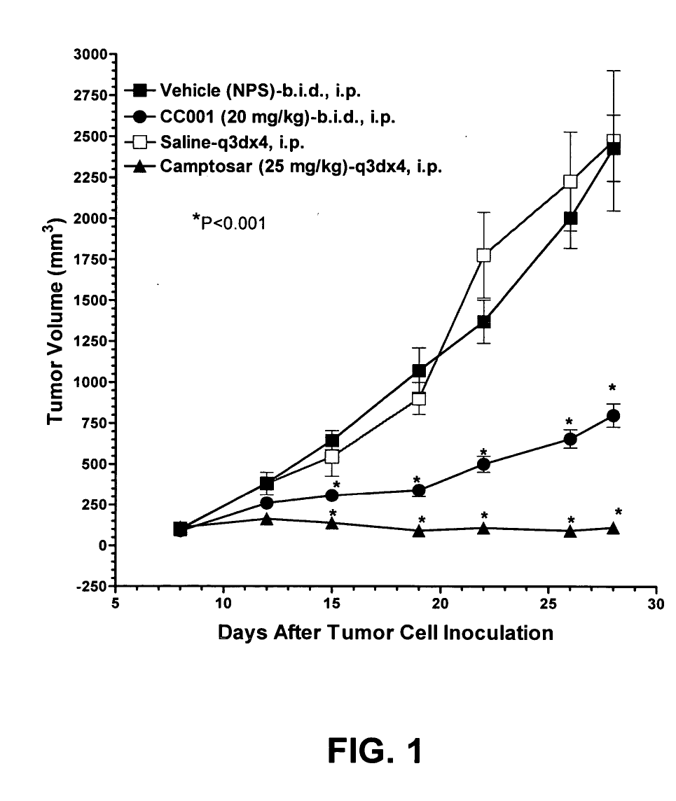

In one experiment, female CB.17 SCID mice were inoculated subcutaneously in the right hind limb with 2×10⁶ HCT-116 cells. After 8 days mice were selected for tumors generally between 75 and 125 mm³ in size and randomly dispersed into groups. Treatment commenced on day 8 and proceeded for the duration of the study. All the compound treatments were given i.p. in a vehicle NPS in a volume of 5 ml/kg. CC001 was administered at 20 mg/kg b.i.d. Camptosar was given as i.p q4d. The tumor volumes were measured twice a week using calipers. Values are mean±SEM. Statistical analysis was performed using ANOVA. As shown in FIG. 1 and in Table 3, treatment with CC001 resulted in a significantly smaller tumor mass over the course of the experiment (P<0.001 compared to vehicle control).

TABLE 3
Treatment of SCID mouse/HCT-116 xenograft model with C001

| | | | | | % Tumor | | |
|---|---|---|---|---|---|---|---|
| | Route of | Dose | | Tumor | Growth | | |
| | Administration | (mg/kg) | Schedule | Volume^{a,b} | Inhibition^{a} | T/C^{a,c} | TGD^{d} |
| CC001 | i.p. | 20 | b.i.d. | 798.3 ± 71.1^{e} | 69.6 | 32 | >12 d |
| Camptosar | i.p. | 25 | q4d | 110.2 ± 8.9^{e} | 95.4 | 4.5 | >12 d |
| Vehicle | i.p. | | b.i.d. | 2430 ± 203 | 0 | | |

^{a}At day 28
^{b}Mean (mm³) ± SEM
^{c}Ratio of mean tumor volume for treated vs. vehicle control
^{d}days to reach tumors to reach 1000 mm³
^{e}P values vs. vehicle control <0.001 (ANOVA)

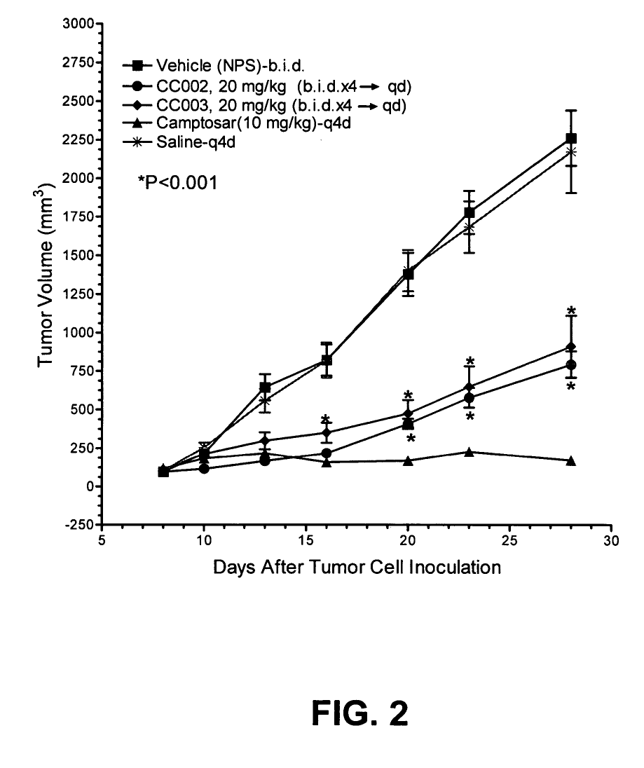

In a related experiment, two compounds, designated CC002 and CC003, were tested. CC003 is a naphthalene-containing indazole. Female CB.17 SCID mice were inoculated subcutaneously in the right hind limb with 2×10⁶ HCT-116 cells. After 8 days mice were selected for tumors generally between 75 and 125 mm³ in size and randomly dispersed into groups. Treatment commenced on day 8 and proceeded for the duration of the study. All the treatments were given i.p. in a vehicle NPS in a volume of 5 ml/kg. CC002 and CC003 administered at 20 mg/kg b.i.d. for first 4 days (day 8-11 and then switched to qd. Camptosar was given at 25 mg/kg q4d. The tumor volumes were measured twice a week using calipers. Values are mean±SEM. Statistical analysis was performed using ANOVA. As shown in FIG. 2 and in Table 4, treatment with either CC002 and CC003 resulted in reduced tumor volumes over the course of the experiment. (P<0.001 when compared with vehicle control.)

TABLE 4
Treatment of SCID mouse/HCT-116 xenograft model with CC002 or CC003

| | | | | | % Tumor | | |
|---|---|---|---|---|---|---|---|
| | Route of | Dose | | Tumor | Growth | | |
| | Administration | (mg/kg) | Schedule | Volume^{a,b} | Inhibition^{a} | T/C^{a,c} | TGD^{d} |
| CC002 | i.p. | 20 | b.i.d. | 792.8 ± 86.5^{e} | 68 | 35 | >12 |
| CC003 | i.p. | 20 | b.i.d. | 910.3 ± 199.5^{e} | 63 | 40 | >12 |
| Vehicle | i.p. | | b.i.d. | 2262.2 ± 180.5 | 0 | 0 | |

^{a}At day 28
^{b}Mean (mm³) ± SEM
^{c}Ratio of mean tumor volume for treated vs. vehicle control
^{d}days to reach tumors to reach 1000 mm³
^{e}P values vs. vehicle control <0.001 (ANOVA)

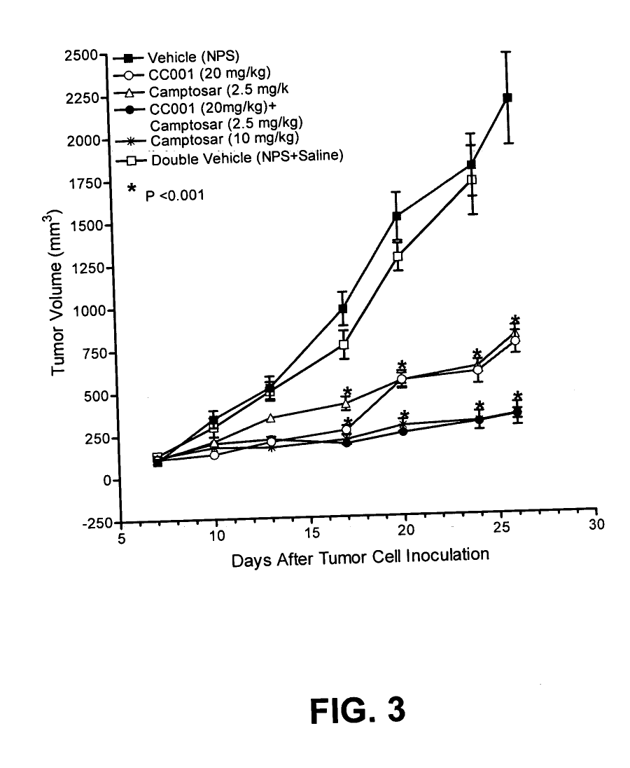

Compound C001 shows increased effect when administered with a second compound. Female CB.17 SCID mice were inoculated subcutaneously in the right hind limb with 2×10⁶ HCT-116 cells. After 8 days mice were selected for tumors generally between 75 and 125 mm³ in size and randomly dispersed into groups. Treatment commenced on day 8 and proceeded for the duration of the study. All the compound treatments were given i.p. in a vehicle NPS in a volume of 5 ml/kg. CC001 was administered at 20 mg/kg b.i.d. Camptosar was given as i.p q4d. The tumor volumes were measured twice a week using calipers. Values are mean±SEM. Statistical analysis was performed using ANOVA. As shown in FIG. 3 and in Table 5, CC001 at 20 mg/kg administered with 2.5 mg/kg Camptosar showed an effect on tumor development equivalent to an administration of Camptosar alone at 10 mg/kg. (P<0.001 when compared with vehicle control.)

TABLE 5
Treatment of SCID mouse/HCT-116 xenograft model with C001, with or without Camptosar

| | | | | | % Tumor | | | |
|---|---|---|---|---|---|---|---|---|
| | Route of | | | Tumor | Growth | | TGD | TGD |
| | Administration | Dose (mg/kg) | Schedule | Volume^{a,b} | Inhibition^{a} | T/C^{a,c} | (500 mm³)^{d} | (1000 mm³)^{d} |
| CC001 | i.p. | 20 | b.i.d. | 752.5 ± 66.6^{e} | 69 | 34 | >7 | >10 |
| Camptosar | i.p. | 2.5 | q4d | 800.5 ± 51.5^{e} | 66 | 37 | >6 | >10 |
| CC001 + | i.p. | 20 (CC001) | b.i.d. (CC001) & | 332.8 ± 28.5^{e} | 90 | 15 | >13 | >10 |
| Camptosar | | & 2.5 | q4d (Camptosar) | | | | | |
| | | (Camptosar) | | | | | | |
| Camptosar | i.p. | 10 | q4d | 335.7 ± 67.8^{e} | 90 | 15 | >13 | >10 |
| Vehicle | i.p. | | b.i.d. | 2179.3 ± 268.7 | | | | |

^{a}At day 28
^{b}Mean (mm³) ± SEM
^{c}Ratio of mean tumor volume for treated vs. vehicle control
^{d}days to reach tumors to reach 500 or 1000 mm³
^{e}P values vs. vehicle control < 0.001 (ANOVA)

### 6.3 Example 3

### In Vivo Assays for Assessing Candidate Single Agent

Single agents may be confirmed or finally identified using a variety of in vivo models. Candidate single agents are typically first identified in in vitro screens, and confirmed in in vivo models for the particular disease condition under study.

First, a suitable in vivo model is selected. For example, mouse tumor models are available for many types of cancers, including cancers with specific metastasis patterns, and can be selected from known sources such as the Mouse Tumor Biology Database Project (MTBDP), which acts as a clearinghouse for information on mouse tumor models available. The MTBDP is available on the internet at tumor.informaticsjax.org/FMPro?-db=TumorInstance&-format=mtdp.html&-view. Mouse inflammation and diabetes models are available from, for example, The Jackson Laboratory (Bar Harbor, Me.) under the name Jax Mice & Services; see jax.org/jaxmice and jaxmice.jax.org/jaxmicedb/html/sbmodel_{—}7.shtml, respectively. Other disease models, in mice or in other mammals, may be used, as well. For example, mouse, hamster or rat models for arthritis and obesity are known.

As a second example, the anti-inflammatory activity of a presumptive single agent may be assessed using a carrageenan-induced arthritis rat model. Carrageenan-induced arthritis has also been used in rabbit, dog and pig in studies of chronic arthritis or inflammation. Quantitative histomorphometric assessment is used to determine therapeutic efficacy. The methods for using such a carrageenan-induced arthritis model is described in Hansra P. et al., “Carrageenan-Induced Arthritis in the Rat,” Inflammation, 24(2): 141-155, (2000). Also commonly used are zymosan-induced inflammation animal models as known and described in the art. The anti-inflammatory activity of presumptive single agent can also be assessed by measuring the inhibition of carrageenan-induced paw edema in the rat, using a modification of the method described in Winter C. A. et al., “Carrageenan-Induced Edema in Hind Paw of the Rat as an Assay for Anti-inflammatory Drugs” Proc. Soc. Exp. Biol Med. 111, 544-547 (1962). This assay has been used as a primary in vivo screen for the anti-inflammatory activity of most NSAIDs, and is considered predictive of human efficacy. The anti-inflammatory activity of the test presumptive single agent is expressed as the percent inhibition of the increase in hind paw weight of the test group relative to the vehicle dosed control group.

Animal models for asthma can also be used to assess the efficacy of a presumptive single agent. see, e.g., Cohn et al., 1997, J. Exp. Med. 1861737-1747).

Animal models for autoimmune disorders can also be used to assess the efficacy of a presumptive single agent. Animal models for autoimmune disorders such as type 1 diabetes, thyroid autoimmunity, systemic lupus erythematosus, and glomerulonephritis have been developed (Flanders et al., 1999, Autoimmunity 29:235-246; Krogh et al., 1999, Biochimie 81:511-515; Foster, 1999, Semin. Nephrol. 19:12-24).

Alternatively, in vivo models may be created. For example, tumor models may be created by administration of specific tumor cells to mice. As an example, a colon carcinoma model may be constructed as follows. MCA26 is a tumor cell line of chemically induced colon carcinoma in BALB/c mouse (Corbett et al., 1975). Metastatic colon cancer is induced by implanting approximately 7×10⁴ MCA26 cells into the left lobe of the liver of 8-10 week old female BALB/c mice (Taconic). At day 7, mice with 5×5 mm² size tumors are selected for administration of the presumptive single agent. Alternatively, cells may be administered intraperitoneally or through the tail vein.

Once the model is selected, specific kinases are identified as potential targets. For example, for tumor models, appropriate kinases to target may be those known to be involved in cell cycle control, attachment signaling, or angiogenesis. Kinases with unknown, or poorly-characterized, roles in a particular disease condition may also be tested; however, where this is the case, any inhibition or modulation of kinase activity must be correlated with the appropriate and desired response (e.g., reduction in proliferation).

Particular compounds (i. e., presumptive single agents) are then tested for their ability to affect the disease condition under study. The agent to be tested is administered to the subject animal, and the physiological response of the disease condition is compared to a control (typically an animal to which is administered only the carrier for the agent). Criteria for efficacy depend upon the disease condition under consideration. For example, typical efficacy criteria for tumor models is a reduction in apparent weight loss, reduction or lack of increase in tumor size; reduction in metastasis; decreased numbers of abnormal cells in tissue slices, and the like. Criteria may be subjective, such as an apparent improvement in appearance or health, or objective, in which case the difference(s) between control and experimental groups is typically statistically determined. Kinase activities, and changes in activities, can be determined by taking blood or tissue samples by standard means, and testing kinase activity using one or more of the example assays provided in Examples 4 to 83.

Single agents are identified in such in vivo assays as agents that affect two or more kinases, and have at least one beneficial effect on the disease condition under study.

Examples 4-83: Assays for measuring activity of candidates: The following assays may be used to assess kinase activity; the assays are modified in the experimental condition by the addition of a compound to be tested for kinase inhibitory or modulatory activity. These example assays are not meant to be exclusive; the activity of various kinases may be assessed by other methods, as well.

### 6.4 Example 4

### JNK2 Assay

To 10 μL of the test compound in 20% DMSO/80% dilution buffer consisting of 20 mM HEPES (pH 7.6), 0.1 mM EDTA, 2.5 mM magnesium chloride, 0.004% Triton ×100, 2 μg/mL leupeptin, 20 mM glycerolphosphate, 0.1 mM sodium vanadate, and 2 mM DTT in water is added 30 μL of 50 ng His6-JNK2 in the same dilution buffer. The mixture is preincubated for 30 minutes at room temperature. Sixty microliter of 10 μg GST-c-Jun(1-79) in assay buffer consisting of 20 mM HEPES (pH 7.6), 50 mM sodium chloride, 0.1 mM EDTA, 24 mM magnesium chloride, 1 mM DTT, 25 mM PNPP, 0.05% Triton ×100, 11 μM ATP, and 0.5 μCi γ-³²P ATP in water is added and the reaction is allowed to proceed for 1 hour at room temperature. The c-Jun phosphorylation is terminated by addition of 150 μL of 12.5% trichloroacetic acid. After 30 minutes, the precipitate is harvested onto a filter plate, diluted with 50 tL of the scintillation fluid and quantified by a counter. The IC₅₀ values are calculated as the concentration of the test compound at which the c-Jun phosphorylation is reduced to 50% of the control value. Preferred compounds of the present invention have an IC₅₀ value ranging 0.01-10 μM in this assay.

### 6.5 Example 5

### JNK3 Assay

To 10 μL of the test compound in 20% DMSO/80% dilution buffer consisting of 20 mM HEPES (pH 7.6), 0.1 mM EDTA, 2.5 mM magnesium chloride, 0.004% Triton ×100, 2 μg/mL leupeptin, 20 mM glycerolphosphate, 0.1 mM sodium vanadate, and 2 mM DTT in water is added 30 μL of 200 ng His6-JNK3 in the same dilution buffer. The mixture is preincubated for 30 minutes at room temperature. Sixty microliter of 10 μg GST-c-Jun(1-79) in assay buffer consisting of 20 mM HEPES (pH 7.6), 50 mM sodium chloride, 0.1 mM EDTA, 24 mM magnesium chloride, 1 mM DTT, 25 mM PNPP, 0.05% Triton ×100, 11 μM ATP, and 0.5 μCi γ-³²P ATP in water is added and the reaction is allowed to proceed for 1 hour at room temperature. The c-Jun phosphorylation is terminated by addition of 150 μL of 12.5% trichloroacetic acid. After 30 minutes, the precipitate is harvested onto a filter plate, diluted with 50 μL of the scintillation fluid and quantified by a counter. The IC₅₀ values are calculated as the concentration of the test compound at which the c-Jun phosphorylation is reduced to 50% of the control value. Preferred compounds of the present invention have an IC₅₀ value ranging 0.001-10 μM in this assay.

### 6.6 Example 6

### Jurkat T-cell IL-2 Production Assay

Jurkat T cells (clone E6-1) are purchased from the American Tissue Culture Collection and maintained in growth media consisting of RPMI 1640 medium containing 2 mM L-glutamine (Mediatech), with 10% fetal bovine serum (Hyclone) and penicillin/streptomycin. All cells are cultured at 37° C. in 95% air and 5% CO₂. Cells are plated at a density of 0.2×10 cells per well in 200 μL of media. Compound stock (20 mM) is diluted in growth media and added to each well as a 10× concentrated solution in a volume of 25 μL, mixed, and allowed to pre-incubate with cells for 30 minutes. The compound vehicle (dimethylsulfoxide) is maintained at a final concentration of 0.5% in all samples. After 30 minutes the cells are activated with PHA (phorbol myristate acetate; final concentration 50 μg/mL) and PHA (phytohemagglutinin; final concentration 2 μg/mL). PMA and PHA are added as a 10× concentrated solution made up in growth media and added in a volume of 25 μL per well. Cell plates are cultured for 10 hours. Cells are pelleted by centrifugation and the media removed and stored at −20° C. Media aliquots are analyzed by sandwich ELISA for the presence of IL-2 as per the manufacturers instructions (Endogen). The IC₅₀ values are calculated as the concentration of the test compound at which the IL-2 production was reduced to 50% of the control value. Preferred compounds of the present invention have an IC₅₀ value ranging 0.01-10 μM in this assay.

### 6.7 Example 7

### Rat in vivo LPS-induced TNF-α Production Assay

Male CD rats procured from Charles River Laboratories at 7 weeks of age are allowed to acclimate for one week prior to use. A lateral tail vein is cannulated percutaneously with a 22-gage over-the-needle catheter under brief isoflurane anesthesia. Rats are administered test compound either by intravenous injection via the tail vein catheter or oral lavage 15 to 180 min prior to injection of 0.05 mg/kg LPS (Escherichia coli 055:BS). Catheters are flushed with 2.5 mL/kg of normal injectable saline. Blood is collected via cardiac puncture 90 minutes after LPS challenge. Plasma is prepared using lithium heparin separation tubes and frozen at −80° C. until analyzed. TNF-α levels are determined using a rat specific TNF-( ELISA kit (Biosource). The ED₅₀ values are calculated as the dose of the test compound at which the TNF-α production is reduced to 50% of the control value. Preferred compounds of the present invention have an ED₅₀ value ranging 1-30 mg/kg in this assay.

### 6.8 Example 8

### CDC Kinase Activity Assay

Cyclin-dependent kinase activity can be measured by quantifying the enzyme-catalyzed, time-dependent incorporation of radioactive phosphate from [³²P]ATP or [³³P]ATP into a protein substrate. Unless noted otherwise, assays are performed in 96-well plates in a total volume of 50 μL, in the presence of 10 mM HEPES (N-[2-hydroxyethyl]piperazine-N′-[2-ethanesulfonic acid]) (pH 7.4), 10 mM MgCl₂, 25 μM adenosine triphosphate (ATP), 1 mg/mL ovalbumin, 5 μg/mL leupeptin, 1 mM dithiothreitol, 10 mM beta-glycerophosphate, 0.1 mM sodium vanadate, 1 mM sodium fluoride, 2.5 mM ethylene glycol-bis(β-aminoethyl ethKer)-N,N,N′N′-tetraacetic acid (EGTA), 2% (v/v) dimethylsulfoxide, and 0.03-0.4 μCi [^{32/33}P]ATP per reaction. Reactions are initiated with appropriate enzyme, incubated at 30° C., and terminated after 20 minutes by the addition of ethylenediaminetetraacetic acid (EDTA) to 250 mM. The phosphorylated substrate is then captured on a nitrocellulose or phosphocellulose membrane using a 96-well filtration manifold, and unincorporated radioactivity is removed by repeated washing with 0.85% phosphoric acid. Radioactivity is quantified by exposing the dried membranes to a phosphorimager.

Apparent Kᵢ values are measured by assaying enzyme activity in the presence of different inhibitor compound concentrations and subtracting the background radioactivity measured in the absence of enzyme. Inhibition data are fit to an equation for competitive inhibition using Kaleidagraph (Synergy Software), or are fit to an equation for competitive tight-binding inhibition using the software KineTic (BioKin, Ltd.).

### 6.9 Example 9

### Inhibition of CDK4/Cyclin D Retinoblastoma Kinase Activity

A complex of human CDK4 and cyclin D3, or a complex of human CDK4 and genetically truncated (1-264) cyclin D3, is purified using traditional biochemical chromatographic techniques from insect cells that had been co-infected with the corresponding baculovirus expression vectors (see e.g., Meijer and Kim, “Chemical Inhibitors of Cyclin-Dependent Kinases,” Methods in Enzymol., vol. 283 (1997), pp. 113-128). The enzyme complex (5 or 50 nM) is assayed with 0.3-0.5 μg of purified recombinant retinoblastoma protein fragment (Rb) as a substrate. The engineered Rb fragment (residues 386-928 of the native retinoblastoma protein; 62.3 kDa) contains the majority of the phosphorylation sites found in the native 106-kDa protein, as well as a tag of six histidine residues for ease of purification. Phosphorylated Rb substrate is captured by microfiltration on a nitrocellulose membrane and quantified using a phosphorimager as described above. For measurement of tight-binding inhibitors, the enzyme complex concentration is lowered to 5 nM, and the assay duration is extended to 60 minutes, during which the time-dependence of product formation is linear.

### 6.10 Example 10

### Inhibition of CDK2/Cyclin A Retinoblastoma Kinase Activity

CDK2 is purified using the methodology described in Rosenblatt et al., “Purification and Crystallization of Human Cyclin-dependent Kinase 2, ” J. Mol. Biol., vol. 230, 1993, pp. 1317-1319. Cyclin A is purified from E. coli cells expressing full-length recombinant cyclin A, and a truncated cyclin A construct is generated by limited proteolysis and purified as described in Jeffrey et al., “Mechanism of CDK activation revealed by the structure of a cyclin A-CDK2 complex,” Nature, vol. 376 (Jul. 27, 1995), pp. 313-320. A complex of CDK2 and proteolyzed cyclin A is prepared and purified by gel filtration. The substrate for this assay is the same Rb substrate fragment used for the CDK4 assays, and the methodology of the CDK2/cyclin A and the CDK4/cyclin D3 assays is essentially the same, except that CDK2 is present at 150 nM or 5 nM. Kᵢ values are measured as described above.

### 6.11 Example 11

### VEGF Activity Assay

The stimulation of cell proliferation by growth factors such as VEGF and others is dependent upon their induction of autophosphorylation of each of their respective receptor's tyrosine kinases. Therefore, the ability of a protein kinase inhibitor to block cellular proliferation induced by these growth factors is directly correlated with its ability to block receptor autophosphorylation. To measure the protein kinase inhibition activity of the compounds, the following constructs are used.

VEGF-R2 Construct for Assay: This construct determines the ability of a test compound to inhibit tyrosine kinase activity. A construct (VEGF-R2Δ50) of the cytosolic domain of human vascular endothelial growth factor receptor 2 (VEGF-R2) lacking the 50 central residues of the 68 residues of the kinase insert domain is expressed in a baculovirus/insect cell system. Of the 1356 residues of full-length VEGF-R2, VEGF-R2Δ50 contains residues 806-939 and 990-1171, and also one point mutation (E990V) within the kinase insert domain relative to wild-type VEGF-R2. Autophosphorylation of the purified construct is performed by incubation of the enzyme at a concentration of 4 μM in the presence of 3 mM ATP and 50 mM MgCl₂ in 100 mM Hepes, pH 7.5, containing 5% glycerol and 5 mM DTT, at 4° C. for 2 hours. After autophosphorylation, this construct has been shown to possess catalytic activity essentially equivalent to the wild-type autophosphorylated kinase domain construct. See Parast et al., Biochemistry, 37, 16788-16801(1998).

CHK1 Construct for Assay: C-terminally His-tagged full-length human CHK1 (FL-CHK1) is expressed using the baculovirus/insect cell system. It contains 6 histidine residues (6×His-tag) at the C-terminus of the 476 amino acid human CHK1. The protein is purified by conventional chromatographic techniques.

VEGF-R2 Assay—Coupled Spectrophotometric (FLVK-P) Assay: The production of ADP from ATP that accompanies phosphoryl transfer is coupled to oxidation of NADH using phosphoenolpyruvate (PEP) and a system having pyruvate kinase (PK) and lactic dehydrogenase (LDH). The oxidation of NADH can be monitored by following the decrease of absorbance at 340 nm (e₃₄₀=6.22 cm⁻¹ mM⁻¹) using a Beckman DU 650 spectrophotometer. Assay conditions for phosphorylated VEGF-R2Δ50 (indicated as FLVK-P in the tables below) are as follows: 1 mM PEP; 250 μM NADH; 50 units of LDH/mL; 20 units of PK/mL; 5 mM DTT; 5.1 mM poly(E₄Y₁); 1 mM ATP; and 25 mM MgCl₂ in 200 mM Hepes, pH. 7.5. Assay conditions for unphosphorylated VEGF-R2Δ50 (indicated as FLVK in the tables) are as follows: 1 mM PEP; 250 μM NADH; 50 units of LDH/mL; 20 units of PK/mL; 5 mM DTT; 20 mM poly(E₄Y₁); 3 mM ATP; and 60 mM MgCl₂ and 2 mM MnCl₂ in 200 mM Hepes, pH 7.5. Assays are initiated with 5 to 40 nM of enzyme. Ki values are determined by measuring enzyme activity in the presence of varying concentrations of test compounds. The data are analyzed using Enzyme Kinetic and Kaleidagraph software.

### 6.12 Example 12

### CHK1 Assay

The production of ADP from ATP that accompanies phosphoryl transfer to the synthetic substrate peptide Syntide-2 (PLARTLSVAGLPGKK; SEQ ID NO:1) is coupled to oxidation of NADH using phosphoenolpyruvate (PEP) through the actions of pyruvate kinase (PK) and lactic dehydrogenase (LDH). The oxidation of NADH can be monitored by following the decrease of absorbance at 340 nm (ε340-6.22 cm⁻¹ mM⁻¹) using a HP8452 spectrophotometer. Typical reaction solutions contain: 4 mN PEP; 0.15 mM NADH; 28 units of LDH/ml; 16 units of PK/ml; 3 mM DTT; 0.125 mM Syntide-2; 0.15 mM ATP; 25 mM MgCl₂ in 50 mM TRIS, pH 7.5; and 400 mM NaCl. Assays are initiated with 10 nM of FL-CHK1. Kᵢ values are determined by measuring initial enzyme activity in the presence of varying concentrations of test compounds. The data are analyzed using Enzyme Kinetic and Kaleidagraph software.

### 6.13 Example 13

### Inhibition of Phosphorylated FGF Receptor and LCK Tyrosine Kinase Activity

Cloning, expression and purification of the cytosolic domain of FGFR¹ tyrosine kinase (amino acids 456-766) containing three amino acid substitutions (L457V, C488A, and C84S) can be conducted as described in Mohammadi, M. Schlessinger, J., & Hubbard, S. R. (1996) Cell 86, 577-587. This domain is expressed in Sf9 insect cells using a baculovirus expression vector, and protein is purified using conventional techniques. The LCK tyrosine kinase is expressed in insect cells as an N-terminal deletion starting from amino acid 223 to the end of the protein at amino acid 509. The N-terminus of the protein also had two amino acid substitutions, P223M and c 224D. Kinases are purified using conventional chromatographic methods.

Tyrosine kinase activity can be measured using a coupled, continuous spectrophotometric assay, in which production of phosphorylated poly(Glu, Tyr; 4:1) substrate and ADP is coupled to the pyruvate kinase-catalyzed transfer of a phosphate from phosphoenolpyruvate to ADP, with generation of pyruvate and regeneration of ATP. Pyruvate production is in turn coupled to the lactate dehydrogenase-catalyzed reduction of pyruvate to form lactate, with concomitant conversion of NADH to NAD⁺. Loss of NADH is monitored by measuring absorbance at 340 nm (see e.g., Technikova-Dobrova et al., “Spectrophotometric determination of finctional characteristics of protein kinases with coupled enzymatic assay,” FEBS Letters, vol. 292 (1991), pp. 69-72). Enzyme activity is measured in the presence of 200 mM HEPES (pH 7.5), 2 mM phosphoenolpyruvate, 0.3 mM NADH, 20 mM MgCl₂, 100 μM ATP, 5 mM DTT, 5.1 or 25 mM poly (Glu, Tyr) 4:1 for P-FGF or P-LCK assays, respectively, and 15 units/mL each of pyruvate kinase and lactate dehydrogenase. Phosphorylated FGF receptor kinase is present at 100 nM and phosphorylated LCK kinase is present at 50 nM. Assays are performed under initial rate conditions at 37° C., and rates are corrected for any background rate measured in the absence of enzyme. Percent inhibition is calculated relative to control enzyme assayed in the presence of 2% (v/v) DMSO.

### 6.14 Example 14

### General Kinase Inhibition Assay

Inhibition of various kinases was monitored by examining the transfer of phosphate to an appropriate peptide substrate for each kinase.

Activities for Jnk1, Jnk2, Jnk3, IKK1, IKK2EE, p38a, p38p, MKK3, MKK4, MKK6, MKK7, cdk2/E, cdk2/A, PKCα, ERK and PKA were monitored by the transfer of radio-labeled phosphate from ATP(γSSP) to a protein substrate, and precipitation of the product using trichloroacetic acid. ATP was at 3 times the Km for the relevant kinase. Activities for Akt1, Akt2, and SGK were monitored by the transfer of radio-labeled phosphate from ATP to a specific substrate peptide and capture of the peptide on P81 charged filter paper. ATP was at the Km for the relevant kinase. Activities for IRTK, Abl, and SRC were monitored by transfer of phosphate from ATP to a biotinylated peptide substrate and detection of the phosphorylated peptide using the LANCE technology (Perkin Elmer). ATP was at 3 times the Km for the relevant kinase.

Agents that target two or more different kinases are useful in the methods of the invention as described elsewhere herein. Such compounds may be identified, and their effect on certain kinases may be identified, using the assays briefly described below in Examples 4-83. Each of these assays was carried out as described in Davies et al., Biochem. J., 351:95-105 (2000). All assays were carried out at 10 μM ATP unless otherwise noted.

Kinase Dilution

All kinases are pre-diluted to a 10× working concentration prior to addition into the assay. The composition of the dilution buffer for each kinase is detailed below.

In addition, the following abbreviations are used: h is human; r is rat; m is mouse; b is bovine; and y is yeast.

TABLE 6
Buffer Compositions Used In Various Kinase Assays

| Buffer composition | Kinase(s) |
|---|---|
| 50 mM Tris pH 7.5, 0.1 mM EGTA, | Blk, c-RAF, CSK, FGFR3, IGF- |
| 0.1 mM NaVanadate, 0.1% β- | 1R, IR, Lyn, MAPK1, MAPK2, |
| mercaptoethanol, 1 mg/ml BSA | MKK4, MKK6, MKK7β, |
| | SAPK2a, SAPK2b, SAPK3, |
| | SAPK4, Syk, and ZAP-70 |
| 50 mM Tris pH 7.5, 0.1 mM EGTA, | JNK1α1, JNK2α2, JNK3, PRK2, |
| 0.1% β-mercaptoethanol, 1 mg/ml | and ROCK-II |
| BSA | |
| 50 mM Tris pH 7.5, 0.05% β- | PDK1 |
| mercaptoethanol, 1 mg/ml | |
| BSA | |
| 25 mM Tris pH 7.5, 0.1 mM EGTA, | MEK1 |
| 0.1% β-mercaptoethanol, 1 mg/ml | |
| BSA | |
| 20 mM MOPS pH 7.0, 1 mM EDTA, | ABL, CDK1/cyclinB, |
| 0.1% β-mercaptoethanol, 0.01% | CDK2/cyclinA, CDK2/cyclinE, |
| Brij-35, 5% glycerol, 1 mg/ml BSA | CDK3/cyclinE, CDK5/p35, |
| | CDK6/cyclinD3, CDK7/cyclinH/ |
| | MAT1, CHK1, CHK2, CK1, |
| | cSRC, Fes, Fyn, GSK3β, IKKα, |
| | IKKβ, Lck, MAPKAP-K2, MSK1, |
| | p70S6K, PAK2, PDGFRα, |
| | PDGFRβ, PKA, PKBα, PKBβ, |
| | PKCθ, Rsk1, Rsk2, Rsk3, SGK, |
| | and Yes |
| 20 mM Hepes pH 7.4, 0.15 M NaCl, | CK2 |
| 0.1 mM EGTA, 5 mM DTT, 0.1% | |
| Triton X-100, 50% glycerol | |
| 180 mM Hepes pH 7.4, 3.6 mM | AMPK |
| DTT, 0.07% Brij-35 | |
| 40 mM Hepes pH 7.4, 1 mg/ml BSA | CaMKII, CaMKIV |
| 20 mM Hepes pH 7.4, 0.03% Triton | PKCα, PKCβII, PKCγ, PKCε |
| X-100 | |
| 20 mM Na-β-glycerophosphate | PRAK |
| pH 7.5, 0.1% β-mercaptoethanol, | |
| 0.1 mM EGTA, 1 mg/ml BSA | |

Substrates

All substrates are dissolved and diluted to working stocks in de-ionised water, apart from histone H1, which is diluted to a 10× working stock in 20 mM MOPS pH 7.4 prior to addition into the assay, and ATF2 which is typically stored at a 20× working stock in 50 mM Tris pH 7.5, 150 mM NaCl, 0.1 mM EGTA, 0.03% Brij-35, 50% glycerol, 1 mM benzamidine, 0.2 mM PMSF and 0.1% β-mercaptoethanol.

### 6.15 Example 15

### SGK(h) Assay

In a final reaction volume of 25 μl, SGK(h) (5-10 mU) is incubated with 8 mM MOPS pH 7.0, 0.2 mM EDTA, 30 μM GRPRTSSFAEGKK (SEQ ID NO:2), 10 mM MgAcetate and [γ-³³P-ATP] (specific activity approx. 500 cpm/pmol, concentration as required). The reaction is initiated by the addition of Mg²⁺[γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.16 Example 16

### GSK3p (h) Assay

In a final reaction volume of 25 μl, GSK3β (h) (5-10 mU) is incubated with 8 mM MOPS pH 7.0, 0.2 mM EDTA, 20 μM YRRAAVPPSPSLSRHSSPHQS(p)EDEEE (phospho GS2 peptide; SEQ ID NO:3), 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

### 6.17 Example 17

### AMPK(r) Assay

In a final reaction volume of 25 μl, AMPK(r) (5-10 mU) is incubated with 50 mM Hepes pH 7.4, 1 mM DTT, 0.02% Brij-35, 200 μM AMP, 200 μM AMARAASAAALARRR (SEQ ID NO:4), 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.18 Example 18

### CHK1(h) Assay

In a final reaction volume of 25 μl, CHK1(h) (5-10 mU) is incubated with 8 mM MOPS pH 7.0,0.2 mM EDTA, 200 μM KKKVSRSGLYRSPSMPENLNRPR (SEQ ID NO:5), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.19 Example 19

### CK2(h) Assay

In a final reaction volume of 25 μl, CK2(h) (5-10 mU) is incubated with 20 mM Hepes pH 7.6, 0.15 M NaCl, 0.1 mM EDTA, 5 mM DTT, 0.1% Triton X-100, 165 μM RRRDDDSDDD (SEQ ID NO:6), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.20 Example 20

### Lck(h) Assay

In a final reaction volume of 25 μl, Lck(h) (5-10 mU) is incubated with 50 mM Tris pH 7.5, 0.1 mM EGTA, 0.1 mM NaVanadate, 250 μM KVEKIGEGTYGVVYK (Cdc2 peptide; SEQ ID NO:7), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.21 Example 21

### CDK2/cyclinA (h) Assay

In a final reaction volume of 25 μl, CDK2/cyclinA (h) (5-10 mU) is incubated with 8 mM MOPS pH 7.0, 0.2 mM EDTA, 0.1 mg/ml histone H1, 10 mM MgAcetate and [γ-³³p-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.22 Example 22

### MAPK2 (m) Assay

In a final reaction volume of 25 μl, MAPK2 (m) (5-10 mU) is incubated with 25 mM Tris pH 7.5, 0.02 mM EGTA, 0.33 mg/ml myelin basic protein, 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.23 Example 23

### SAPK2a (h) Assay

In a final reaction volume of 25 μl, SAPK2a (h) (5-10 mU) is incubated with 25 mM Tris pH 7.5, 0.02 mM EGTA, 0.33 mg/ml myelin basic protein, 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.24 Example 24

### SAPK2b (h) Assay

In a final reaction volume of 25 μl, SAPK2b (h) (5-10 mU) is incubated with 25 mM Tris pH 7.5, 0.02 mM EGTA, 0.33 mg/ml myelin basic protein, 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.25 Example 25

### SAPK3 (h) Assay

In a final reaction volume of 25 μl, SAPK3 (h) (5-10 mU) is incubated with 25 mM Tris pH 7.5, 0.02 mM EGTA, 0.33 mg/ml myelin basic protein, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.26 Example 26

### SAPK4 (h) Assay

In a final reaction volume of 25 μl, SAPK4 (h) (5-10 mU) is incubated with 25 mM Tris pH 7.5, 0.02 mM EGTA, 0.33 mg/ml myelin basic protein, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.27 Example 27

### MSK1 (h) Assay

In a final reaction volume of 25 μl, MSK1 (h) (5-10 mU) is incubated with 8 mM MOPS pH 7.0, 0.2 mM EDTA, 30 μM GRPRTSSFAEGKK (SEQ ID NO:2), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.28 Example 28

### PKBA (h) Assay

In a final reaction volume of 25 μl, PKBα (h) (5-10 mU) is incubated with 8 mM MOPS pH 7.0, 0.2 mM EDTA, 30 μM GRPRTSSFAEGKK (SEQ ID NO:2), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.29 Example 29

### ROCK-II (r) Assay

In a final reaction volume of 25 μl, ROCK-II (r) (5-10 mU) is incubated with 50 mM Tris pH 7.5, 0.1 mM EGTA, 30 μM KEAKEKRQEQIAKRRRLSSLRASTSKSGGSQK (SEQ ID NO:8), 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.30 Example 30

### p70S6K (h) Assay

In a final reaction volume of 25 μl, p70S6K (h) (5-10 mU) is incubated with 8 mM MOPS pH 7.0, 0.2 mM EDTA, 100 μM KKRNRTLTV (SEQ ID NO:9), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.31 Example 31

### PKA (b) Assay

In a final reaction volume of 25 μl, PKA (b) (5-10 mU) is incubated with 8 mM MOPS pH 7.0, 0.2 mM EDTA, 30 μM LRRASLG (Kemptide; SEQ ID NO: 10), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

### 6.32 Example 32

### MAPKAP-K2 (h) Assay

In a final reaction volume of 25 μl, MAPKAP-K2 (h) (5-10 mU) is incubated with 50 mM Na-β-glycerophosphate pH 7.5, 0.1 mM EGTA, 30 μM KKLNRTLSVA (SEQ ID NO:11), 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.33 Example 33

### JNK1α1 (h) Assay

In a final reaction volume of 25 μl, JNK1α1 (h) (5-10 mU) is incubated with 50 mM Tris pH 7.5, 0.1 mM EGTA, 0.1% β-mercaptoethanol, 3 μM ATF2, 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.34 Example 34

### JNK2α2 (h) Assay

In a final reaction volume of 25 μl, JNK2α2 (h) (5-10 mU) is incubated with 50 mM Tris pH 7.5, 0.1 mM EGTA, 0.1% β-mercaptoethanol, 3 μM ATF2, 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.35 Example 35

### JNK3 (r) Assay

In a final reaction volume of 25 μl, JNK3 (r) (5-10 mU) is incubated with 50 mM Tris pH 7.5, 0.1 mM EGTA, 0.1% β-mercaptoethanol, 250 μM peptide, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.36 Example 36

### PRAK (h) Assay

In a final reaction volume of 25 μl, PRAK (h) (5-10 mU) is incubated with 50 mM Na-β-glycerophosphate pH 7.5, 0.1 mM EGTA, 30 μM KKLRRTLSVA (SEQ ID NO11), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

### 6.37 Example 37

### CHK2 (h) Assay

In a final reaction volume of 25 μl, CHK2 (h) (5-10 mU) is incubated with 8 mM MOPS pH 7.0,0.2 mM EDTA, 200 μM KKKVSRSGLYRSPSMPENLNRPR (SEQ ID NO:5), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.38 Example 38

### MAPK1 (h) Assay

In a final reaction volume of 25 μl, MAPK1 (h) (5-10 uM) is incubated with 25 mM Tris pH 7.5, 0.02 mM EGTA, 250 μM peptide, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.39 Example 39

### c-RAF (h) Assay

In a final reaction volume of 25 μl, c-RAF (h) (5-10 mU) is incubated with 25 mM Tris pH 7.5, 0.02 mM EGTA, 0.66 mg/ml myelin basic protein, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.40 Example 40

### CDK1/cyclinB (h) Assay

In a final reaction volume of 25 μl, CDK1/cyclinB (h) (5-10 mU) is incubated with 8 mM MOPS pH 7.0, 0.2 mM EDTA, 0.1 mg/ml histone H1, 10 mM MgAcetate and [γ-³³p-ATP] (specific activity approx. 500 cpm/pmol, concentration as required). The reaction is initiated by the addition of Mg²⁺ [γ-33 P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.41 Example 41

### cSRC (h) Assay

In a final reaction volume of 25 μl, cSRC (h) (5-10 mU) is incubated with 8 mM MOPS pH 7.0, 0.2 mM EDTA, 250 μM KVEKIGEGTYGVVYK (Cdc2 peptide; SEQ ID NO:7), 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.42 Example 42

### CaMKII (r) Assay

In a final reaction volume of 25 μl, CaMKII (r) (5-10 mU) is incubated with 40 mM Hepes pH 7.4, 5 mM CaCl₂, 30 μg/ml calmodulin, 30 μM KKLNRTLSVA (SEQ ID NO:11), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.43 Example 43

### PRK2 (h) Assay

In a final reaction volume of 25 μl, PRK2 (h) (5-10 mU) is incubated with 50 mM Tris pH 7.5, 0.1 mM EGTA, 0.1% β-mercaptoethanol, 30 μM AKRRRLSSLRA (SEQ ID NO:12), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.44 Example 44

### PDKM (h) Assay

In a final reaction volume of 25 μl, PDK1 (h) (5-10 mU) is incubated with 50 mM Tris pH 7.5, 100 μM KTFCGTPEYLAPEVRREPRILSEEEQEMFRDFDYIADWC (SEQ ID NO:13) (PDKtide), 0.1% β-mercaptoethanol, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.45 Example 45

### Fyn (h) Assay

In a final reaction volume of 25 μl, Fyn (h) (5-10 mU) is incubated with 50 mM Tris pH 7.5, 0.1 mM EGTA, 0.1 mM NaVanadate, 250 μM KVEKIGEGTYGVVYK (Cdc2 peptide; SEQ ID NO:7), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.46 Example 46

### PKCα (h) Assay

In a final reaction volume of 25 μl, PKCα (h) (5-10 mU) is incubated with 20 mM Hepes pH 7.4, 0.03% Triton X-100, 0.1 mM CaCl₂, 0.1 mg/ml phosphatidylserine, 10 μg/ml diacylglycerol, 0.1 mg/ml histone H1, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.47 Example 47

### PKCβII (h) Assay

In a final reaction volume of 25 μl, PKCβII (h) (5-10 mU) is incubated with 20 mM Hepes pH 7.4, 0.03% Triton X-100, 0.1 mM CaCl₂, 0.1 mg/ml phosphatidylserine, 10 μg/ml diacylglycerol, 0.1 mg/ml histone H1, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 1 of the reaction is then spotted onto a P30 filtermat and washed three times for 5 minutes in 75 mM phosphoric acid and once in methanol prior to drying and scintillation counting.

### 6.48 Example 48

### PKCγ (h) Assay

In a final reaction volume of 25 μl, PKCγ (h) (5-10 mU) is incubated with 20 mM Hepes pH 7.4, 0.03% Triton X-100, 0.1 mM CaCl₂, 0.1 mg/ml phosphatidylserine, 10 μg/ml diacylglycerol, 0.1 mg/ml histone H1, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.49 Example 49

### CK1 (y) Assay

In a final reaction volume of 25 μl, CK1 (y) (5-10 mU) is incubated with 8 mM MOPS pH 7.0,0.2 mM EDTA, 200 μM KRRRALS(p)VASLPGL (SEQ ID NO:14), 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.50 Example 50

### ZAP-70 (h) Assay

In a final reaction volume of 25 μl, ZAP-70 (h) (5-10 mU) is incubated with 50 mM Tris pH 7.5, 0.1 mM EGTA, 0.1 mM NaVanadate, 0.1% β-mercaptoethanol, 0.1 mg/ml poly(Glu, Tyr) 4:1, 10 mM MnCl₂, 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

### 6.51 Example 51

### MEK1 (h) Assay

In a final reaction volume of 25 μl, MEK1 (h) (1-5 mU) is incubated with 50 mM Tris pH 7.5, 0.2 mM EGTA, 0.1% ,-mercaptoethanol, 0.01% Brij-35, 1 μM inactive MAPK2 (m), 10 mM MgAcetate and cold ATP (concentration as required). The reaction is initiated by the addition of the MgATP. After incubation for 40 minutes at room temperature, 5 μl of this incubation mix is used to initiate a MAPK2 (m) assay, which is described on page 7 of this book.

### 6.52 Example 52

### MKK4 (m) Assay

In a final reaction volume of 25 μl, MKK4 (m) (1-5 mU) is incubated with 50 mM Tris pH 7.5, 0.1 mM EGTA, 0.1% β-mercaptoethanol, 0.1 mM NaVanadate, 2 μM inactive JNK1α1 (h), 10 mM MgAcetate and cold ATP (concentration as required). The reaction is initiated by the addition of the MgATP. After incubation for 40 minutes at room temperature, 5 μl of this incubation mix is used to initiate a JNK1α1 (h) assay, which is exactly as described on page 10 of this book except that ATF2 is replaced with 250 μM peptide.

### 6.53 Example 53

### MKK7β (h) Assay

In a final reaction volume of 25 μl, MKK7β (h) (1-5 mU) is incubated with 50 mM Tris pH 7.5, 0.1 mM EGTA, 0.1% β-mercaptoethanol, 0.1 mM NaVanadate, 2 μM inactive JNK1α1 (h), 10 mM MgAcetate and cold ATP (concentration as required). The reaction is initiated by the addition of the MgATP. After incubation for 40 minutes at room temperature, 5 μl of this incubation mix is used to initiate a JNK1α1 (h) assay, which is exactly as described on page 10 of this book except that ATF2 is replaced with 250 μM peptide.

### 6.54 Example 54

### MKK6 (h) Assay

In a final reaction volume of 25 μl, MKK6 (h) (1-5 mU) is incubated with 50 mM Tris pH 7.5, 0.1 mM EGTA, 0.1% β-mercaptoethanol, 0.1 mM NaVanadate, 1 mg/ml BSA, 1 jM inactive SAPK2a (h), 10 mM MgAcetate and cold ATP (concentration as required). The reaction is initiated by the addition of the MgATP. After incubation for 40 minutes at room temperature, 5 μl of this incubation mix is used to initiate a SAPK2a (h) assay, which is described on page 8 of this book.

### 6.55 Example 55

### IKKα (h) Assay

In a final reaction volume of 25 μl, IKKα (h) (5-10 mU) is incubated with 8 mM MOPS pH 7.0, 0.2 mM EDTA, 200 μM peptide, 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.56 Example 56

### IKKβ (h) Assay

In a final reaction volume of 25 μl, IKKβ (h) (5-10 mU) is incubated with 8 mM MOPS pH 7.0, 0.2 mM EDTA, 100 μM peptide, 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.57 Example 57

### PKCθ (h) Assay

In a final reaction volume of 5 μl, PKCθ (5-10 mU) is incubated with 8 mM MOPS pH 7.0, 0.2 mM EDTA, 0.1 mg/ml histone H1, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.58 Example 58

### CaMKIV (h) Assay

In a final reaction volume of 25 μl, CaMKIV (h) (5-10 mU) is incubated with 40 mM Hepes pH 7.4, 5 mM CaCl₂, 30 μg/ml calmodulin, 30 μM KKLNRTLSVA (SEQ ID NO:11), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.59 Example 59

### Blk (m) Assay

In a final reaction volume of 25 μl, Blk (m) (5-10 mU) is incubated with 50 mM Tris pH 7.5, 0.1 mM EGTA, 0.1 mM NaVanadate, 0.1% β-mercaptoethanol, 0.1 mg/ml poly(Glu, Tyr) 4:1, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

### 6.60 Example 60

### Syk (h) Assay

In a final reaction volume of 25 μl, Syk (h) (5-10 mU) is incubated with 50 mM Tris pH 7.5, 0.1 mM EGTA, 0.1 mM NaVanadate, 0.1% β-mercaptoethanol, 0.1 mg/ml poly(Glu, Tyr) 4:1, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

### 6.61 Example 61

### CSK (h) Assay

In a final reaction volume of 25 μl, CSK (h) (5-10 mU) is incubated with 50 mM Tris pH 7.5, 0.1 mM EGTA, 0.1 mM NaVanadate, 0.1% β-mercaptoethanol, 0.1 mg/ml poly(Glu, Tyr) 4:1, 10 mM MnCl₂, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

### 6.62 Example 62

### Lyn (m) Assay

In a final reaction volume of 25 μl, Lyn (m) (5-10 mU) is incubated with 50 mM Tris pH 7.5, 0.1 mM EGTA, 0.1 mM NaVanadate, 0.1% β-mercaptoethanol, 0.1 mg/ml poly(Glu, Tyr) 4:1, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

### 6.63 Example 63

### CDK3/cyclinE (h) Assay

In a final reaction volume of 25 μl, CDK3/cyclinE (h) (5-10 mU) is incubated with 8 mM MOPS pH 7.0, 0.2 mM EDTA, 0.1 mg/ml histone H1, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.64 Example 64

### CDK5/p35 (h) Assay

In a final reaction volume of 25 μl, CDK5/p35 (h) (5-10 mU) is incubated with 8 mM MOPS pH 7.0, 0.2 mM EDTA, 0.1 mg/ml histone H1, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.65 Example 65

### CDK2/cyclinE (h) Assay

In a final reaction volume of 25 μl, CDK2/cyclinE (h) (5-10 mU) is incubated with 8 mM MOPS pH 7.0, 0.2 mM EDTA, 0.1 mg/ml histone H1, 10 mM MgAcetate and [γ-33p-ATP] (specific activity approx. 500 cpm/pmol, concentration as required). The reaction is initiated by the addition of Mg²⁺ [γ-33P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.66 Example 66

### CDK6/cyclinD3 (h) Assay

In a final reaction volume of 25 μl, CDK6/cyclinD3 (h) (5-10 mU) is incubated with 8 mM MOPS pH 7.0, 0.2 mM EDTA, 0.1 mg/ml histone H1, 10 mM MgAcetate and [γ-33p-ATP] (specific activity approx. 500 cpm/pmol, concentration as required). The reaction is initiated by the addition of Mg²⁺ [γ-33P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.67 Example 67

### CDK7/cyclinH/MAT1 (h) Assay

In a final reaction volume of 25 μl, CDK7/cyclinH/MAT1 (h) (5-10 mU) is incubated with 8 mM MOPS pH 7.0, 0.2 mM EDTA, 500 μM peptide, 10 mM MgAcetate and [γ-³³P-ATP] [specific activity approx. 500 cpm/pmol, concentration as required). The reaction is initiated by the addition of Mg²⁺ [γ-33 P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.68 Example 68

### Rsk3 (h) Assay

In a final reaction volume of 25 μl, Rsk3 (h) (5-10 mU) is incubated with 8 mM MOPS pH 7.0, 0.2 mM EDTA, 30 μM KKKNRTLSVA (SEQ ID NO:11), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.69 Example 69

### IR (h) Assay

In a final reaction volume of 25 μl, IR (h) (5-10 mU) is incubated with 50 mM Tris pH 7.5, 0.1 mM EGTA, 0.1 mM NaVanadate, 0.1% β-mercaptoethanol, 250 μM KKSRGDYMTMQIG (SEQ ID NO: 15), 10 mM MnCl₂, 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.70 Example 70

### IGF-1R (h) Assay

In a final reaction volume of 25 μl, IGF-IR (h) (5-10 mU) is incubated with 50 mM Tris pH 7.5, 0.1 mM EGTA, 0.1 mM NaVanadate, 0.1% β-mercaptoethanol, 250 μM KKKSPGEYVNIEFG (SEQ ID NO:16), 10 mM MnCl₂, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.71 Example 71

### PKBP (h) Assay

In a final reaction volume of 25 μl, PKBP (h) (5-10 mU) is incubated with 8 mM MOPS pH 7.0, 0.2 mM EDTA, 30 μM GRPRTSSFAEGKK (SEQ ID NO:2), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.72 Example 72

### FGFR3 (h) Assay

In a final reaction volume of 25 μl, FGFR3 (h) (5-10 mU) is incubated with 50 mM Tris pH 7.5, 0.1 mM EGTA, 0.1 mM NaVanadate, 0.1% β-mercaptoethanol, 0.1 mg/ml poly(Glu, Tyr) 4:1, 10 mM MnCl₂, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

### 6.73 Example 73

### PDGFRα(h) Assay

In a final reaction volume of 25 μl, PDGFRα (h) (5-10 mU) is incubated with 8 mM MOPS pH 7.0, 0.2 mM EDTA, 0.1 mg/ml poly(Glu, Tyr) 4:1, 10 mM MnCl₂, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

### 6.74 Example 74

### PDGFRβ(H) Assay

In a final reaction volume of 25 μl, PDGFRβ (h) (5-10 mU) is incubated with 8 mM MOPS pH 7.0, 0.2 mM EDTA, 0.1 mg/ml poly(Glu, Tyr) 4:1, 10 mM MnCl₂, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

### 6.75 Example 75

### MAPK2(h) Assay

In a final reaction volume of 25 μl, MAPK2(h) (5-10 mU) is incubated with 25 mM Tris pH 7.5, 0.02 mM EGTA, 0.33 mg/ml myelin basic protein, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.76 Example 76

### ROCK-II(h) Assay

In a final reaction volume of 25 μl, ROCK-II (h) (5-10 mU) is incubated with 50 mM Tris pH 7.5, 0.1 mM EGTA, 30 μM KEAKEKRQEQIAKRRRLSSLRASTSKSGGSQK (SEQ ID NO:8), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.77 Example 77

### PKA(h) Assay

In a final reaction volume of 25 μl, PKA(h) (5-10 mU) is incubated with 8 mM MOPS pH 7.0, 0.2 mM EDTA, 30 μM LRRASLG (Kemptide; SEQ ID NO:10), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 [Il of a 3% phosphoric acid solution. 10 μl of the reaction is then spotted onto a P30 filtermat and washed three times for 5 minutes in 50 mM phosphoric acid and once in methanol prior to drying and scintillation counting.

### 6.78 Example 78

### Rsk1(r) Assay

In a final reaction volume of 25 μl, Rsk1(r) (5-10 mU) is incubated with 8 mM MOPS pH 7.0, 0.2 mM EDTA, 30 μM KKKNRTLSVA (SEQ ID NO:11), 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.79 Example 79

### Rsk2(h) Assay

In a final reaction volume of 25 μl, Rsk2(h) (5-10 mU) is incubated with 8 mM MOPS pH 7.0, 0.2 mM EDTA, 30 μM KKKNRTLSVA (SEQ ID NO:11), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.80 Example 80

### PAK2(h) Assay

In a final reaction volume of 25 μl, PAK2(h) (5-10 mU) is incubated with 8 mM MOPS pH 7.0, 0.2 mM EDTA, 30 μM KEAKEKRQEQIAKRRRLSSLRASTSKSGGSQK (SEQ ID NO:8), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.81 Example 81

### Fes(h) Assay

In a final reaction volume of 25 μl, Fes(h) (5-10 mU) is incubated with 8 mM MOPS pH 7.0, 0.2 mM EDTA, 0.1 mg/ml poly(Glu, Tyr) 4:1, 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

### 6.82 Example 82

### Yes(h) Assay

In a final reaction volume of 25 μl, Yes(h) (5-10 mU) is incubated with 8 mM MOPS pH 7.0, 0.2 mM EDTA, 0.1 mg/ml poly(Glu, Tyr) 4:1, 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

### 6.83 Example 83

### ABL(m) Assay

In a final reaction volume of 25 μl, ABL(m) (5-10 mU) is incubated with 8 mM MOPS pH 7.0, 0.2 mM EDTA, 50 μM EAIYAAPFAKKK (SEQ ID NO:17), 10 mM MgAcetate and [γ-³³P-ATP] (specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.84 Example 84

### PKCε(h) Assay

In a final reaction volume of 25 μl, PKCε(h) (5-10 mU) is incubated with 20 mM Hepes pH 7.4, 0.03% Triton X-100, 0.1 mg/ml phosphatidylserine, 10 μg/ml diacylglycerol, 50 μM ERMRPRKRQGSVRRRV (SEQ ID NO: 18), 10 mM MgAcetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### 6.85 Example 85

### Synthesis of 3-(6-Methoxynaphthalen-2-yl)-5-(5-Morpholin-4-Ylmethyl-1H-[1,2,4]Triazol-3-yl)-1H-Indazole (CC001)

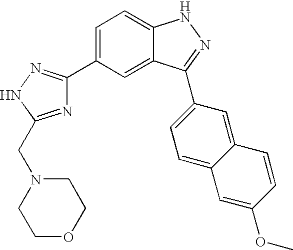

A. Morpholin-4-yl-acetic acid hydrazide

A solution of methyl morpholinoacetate (2.43 g, 15.3 mmol), ethanol (20 mL) and hydrazine (0.53 mL, 16.8 mmol) was stirred at 90° C. for 18 hours. The mixture was concentrated and dried to provide the title compound (2.30 g, 95%): ES-MS (m/z) 160 [M+1]⁺.

B. 3-(6-methoxynaphthalen-2-yl)-5-(5-morpholin-4-ylmethyl-1H-[1,2,4]triazol-3-yl)-1H-indazole

The title compound was prepared as follows: To a flask was charged 3-(6-methoxynaphthalen-2-yl)-1H-indazole-5-carboximidic acid ethyl ester (1.0 g, 2.62 mmol) and morpholin-4-yl-acetic acid hydrazide (1.67 g, 10.5mmol). After 10 minutes, the hydrazide prepared as described in Example 422 A of International Publication No. WO 02/10137 (1.05 g, 7.31 mmol):was added and the mixture was heated at 90° C. for 18 hours. The mixture was concentrated and purified by preparatory HPLC to provide the title compound (481 mg, 42%): ¹H NMR (CD₃OD) δ 8.82 (s, 1H) 8.42 (s, 1H) 8.08 (d, 2H) 7.93 (t, 2H) 7.65 (br s, 1H) 7.30 (s, 1H) 7.21 (d, 1H) 4.15 (s, 3H) 3.72 (m, 6H) 2.59 (br s, 4H); ES-MS (m/z) 441 [M+1]⁺.

### 6.86 Example 86

### Synthesis of 5-(5-Isobutyl-1H-[1,2,4]Triazol-3-yl)-3-[6-(2-Pyrrolidin-1-yl-Ethoxy)-Naphthalen-2-yl]-1H-Indazole (CC002)

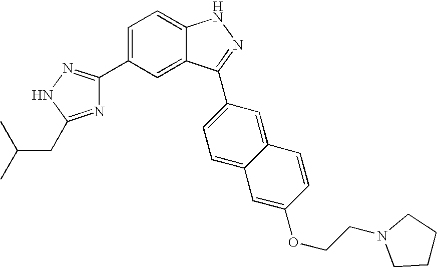

A. 4-Fluoro-3-[hydroxy-(6-methoxy-naphthalen-2-yl)-methyl]-benzonitrile

To a cooled solution (−78° C.) of LDA (24.5 mL, 49 mmol) in THF (40 mL) was added 4-fluorobenzonitrile (5.45 g, 45 mmol) in THF (30 mL) and 6-methoxy-2-naphthaldehyde (9.13 g, 49 mmol) in THF (40 mL). The reaction was stirred at -78° C. for 1 hour and then allowed to warm to room temperature over a period of 2 hours. The reaction was quenched with ice and THF was removed in vacuo. The aqueous solution was then extracted with ethyl acetate. The organic phase was dried over magnesium sulfate, filtered and the solvent was removed in vacuo. The crude material was purified by column chromatography (SiO₂, 4:1 hexanes: ethyl acetate) to provide the title compound (5.5 g, 40% yield). ES-MS (m/z) 308 [M+1]⁺.

B. 4-Fluoro-3-(6-methoxy-naphthalene-2-carbonyl)-benzonitrile

In a round bottom flask, pyridinium chlorochromate (6.4 g, 29.7 mmol) was taken up in a slurry of dichloromethane (40 mL). 4-Fluoro-3-[hydroxy-(6-methoxy-naphthalen-2-yl)-methyl]-benzonitrile (6.08 g, 19.8 mmol) in dichloromethane (40 mL) was added. The reaction turned black. The mixture was stirred at room temperature for 5 hours, after which it was filtered through a pad of Celite and the solvent removed in vacuo. The crude material was purified by column chromatography (SiO₂, 4:1 hexanes: ethyl acetate—1:1 hexanes: ethyl acetate) to provide the title compound (4.54 g, 75% yield). ES-MS (m/z) 306 [M+1]⁺.

C. 4-Fluoro-3-(6-hydroxy-naphthalene-2-carbonyl)-benzonitrile

4-Fluoro-3-(6-methoxy-naphthalene-2-carbonyl)-benzonitrile (3.976 g, 13 mmol) was dissolved in dichloromethane (350 mL) and cooled under nitrogen in an ice bath. Boron tribromide (12.28 mL, 130 mmol) was slowly added and the reaction was allowed to stir overnight at room temperature. The reaction was quenched with ice, neutralized with sodium bicarbonate and extracted with dichloromethane. The organic layer was dried over magnesium sulfate, filtered and the solvent was removed in vacuo. The crude material was purified by column chromatography (SiO₂, 7:3 hexanes: ethyl acetate) to provide the title compound (2.5 g, 66% yield). ES-MS (m/z) 292 [M+1]⁺.

D. 4-Fluoro-3-[6-(2-pyrrolidin-1-yl-ethoxy)-naphthalene-2-carbonyl]-benzonitrile

4-Fluoro-3-(6-hydroxy-naphthalene-2-carbonyl)-benzonitrile (2.7 g, 9.3 mmol) was dissolved in dioxane (22 mL). Tetraethylammonium bromide (195 mg, 0.93 mmol) was added, followed by sodium hydroxide (1.12 g in 1.6 mL of water). 1-(2-chloroethyl) pyrrolidine hydrochloride (1.74 g, 10.23 mmol) was added and the reaction mixture was stirred at 55° C. for 4 hrs. Solvent was removed in vacuo and water was added. Reaction mixture was neutralized to pH=7 and thoroughly extracted with ethyl acetate. The organic layer was dried over magnesium sulfate, filtered and solvent removed in vacuo to provide the title compound (3.16 g, 87% yield). ES-MS (m/z) 389 [M+1]⁺.

E. 3-[6-(2-pyrrolidin-1-yl-ethoxy)-naphthalen-2-yl] -1H-indazole-5-carbonitrile

To a solution of 4-fluoro-3-[6-(2-pyrrolidin-1-yl-ethoxy)-naphthalene-2-carbonyl]-benzonitrile (3.16 g, 8.14 mmol) in toluene (40 mL) hydrazine monohydrate (0.87 mL, 17.9 mmol) was added and the reaction mixture was heated at 65° C. overnight. The solvent was removed in vacuo and the crude material was purified by column chromatography (SiO₂, 1% triethylamine/ethyl acetate) to provide the title compound (2.0 g, 65% yield). ES-MS (m/z) 383 [M+1]⁺.

F. 3-[6-(2-Pyrrolidin-1-yl-ethoxy)-naphthalen-2-yl] -1H-indazole-5-carboximidic acid ethyl ester, hydrochloride

The title compound was prepared as follows: A solution of 3-[6-(2-pyrrolidin-1-yl-ethoxy)-naphthalen-2-yl]-1H-indazole-5-carbonitrile (2 g, 5.23 mmol) in 400 mL ethanol was cooled to 0° C. HCl gas was bubbled through the reaction mixture for 30 minutes. The reaction vessel was sealed and the mixture stirred at room temperature for 20 h. The reaction mixture was diluted with diethyl ether and the precipitate was filtered and washed with diethyl ether. The white solid was dried in a 40 ° C. vacuum overnight to provide the title compound. 2 g of a yellow solid were obtained (89% yield). ES-MS (m/z) 429 [M+1]⁺.

G. 5-(5-isobutyl-1H-[1,2,4]triazol-3-yl)-3-[6-(2-pyrrolidin-1-yl-ethoxy)-naphthalen-2-yl]-1H-indazole

The title compound was prepared as follows: To a solution of 3-[6-(2-pyrrolidin-1-yl-ethoxy)-naphthalen-2-yl]-1H-indazole-5-carboximidic acid ethyl ester (2 g, 4.67 mmol) was added 3-methyl-butyric acid hydrazide (1.63 g, 14.04 mmol), triethylamine (13.04 mL, 93.44 mmol) in methanol (20 mL). The reaction was stirred at 100° C. in a sealed pressure vessel for 4h. The reaction yielded 378.5 mg (17% yield) of the title compound after concentration and purification by HPLC (20-65% water/acetonitrile). ¹H NMR (methanol-d₄, 300 MHz) δ 8.82 (s, 1H), 8.47 (s, 1H), 8.10 (dd, 2H), 7.97 (dd, 2H), 7.69 (d, 1H), 7.39 (s, 1H), 7.29 (d, 1H), 4.47 (t, 2H), 3.72 (m, 4H), 3.28 (m, 2H), 2.75 (d, 2H), 2.07 (m, 5H), 1.01 (d, 6H). ES-MS (m/z) 481 [M+1]⁺.

### 6.87 Example 87

### Synthesis of 5-[5-(2,2-Dimethyl-propyl)-1H-[1,2,4]triazol-3-yl]-3-[6-(pyridine-2-ylmethoxy)-naphthalen-2-yl]-1H-indazole (CC004)

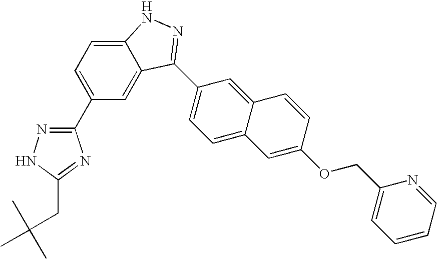

A. 2-(6-Bromo-naphthalen-2-yloxymethyl)-pyridine

To an ice bath cooled solution of 6-bromonaphthol (6.13 g, 27.5mmol), pyridine-2-yl-methanol (2.64 mL, 27.5 mmol, 1.0 eq.), and triphenylphosphine (10.8 g, 41.30 mmol, 1.5 eq.) in TFIF was added diisopropyl azodicarboxylate (8.12 mL, 41.3 mmol, 1.5eq.). The reaction was monitored by TLC (30% ethyl acetate/hexanes) and was complete after 24 hours. Solvent was removed in vacuo and subjected to Biotage column chromatography to afford 9.00 g (100% yield) of the title compound as tan solids. ES-MS (m/z) 313 [M+1].

B. 3-[6-(Pyridin-2-ylmethoxy)-naphthalen-2-yl]-1-(tetrahydro-pyran-2-yl)-1H-indazole-5-carbonitrile

To a solution of 2-(6-Bromo-naphthalen-2-yloxymethyl)-pyridine (4.97 g, 15.8 mmol) in DMF (50 mL) was added bis(pinnacalato)diboron (4.02 g, 15.8 mmol, 1.0 eq.) and potassium acetate (4.66 g, 47.6 mmol, 3.0 eq.) and palladium II chloride (bis-diphenyl phosphino ferrocene) dichloromethane (1.29 g, 10% mmol). Reaction was heated at 80° C. overnight and LCMS confirmed formation of boronate ester complex. To the reaction was added 3-bromo-1-perhydro-2H-pyran-2-yl-1H-indazole-5-carbonitrile (4.85 g, 15.8 mmol, 1.0 eq.) and potassium phosphate (10.09 g, 47.6 mmol, 3.0 eq.) and was stirred at 80° C. overnight. Reaction was monitored by LCMS and was complete after 24 hours. The solvent was removed in vacuo and the residue was washed with water, extracted with ethyl acetate, and subjected to Biotage column chromatography (60% ethyl acetate/hexanes) to afford 2.10 g (30% yield) of the title compound as tan solids. ES-MS (m/z) 460 [M+1].

C. 3-[6-(Pyridin-2-ylmethoxy)-naphthalen-2-yl]-1H-indazole-5-carboximidic acid ethyl ester

To a dry ice/acetone bath cooled solution of 3-[6-(Pyridin-2-ylmethoxy)-naphthalen-2-yl]-1-(tetrahydro-pyran-2-yl)-1H-indazole-5-carbonitrile (2.00 g, 4.30 mmol) in ethanol (500 mL) was bubbled through HCl(g) for twenty minutes. Reaction was monitored by LCMS and was complete after 72 hours. Solvent was removed in vacuo and was triturated with diethyl ether. The solids were filtered to afford 1.05 g (58% yield) of the title compound as off white solids. ES-MS (m/z) 422 [M+1].

D. 5-[5-(2,2-Dimethyl-propyl)-1H-[1,2,4]triazol-3-yl]-3-[6-(pyridine-2-ylmethoxy- naphthalen-2-yl]-1H-indazole

To a solution of 3-[6-(Pyridin-2-ylmethoxy)-naphthalen-2-yl]-1H-indazole-5-carboximidic acid ethyl ester (0.50 g, 1.18 mmol) was added Dimethyl-butyric acid hydrazide (0.62 g, 4.72 mmol, 4.0 eq.) and triethylamine (3.28 mL, 23.6 mmol, 20.0 eq.). The reaction was heated to 90 degrees in a sealed tube overnight. The reaction was monitored by LCMS and was complete after 24 hours. The solvent was removed in vacuo and was subjected to Prep HPLC (20-80% acetonitrile/water+0.1% TFA) to afford 60 mgs (9% yield) of the title compound as white solids. ¹H NMR (CD3OD) δ 8.98(s, 1H), 8.70(d, 1H), 8.60(s, 1H), 8.35(dd, 2H), 8.10(d, 1H), 8.00(dd, 2H), 7.80(m, 2H), 7.55-7.40(m, 3H), 5.45(s, 2H), 2.95(s, 2H), 1.05(s, 9H). ES-MS (m/z) 566 [M+1].

### 6.88 Example 88

### Synthesis of 2-[((2s)-1-ethylpyrrolidin-2-yl)methoxy]-6-{5-[3-(2,2-Di methylpropyl)(1H-1,2,4,-triazol-5-yl)](1H-indazol-3-yl)}naphthalene (CC005)

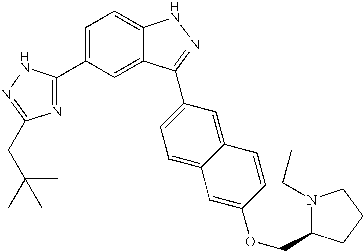

A. 2-[((2S)-1-ethylpyrrolidin-2-yl)methoxy]-6-bromonapthalene

The title compound was prepared as follows: To a solution of 6-bromo-2-napthol (6.95 g., 31.2 mmol), ((2S)-1-ethylpyrrolidin-2-yl)methan-1-ol (6.20 g., 48.13 mmol), and triphenylphosphine (12.26 g., 46.8 mmol) in THF was added diisobutylazodicarboxylate (9.23 mL, 46.8 mmol). The solution stirred for twenty minutes at ambient temperature and monitored via TLC until completion of the reaction. The solvent was evaporated under reduced pressure to give an oil. A mixture of diethyl ether: hexanes (1:1) was added to the oil and sonicated for 5 minutes to precipitate out triphenylphosphine oxide. The white solid was filtered through celite and the resultant filtrate condensed under reduced pressure to an oil. The oil was purified via silica gel chromatography (3-10% methanol/dichloromethane) to afford the title compound (11.0 g., >100% yield). ES-MS (m/z) 334[M+1]+, 336 [M+2]+.

B. 3-{6-[((2S)-1-ethylpyrrolidin-2-yl)methoxy](2-naphthyl)}-1-perhydro-2H-pyran-2-yl-1H-indazole-5-carbonitrile

The title compound was prepared as follows: A mixture of 2-[((2S)-1-ethylpyrrolidin-2-yl)methoxy]-6-bromonaphthalene (4.46 g, 13.39 mmol), [1,1′-bis(diphenylphosphinoferrocene) complex with dichloromethane (1:1) (1.13 g., 1.385 mmol), potassium acetate (4.07 g., 41.55 mmol), and bis(pinnacolato)-diboron (3.51 g., 13.85 g) in dimethylformamide (70 mL) was heated to 95° C. for three hours. The reaction was monitored by TLC and ES-MS to assure boronate ester formation. 3-Bromo-1-(tetrahydro-pyran-2-yl)-1H-indazole-5-carbonitrile (4.23 g., 13.85 mmol) and potassium carbonate (10.85 g., 41.55 mmol) were then added and heated for an additional 16 hours at 95° C. The resultant mixture was then condensed under reduced pressure to afford a black oil. The oil was then diluted with ethyl acetate and filtered through celite and solvent removed under reduced pressure. The resultant oil was purified via silica gel chromatography (10-15% methanol/dichloromethane) to afford the title compound (1.05 g., 16% yield). ES-MS (m/z) 481[M+1]+.

C. (3-{6-[((2S)-1-ethylpyrrolidin-2-yl)methoxy](2-naphthyl}(1H-indazol-5-bis(diphenylphosphinoferrocene)

The title compound was prepared as described as follows: 3-{6-[((2S)-1-ethylpyrrolidin-2-yl)methoxy](2-naphthyl)}-1-perhydro-2H-pyran-2-yl-1H-indazole-5-carbonitrile (4.6 g., 8.85 mmol) was dissolved in ethanol (800 mL) and cooled to 0° C. Hydrogen chloride gas was then bubbled into solution for twenty minutes. The acidified mixture was then stirred at room temperature for 16 hours. The resultant solution was condensed under reduced pressure to afford a solid. The solid was washed with diethyl ether and filtered through a buchner funnel and dried under vacuum to afford the title compound (1.10 g., 98% yield). ES-MS (m/z) 443[M+1]+.

D. 2-[((2S)-1-ethylpyrrolidin-2-yl)methoxy]-6-{5-[3-(2,2-dimethylpropyl) triazol-5-yl)](1H-indazol-3-yl)}naphthalene

The title compound was prepared as described as follows: To a solution of (3-{6-[((2S)-1-ethylpyrrolidin-2-yl)methoxy](2-naphthyl}(1H-indazol-5-yl))ethoxymethanimine (0.550 g., 1.24 mmol) and N-amino-3,3-dimethylbutanamide (0.323 g., 2.488 mmol) was added cyclopentyl methyl hydrazide (1.4 g., 9.89 mmol) and triethylamine (4.98 g., 49.4 mmol). The mixture was heated to 95° C. in a sealed tube for 16 hours. The solvent was then removed under reduced pressure and the oil purified via preparative HPLC (20-80% acetonitrile/water, 60 mL/min.) to provide the title compound in 100% purity by analytical HPLC (0.061 g., 9.6% yield). 1H-NMR (CHCl3) δ 8.83 (s, 1H), 8.39 (s, 1H), 8.20 (d, 1H), 8.09 (d, 1H), 7.83 (m, 2H), 7.58 (d, 1H), 7.19 (m, 2H), 4.16 (m, 1H), 4.0 (m, 1H), 3.26 (t, 1H), 3.05 (m, 1H), 2.97 (m, 1H), 2.78 (s, 2H), 2.50 (m, 1H), 2.33 (m, 1H), 2.21 (m, 1H), 2.05 (m, 1H), 1.85 (m, 4H), 1.10 (t, 3H), 1.04 (q, 2H). ES-MS (m/z) 509[M+1]⁺.

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

1. A method of treating an individual comprising administering to said individual a single agent, wherein said single agent modulates the activity of at least two kinases, and wherein each of said kinases is selected independently from the group consisting of a CDK kinase, an Rsk kinase, a MAPK kinase, a checkpoint kinase, a Src kinase, Fes, Lyn, Syk.

2. The method of claim 1, wherein said at least two kinases are at least two of cSRC, Yes, Fyn, Lck, Fes, Lyn, Syk, Rsk1, CDK1, CDK2, CDK3, CDK5, CDK6, CDK7, CHK1, CHK2, JNK1, MAPK1, MAPK2, MAPKAP-K5, MEK1, ROCKII, PRK2, PRAK, p70S6 or Aurora-A.

3. The method of claim 2, wherein said at least two kinases are human CDK1, CDK2, cSRC, Yes, MEK1 and Rsk1.

4. The method of claim 1 or claim 2 in which said individual suffers from cancer, a proliferative disorder, an inflammatory disorder, or obesity.

5. The method of claim 1 wherein said single agent is a compound having the structure 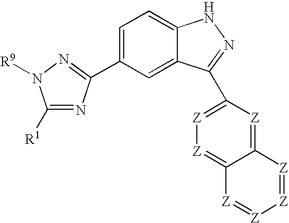
and isomers, prodrugs and pharmaceutically acceptable salts, solvates and hydrates thereof, wherein,
R¹ is —H, -halo, —C₁-C₆ alkyl, —C₁-C₆ alkenyl, —C₁-C₆ alkynyl, —OR³, —N(R⁴)₂, —CN, —NO₂, —C(O)R⁵, —OC(O)R⁵, —NHC(O)R⁵, —SO₂R⁶, -aryl, -heterocycle, -heteroaryl, -cycloalkyl, —(C₁-C₆ alkylene)-R² or —O—(C₁-C₆ alkylene)-R²;
R² is —H, -halo, —C₁-C₆ alkyl, —C₁-C₆ alkenyl, —C₁-C₆ alkynyl, —OR³, —N(R⁴)₂, —CN, —NO₂, —C(O)R⁵, —OC(O)R⁵, —NHC(O)R⁵, —SO₂R⁶, -aryl, -heterocycle, -heteroaryl, or -cycloalkyl;
R³ is independently —H, —C₁-C₆ alkyl, —C₁-C₆ alkenyl, —C₁-C₆ alkynyl, —C₁-C₆ haloalkyl, -cycloalkyl, -aryl, or -heterocycle;
each occurrence of R⁴ is independently —H, —C₁-C₆ alkyl, —C₁-C₆ alkenyl, —C₁-C₆ alkynyl, -cycloalkyl, -aryl, -heterocycle, or —(C₁-C₆ alkylene)-OR³;
R⁵ is —H, —C₁-C₆ alkyl, —C₁-C₆ alkenyl, —C₁-C₆ alkynyl, -cycloalkyl, -aryl, -heterocycle, —OR³, —N(R⁴)₂,
R⁶ is —H, —C₁-C₆ alkyl, —C₁-C₆ alkenyl, —C₁-C₆ alkynyl, —N(R⁴)₂, -cycloalkyl, -aryl, or -heterocycle;
each occurrence of Z is —C(R⁷)— or —N—, wherein up to 3 occurrences of Z can be —N—;
R⁷ is —H, -halo, —C₁-C₆ alkyl, —C₁-C₆ haloalkyl, —O—(C₁-C₆haloalkyl), —C₁-C₆ alkenyl, —C₁-C₆ alkynyl, —OR³, —N(R⁴)₂, —CN, —NO₂, —C(O)R⁵, —OC(O)R⁵, —NHC(O)R⁵, —SO₂R⁶, -aryl, -heterocycle, -cycloalkyl, —C(O)NH—(C₁-C₆ alkylene )ₙ-cycloalkyl, —C(O)NH—(C₁-C₆ alkylene )ₙ-aryl, —C(O)NH—(C₁-C₆ alkylene )ₙ-heterocycle, —(C₁-C₆ alkylene)-cycloalkyl, —(C₁-C₆ alkylene)-aryl, —(C₁-C₆ alkylene)-heterocycle, —O—(C₁-C₆ alkylene)-C(O)R⁵, —O—(C₁-C₆ alkylene)-N(R⁴)₂, —(C₁-C₆ alkylene)-cycloalkyl, —O—(C₁-C₆ alkylene)-aryl, or —O—(C₁-C₆ alkylene)-heterocycle, wherein R⁷ is attached to the bicyclic ring system via a carbon atom and n is 0 or 1; and
R⁹ is —H, —C₁-C₆ alkyl, or cycloalkyl.

6. The method of claim 1 wherein said single agent is a compound having the structure 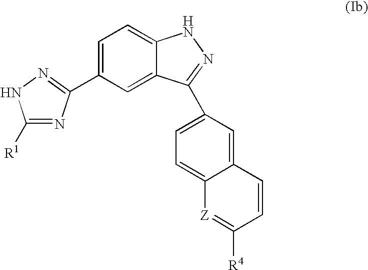
and isomers, prodrugs and pharmaceutically acceptable salts, solvates and hydrates thereof, wherein
R¹ is —H, —C₁-C₆ alkyl, —(C₁-C₆ alkylene)-R² or —O—(C₁-C₆ alkylene)-R²;
R² is —C₁-C₆ alkyl, —C₁-C₆ alkoxy, —OH, —N(R³)₂, -aryl, -heteroaryl, -heterocycle, or -cycloalkyl;
each occurrence of R³ is independently —H, —C₁-C₆ alkyl, or —C₁-C₆ alkylene-(C₁-C₆ alkoxy);
R⁴ is —N(R⁵)₂, —O—C₁-C₆ alkyl, —C(O)NH—(C₁-C₆ alkylene)ₘ-heterocycle, —C(O)-heterocycle, —C(O)NH—(C₁-C₆ alkylene)ₘ-heteroaryl, —C(O)-heteroaryl, —(C₁-C₆ alkylene)-cycloalkyl, —O—(C₁-C₆ alkylene)-N(R⁵)₂, —O—(C₁-C₆ alkylene)ₘ-heterocycle, —O—(C₁-C₆ alkylene)ₘ-heteroaryl, —O—(C₁-C₆ alkylene)ₘ-cycloalkyl or —O—(C₁-C₆ alkylene)ₘ-C(O)R⁵;
Z is —CH— or —N—;
each occurrence of R⁵ is independently —H or —C₁-C₆ alkyl; and
m is 0 or 1.

7. The method of claim 1, wherein said single agent is 3-(6-methoxynaphthalen-2-yl)-5-(5-morpholin-4-ylmethyl-1H-[1,2,4]triazol-3-yl)-1H-indazole; 5-(5-isobutyl-1H-[1,2,4]triazol-3-yl)-3-[6-(2-pyrrolidin-1-yl-ethoxy)-naphthalen-2-yl]-1H-indazole; 5-[5-(2,2-dimethyl-propyl)-1H-[1,2,4]triazol-3-yl]-3-[6-(pyridine-2-ylmethoxy)-naphthalen-2-yl]-1H-indazole; or 2-[((2s)-1-ethylpyrrolidin-2-yl)methoxy]-6-{5-[3-(2,2-Di methylpropyl)(1H-1,2,4,-triazol-5-yl)](1H-indazol-3-yl)}naphthalene.

8. The method of claim 1 wherein said single agent is a non-indazole small molecule, a peptide or a polynucleotide.

9. The method of claim 1, wherein said single agent is an indazole-containing compound other than 5-(5-cyclopentyl-1H-[1,2,4]triazol-3-yl)-3-(4-fluoro-phenyl)-1H-indazole or 3-(5-(1H-1,2,4-triazol-3-yl)(1H-indazol-3-yl))phenyl-N-cyclopentylcarboxamide.

10. The method of claim 1, wherein said single agent inhibits activities of each of at least 7 kinases shown in Table 2 by at least 90%, relative to activities of said at least 7 kinases in the absence of said single agent, wherein said single agent is present at a concentration of 3 μM.

11. The method of claim 1, wherein said single agent inhibits activities of each of human CDK1, CHK2, PRK2 and ROCK-II by 90% or more, relative to activities of said CDK1, CHK2, PRK2 and ROCK-II under equivalent conditions in the absence of said single agent, wherein said single agent is present at a concentration of 3 μM.

12. The method of claim 1, wherein said single agent is present in a pharmaceutical composition.

13. The method of claim 1, wherein said intividual is treated with a plurality of single agents.

14. The method of claim 4, wherein said individual is additionally administered a non-single agent cancer drug or treatment.

15. The method of claim 4, wherein said individual is treated for lung cancer or colon cancer.


SEQUENCE LISTING (18 sequences)

SEQ ID NO: 1 - protein, 15 aa, Artificial Sequence
  Synthetic substrate peptide Syntide-2
PLARTLSVAGLPGKK

SEQ ID NO: 2 - protein, 13 aa, Artificial Sequence
  Synthetic peptide
GRPRTSSFAEGKK

SEQ ID NO: 3 - protein, 26 aa, Artificial Sequence
  Phospho GS2 peptide
YRRAAVPPSPSLSRHSSPHQSEDEEE

SEQ ID NO: 4 - protein, 15 aa, Artificial Sequence
  Synthetic peptide
AMARAASAAALARRR

SEQ ID NO: 5 - protein, 23 aa, Artificial Sequence
  Synthetic peptide
KKKVSRSGLYRSPSMPENLNRPR

SEQ ID NO: 6 - protein, 10 aa, Artificial Sequence
  Synthetic peptide
RRRDDDSDDD

SEQ ID NO: 7 - protein, 15 aa, Artificial Sequence
  Cdc2 peptide
KVEKIGEGTYGVVYK

SEQ ID NO: 8 - protein, 32 aa, Artificial Sequence
  Synthetic peptide
KEAKEKRQEQIAKRRRLSSLRASTSKSGGSQK

SEQ ID NO: 9 - protein, 9 aa, Artificial Sequence
  Synthetic peptide
KKRNRTLTV

SEQ ID NO: 10 - protein, 7 aa, Artificial Sequence
  Kemptide
LRRASLG

SEQ ID NO: 11 - protein, 10 aa, Artificial Sequence
  Synthetic peptide
KKLNRTLSVA

SEQ ID NO: 12 - protein, 11 aa, Artificial Sequence
  Synthetic peptide
AKRRRLSSLRA

SEQ ID NO: 13 - protein, 39 aa, Artificial Sequence
  PDKtide
KTFCGTPEYLAPEVRREPRILSEEEQEMFRDFDYIADWC

SEQ ID NO: 14 - protein, 14 aa, Artificial Sequence
  Synthetic peptide
KRRRALSVASLPGL

SEQ ID NO: 15 - protein, 13 aa, Artificial Sequence
  Synthetic peptide
KKSRGDYMTMQIG

SEQ ID NO: 16 - protein, 14 aa, Artificial Sequence
  Synthetic peptide
KKKSPGEYVNIEFG

SEQ ID NO: 17 - protein, 12 aa, Artificial Sequence
  Synthetic peptide
EAIYAAPFAKKK

SEQ ID NO: 18 - protein, 16 aa, Artificial Sequence
  Synthetic peptide
ERMRPRKRQGSVRRRV